(19) United States
(12) Patent Application Publication
(10) Pub. No.: US 2010/0081692 A1
(43) Pub. Date: Apr. 1, 2010

(54) Novel 1-Benzyl-4-Piperidinamines that are Useful in the Treatment of COPD and Asthma

(75) Inventors: Martin Hemmerling, Lund (SE); Nafizal Hossain, Lund (SE); Svetlana Ivanova, Lund (SE); Tesfaledet Mussie, Lund (SE); Igor Shamovsky, Lund (SE); Asa Sjoholm Timen, Lund (SE)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/092,311

(22) PCT Filed: Oct. 31, 2006
(86) PCT No.: PCT/SE06/01227

§ 371 Date: May 1, 2008

(30) Foreign Application Priority Data
Nov. 2, 2005 (SE) 0502430-2

(51) Int. Cl. A61K 31/4468; C07D 211/56; C07D 401/12; A61K 31/454; A61P 11/00

(52) U.S. Cl. 514/326; 546/223; 546/210; 514/329

(57) ABSTRACT

The invention provides 1-benzyl-4-piperidinamines of the general formula (I), processes for their preparation, pharmaceutical compositions containing them and their use in therapy. The compounds are useful in the treatment of respiratory diseases such as chronic obstructive pulmonary disease and asthma. The compounds are inhibitors of the CCR1 receptor.

The present invention relates to novel compounds, processes for their preparation, pharmaceutical compositions containing them and their use in therapy.

Chemokines play an important role in immune and inflammatory responses in various diseases and disorders, including asthma, allergic diseases, rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C) and Cys-Cys (C—C) families. These are distinguished on the basis of a single amino acid insertion between the NH-proximal pair of cysteine residues and sequence similarity.

Chemokines are attractants and activators of monocytes, lymphocytes and neutrophils.

The C—C chemokines include potent chemoattractants such as human monocyte chemotactic proteins 1-3 (MCP-1, MCP-2 and MCP-3), RANI ES (Regulated on Activation, Normal T Expressed and Secreted), eotaxin and the macrophage inflammatory proteins 1α and 1β (MIP-1α and MIP-1β). The C—X—C chemokines include several potent chemoattractants such as interleukin-8 (IL-8) and neutrophil-activating peptide 2 (NAP-2).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4.

Chemokine Receptor 1 (CCR1) is highly expressed in tissues affected in different autoimmune, inflammatory, proliferative, hyper proliferative and immunologically mediated diseases, e.g. asthma, chronic obstructive pulmonary disease, multiple sclerosis and rheumatoid arthritis. Therefore, inhibiting CCR1-mediated events is expected to be effective in the treatment of such conditions.

International publication number WO01/98273 describes benzyl piperidines which modulate MIP-1α chemokine receptor activity.

A desirable property for a drug acting at the CCR1 receptor is that it has high potency e.g. as determined by its ability to inhibit the activity of the CCR1 receptor. It is also desirable for such drugs to possess good selectivity and pharmacokinetic properties in order to further enhance drug efficacy. As an example, it can be advantageous for such drugs to exhibit low activity against the human ether-a-go-go-related gene (hERG)-encoded potassium channel.

In accordance with the present invention, there is provided a compound of formula

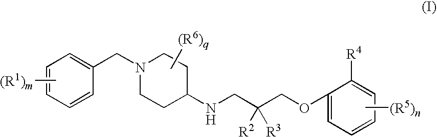

is wherein
m is 0, 1 or 2;
n is 0, 1 or 2;
q is 0, 1, 2, 3 or 4;
each R¹ independently represents halogen, C₁-C₆ alkyl, C₁-C₆ alkoxy, OCF₃ or CF₃;
R² represents a hydrogen atom, C₁-C₆ alkyl or C₃-C₇ cycloalkyl;
R³ represents a hydrogen atom or hydroxyl group;
R⁴ represents —C₁-C₆ alkyl-COOH, —C₁-C₆ alkoxy-COOH, —C₃-C₄ cycloalkyl-COOH, —(CH₂)ₜ-tetrazole;
t is 0, 1 or 2;
each R⁵ independently represents halogen, C₁-C₆ alkyl, OCF₃ or CF₃;
each R⁶ represents C₁-C₆ alkyl;

or a pharmaceutically acceptable salt thereof.

In the context of the present specification, an alkyl substituent group or an alkyl moiety in a substituent group may be linear or branched.

It will be appreciated that throughout the specification, the number and nature of substituents on rings in the compounds of the invention will be selected so as to avoid sterically undesirable combinations.

Each R¹ independently represents halogen (e.g. chlorine, bromine, fluorine, or iodine), C₁-C₆ alkyl, preferably C₁-C₄ alkyl (e.g. methyl), C₁-C₆ alkoxy, preferably C₁-C₄ alkoxy (e.g. methoxy), OCF₃, or CF₃. In an embodiment of the invention, each R¹ independently represents halogen, methyl, methoxy, OCF₃ or CF₃. Preferably, each R¹ represents halogen, particularly chlorine.

m may be 0, 1 or 2. Preferably, m is 1 or 2, particularly 1.

In an embodiment of the present invention, m is 1 and R¹ is a chlorine atom in the 4-position of the benzene ring relative to the carbon atom to which the CH₂ linking group is attached.

R² represents a hydrogen atom, a C₁-C₆ alkyl, preferably C₁-C₄ alkyl (e.g. methyl, ethyl, n-propyl, isopropyl, n-butyl, isobutyl, tert-butyl), or C₃-C₇ cycloalkyl (e.g. cyclopropyl, cyclobutyl, cyclopentyl or cyclohexyl). Preferably, R² represents a hydrogen atom, a methyl group or an ethyl group, especially a methyl group.

R³ represents a hydrogen atom or a hydroxyl group. Preferably, R³ represents a hydroxyl group.

R⁴ represents —C₁-C₆ alkyl, preferably —C₁-C₄ alkyl-COOH. In an embodiment of the present invention, R⁴ represents (CH₂)ₚ—COOH, where p represents 0, 1, 2, 3 or 4. In a preferred aspect of this embodiment, p is 1 or 2.

R⁴ represents —C₁-C₆ alkoxy, preferably —C₁-C₄ alkoxy-COOH. In an embodiment of the present invention, R⁴ represents —C₁-C₂ alkoxy-COOH,

R⁴ represents —C₃-C₄ cycloalkyl-COOH. In an embodiment of the present invention, R⁴ represents -cyclopropyl-COOH.

R⁴ represents —(CH₂)ₜ-tetrazole, wherein t is 0, 1 or 2. In an embodiment of the present invention t is 0 or 1.

In one aspect of this embodiment R⁴ represents CH₂—COOH, (CH₂)₂—COOH, -methoxy-COOH, -ethoxy-COOH, -cyclopropyl-COOH, -tetrazole or -methyl-tetrazole.

Each R⁵ represents halogen (e.g. chlorine, bromine, fluorine, or iodine), C₁-C₆ alkyl, preferably C₁-C₄ alkyl (e.g. methyl), OCF₃, or CF₃. In an embodiment of the invention,

each R⁵ independently represents halogen, methyl, OCF₃ or CF₃. Preferably, each R⁵ represents halogen, particularly chlorine or fluorine.

n may be 0, 1 or 2, preferably 0 or 1.

Each R⁶ represents C₁-C₆ alkyl, preferably C₁-C₄ alkyl (e.g. methyl).

q may be 0, 1, 2, 3 or 4. In a preferred embodiment, q is 0, 1, 2 or 4. On one aspect of this embodiment q is 0. In another aspect q is 1. In yet another aspect q is 2.

In an embodiment of the present invention, the substitution pattern of R⁵ is as shown below:

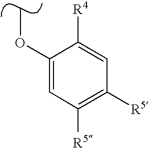

wherein R⁴ is as defined above, and R⁵′ and R⁵″ are each independently hydrogen, halogen, C₁-C₆ alkyl, OCF₃ or CF₃. In a further aspect of this embodiment, R⁵′ and R⁵″ are each independently hydrogen, halogen, methyl, OCF₃ or CF₃. In a further aspect of this embodiment, R⁵′ and R⁵″ are both hydrogen, or one of R⁵′ and R⁵″ represents fluorine or chlorine and the other of R⁵′ and R⁵″ represents hydrogen.

In a further aspect of this embodiment, when R⁴ represents —(CH₂)₂—COOH, R⁵′ and R⁵″ are both hydrogen, or one or both of R⁵′ and R⁵″ are fluorine.

In another aspect of this embodiment, when R⁴ represents —C₁-C₂-alkoxy-COOH, R⁵′ and R⁵— are both hydrogen.

In yet a further aspect of this embodiment, when R⁴ represents —(CH₂)ₜ-tetrazole, t is 0 or 1, R⁵′ and R⁵″ are both hydrogen, or one or both of R⁵ and R⁵″ are fluorine.

In one embodiment, the present invention provides compounds of formula (I) wherein
m is 1 or 2;
n is 0, 1 or 2;
q is 0, 1, 2 or 4;
each R¹ independently represents halogen, C₁-C₄ alkyl or CF₃;
R² represents a hydrogen atom or C₁-C₄ alkyl;
R³ represents a hydrogen atom or hydroxyl group;
R⁴ represents —C₁-C₄ alkyl-COOH, —C₁-C₄ alkoxy-COOH, —C₃-C₄ cycloalkyl-COOH, or —(CH₂)ᵣtetrazole;
t is 0 or 1;
each R⁵ independently represents halogen or CF₃;
each R⁶ represents C₁-C₄ alkyl;

or a pharmaceutically acceptable salt thereof.

In a further embodiment, the present invention provides compounds of formula (I) wherein
m is 1 or 2;
R¹ is chlorine, fluorine, bromine, methyl or CF₃;
n is 0, 1 or 2;
q is 0, 1 or 2;

R² represents a hydrogen atom, a methyl group or ethyl group;
R³ represents a hydrogen atom or hydroxyl group;
R⁴ represents CH₂—COOH, —(CH₂)₂—COOH, -methoxy-COOH, -ethoxy-COOH, -cyclopropyl-COOH, -tetrazole, -methyl-tetrazole;
each R⁵ represents fluorine or CF₃;
each R⁶ represents methyl;

or a pharmaceutically acceptable salt thereof.

In a further embodiment, the present invention provides compounds of formula (I) wherein
m is 1;
R¹ is a chlorine atom in the 4-position of the benzene ring relative to the carbon atom to which the CH₂ linking group is attached;
n is 1 or 2;
R² represents a hydrogen atom or a methyl group;
R³ represents a hydroxyl group;
R⁴ represents CH₂—COOH or —(CH₂)₂—COOH;
each R⁵ represents halogen;
q is 0;

or a pharmaceutically acceptable salt thereof.

In a further embodiment, the present invention provides compounds of the following formula:

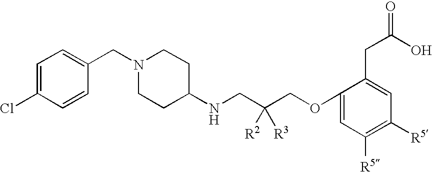

wherein
R² represents hydrogen, a methyl group;
R³ represents a hydroxyl group;
R⁵′ and R⁵″ are both hydrogen, or one of R⁵′ and R⁵″ represents fluorine or chlorine and the other of R⁵′ and R⁵″ represents hydrogen;

or a pharmaceutically acceptable salt thereof.

In another embodiment, the compounds of the invention include the following compounds:
{2-[((2S)-3-{[1-(4-chlorobenzyl)piperidin-4-yl]amino}-2-hydroxy-2-methylpropyl)oxy]-4-fluorophenyl}acetic acid,
{4-chloro-2-[((2S)-3-{[1-(4-chlorobenzyl)piperidin-4-yl]amino}-2-hydroxy-2-methylpropyl)oxy]phenyl}acetic acid,
{2-[((2S)-3-{[1-(4-chlorobenzyl)piperidin-4-yl]amino}-2-hydroxy-2-methylpropyl)oxy]phenyl}acetic acid,
3-{2-[((2S)-3-{[1-(4-chlorobenzyl)piperidin-4-yl]amino}-2-hydroxypropyl)oxy]-4-fluorophenyl}propanoic acid,
{5-chloro-2-[((2S)-3-{[1-(4-chlorobenzyl)piperidin-4-yl]amino}-2-hydroxy-2-methylpropyl)oxy]phenyl}acetic acid,
3-{2-[((2S)-3-{[1-(4-chlorobenzyl)piperidin-4-yl]amino}-2-hydroxy-2-methylpropyl)oxy]-4-fluorophenyl}propanoic acid,
3-[2-(3-{[1-(4-chlorobenzyl)piperidin-4-yl]amino}propoxy)phenyl]propanoic acid,
3-{2-[((2S)-3-{[1-(4-chlorobenzyl)piperidin-4-yl]amino}-2-hydroxy-2-methylpropyl)oxy]phenyl}propanoic acid,
{5-chloro-2-[((2S)-3-{[1-(4-chlorobenzyl)piperidin-4-yl]amino}-2-hydroxypropyl)oxy]phenyl}acetic acid,
[2-(3-{[1-(4-chlorobenzyl)piperidin-4-yl]amino}propoxy)phenyl]acetic acid,
3-{5-chloro-2-[((2S)-3-{[1-(4-chlorobenzyl)piperidin-4-yl]amino}-2-hydroxy-2-methylpropyl)oxy]phenyl}propanoic acid,
[5-chloro-2-(3-{[1-(4-chlorobenzyl)piperidin-4-yl]amino}propoxy)phenyl]acetic acid,
3-{2-[((2S)-3-{[1-(4-chlorobenzyl)piperidin-4-yl]amino}-2-hydroxy-2-methylpropyl)oxy]-5-fluorophenyl}propanoic acid,
(2-{[(2S)-3-{[1-(4-chlorobenzyl)piperidin-4-yl]amino}-2-hydroxypropyl]oxy}-4-fluorophenyl)acetic acid,
3-[2-(3-{[1-(4-chlorobenzyl)piperidin-4-yl]amino}propoxy)-4-fluorophenyl]propanoic acid,
[2-{[(2S)-3-{[1-(4-chlorobenzyl)piperidin-4-yl]amino}-2-hydroxy-2-methylpropyl]oxy}-4-(trifluoromethyl)phenyl]acetic acid,
(2-{[(2S)-3-{[1-(4-fluorobenzyl)piperidin-4-yl]amino}-2-hydroxypropyl]oxy}phenyl)acetic acid,
(2-{[(2S)-3-({1-[4-chloro-3-(trifluoromethyl)benzyl]piperidin-4-yl}amino)-2-hydroxypropyl]oxy}phenyl)acetic acid,
(2-{[(2S)-3-{[1-(3,4-difluorobenzyl)piperidin-4-yl]amino}-2-hydroxypropyl]oxy}phenyl)acetic acid,
(2-{[(2S)-3-{[1-(2,5-dimethylbenzyl)piperidin-4-yl]amino}-2-hydroxypropyl]oxy}phenyl)acetic acid,
(2-{[(2S)-3-{[1-(2-chloro-4-fluorobenzyl)piperidin-4-yl]amino}-2-hydroxypropyl]oxy}phenyl)acetic acid,
(2-{[(2S)-3-{[1-(3-chloro-4-fluorobenzyl)piperidin-4-yl]amino}-2-hydroxypropyl]oxy}phenyl)acetic acid,
(2-{[(2S)-3-{[1-(3,5-dimethylbenzyl)piperidin-4-yl]amino}-2-hydroxypropyl]oxy}phenyl)acetic acid,
(2-{[(2S)-3-{[1-(4-chloro-2-fluorobenzyl)piperidin-4-yl]amino}-2-hydroxypropyl]oxy}phenyl)acetic acid,
(2-{[(2S)-3-{[1-(4-bromobenzyl)piperidin-4-yl]amino}-2-hydroxypropyl]oxy}phenyl)acetic acid,
(2-{[(2S)-3-{[1-(4-chlorobenzyl)piperidin-4-yl]amino}-2-hydroxy-2-methylpropyl]oxy}-3,4-difluorophenyl)acetic acid,
2-(2-{[(2S)-3-{[1-(4-chlorobenzyl)piperidin-4-yl]amino}-2-hydroxypropyl]oxy}phenyl)propanoic acid,
{2[2-({[1-(4-chlorobenzyl)piperidin-4-yl]amino}methyl)-2-hydroxybutoxy]phenyl}acetic acid,
(2-{[(2S)-3-{[1-(4-chlorobenzyl)-2,5-dimethylpiperidin-4-yl]amino}-2-hydroxy-2-methylpropyl]oxy}phenyl)acetic acid,
(2-{[(2S)-3-{[1-(4-chloro-2-methylbenzyl)piperidin-4-yl]amino}-2-hydroxypropyl]oxy}phenyl)acetic acid,
2-(2-{[(2S)-3-{[1-(4-chlorobenzyl)piperidin-4-yl]amino}-2-hydroxy-2-methylpropyl]oxy}phenyl)propanoic acid,
1-(2-{[(2S)-3-{[1-(4-chlorobenzyl)piperidin-4-yl]amino}-2-hydroxypropyl]oxy}phenyl)cyclopropanecarboxylic acid,
1-(2-{[(2S)-3-{[1-(4-chlorobenzyl)piperidin-4-yl]amino}-2-hydroxy-2-methylpropyl]oxy}phenyl)cyclopropanecarboxylic acid,
(2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[(3S,4R)-1-(4-chlorobenzyl)-3-methylpiperidin-4-yl]amino}-2-hydroxy-2-methylpropyl]oxy}-4-fluorophenyl)acetic acid,
(2-{[(2S)-3-{[(3R,4R)-1-(4-chlorobenzyl)-3-methylpiperidin-4-yl]amino}-2-hydroxy-2-methylpropyl]oxy}-4-fluorophenyl)acetic acid,
2-(2-{[(2S)-3-{[1-(4-chlorobenzyl)piperidin-4-yl]amino}-2-hydroxy-2-methylpropyl]oxy}-4-fluorophenyl)propanoic acid,
2-[2-(3-{[1-(4-chlorobenzyl)piperidin-4-yl]amino}propoxy)phenoxyl]propanoic acid,
[2-(3-{[1-(4-chlorobenzyl)piperidin-4-yl]amino}propoxy)phenoxy]acetic acid,
(2-{[(2S)-3-{[1-(4-chlorobenzyl)-2,2,6,6-tetramethylpiperidin-4-yl]amino}-2-hydroxy-2-methylpropyl]oxy}-4-fluorophenyl)acetic acid,
(2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid,
3-[2-(3-{[(2R,4S,5S)-1-(4-chlorobenzyl)-2,5-dimethylpiperidin-4-yl]amino}propoxy)phenyl]propanoic acid,
3-[2-(3-{[1-(3,4-dichlorobenzyl)piperidin-4-yl]amino}propoxy)phenyl]propanoic acid,
(2-{[(2S)-3-{[1-(3,4-dichlorobenzyl)piperidin-4-yl]amino}-2-hydroxy-2-methylpropyl]oxy}-4-fluorophenyl)acetic acid,
(2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[1-(4-chlorobenzyl)-3,3-dimethylpiperidin-4-yl]amino}-2-hydroxy-2-methylpropyl]oxy}-4-fluorophenyl)acetic acid,
(2S)-1-{[1-(4-chlorobenzyl)piperidin-4-yl]amino}-3-[2-(1H-tetrazol-5-yl)phenoxy]propan-2-ol,
(2R)-1-{[1-(4-chlorobenzyl)piperidin-4-yl]amino}-2-methyl-3-[2-(1H-tetrazol-5-yl)phenoxy]propan-2-ol,
(2S)-1-{[1-(4-chlorobenzyl)piperidin-4-yl]amino}-3-[2-(1H-tetrazol-5-ylmethyl)phenoxy]propan-2-ol,
(2S)-1-{[1-(4-chlorobenzyl)piperidin-4-yl]amino}-2-methyl-3-[2-(1H-tetrazol-5-ylmethyl)phenoxy]propan-2-ol, or
(2S)-1-{[1-(4-chlorobenzyl)piperidin-4-yl]amino}-3-[5-fluoro-2-(1H-tetrazol-5-ylmethyl)phenoxy]-2-methylpropan-2-ol,
   or a pharmaceutically acceptable salt thereof.

Each exemplified compound represents a particular and independent aspect of the invention.

The present invention further provides a process for the preparation of a compound of formula (I) or a pharmaceutically acceptable salt thereof as defined above which comprises

(a) where R³ is a hydroxyl group, reacting a compound of formula

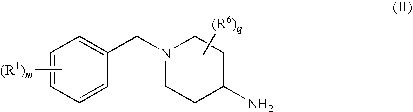

is wherein m, R¹, q and R⁶ are as defined in formula (I), with a compound of formula

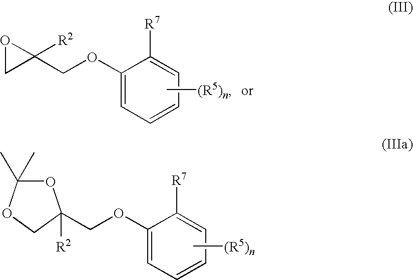

wherein R², R⁵ and n are as defined in formula (I), and R⁷ is R⁴ as defined formula (I) or a protected derivative thereof

or when R⁴ comprises a tetrazole than R⁷ may be a suitable precurser such as a nitril, thiocyanate or amide; or

(b) where R³ is a hydroxyl group, reacting a compound of formula

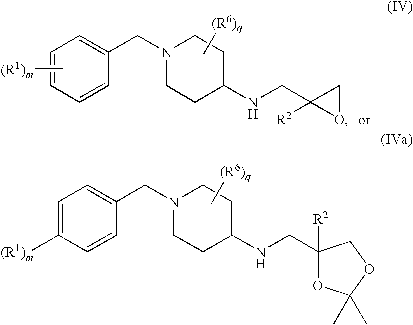

wherein m, R¹, R², q and R⁶ are as defined in formula (I), with a compound of formula

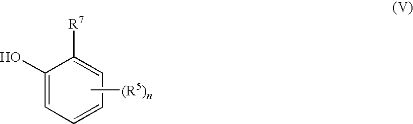

wherein R⁵ and n are as defined in formula (I), and R⁷ is R⁴ as defined formula (I) or a protected derivative thereof; or

(c) reacting a compound of formula

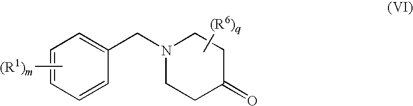

wherein m, R¹, q and R⁶ are as defined in formula (I), with a compound of formula

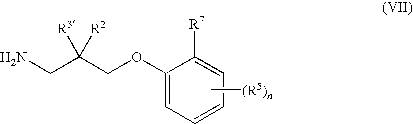

wherein R², R⁵ and n are as defined in formula (I), R³′ is R³ as defined in formula (I) or —O—P where P is a suitable protecting group, and R⁷ is R⁴ as defined formula (I) or a protected derivative thereof;

(d) reacting a compound of formula

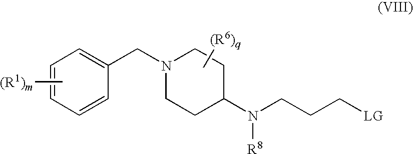

wherein m, R¹, q and R⁶ are as defined in formula (I), R⁸ is H or a suitable protecting group, and LG is a suitable leaving group, with a compound of formula

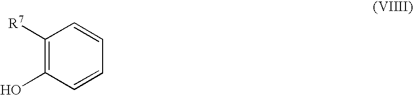

wherein R⁷ is R⁴ as defined in formula (I) or a protected derivative thereof;

and optionally after (a), (b), (c) or (d) removing any protecting groups and/or forming a pharmaceutically acceptable salt of the compound of formula (I).

As will be apparent to a person skilled in the art, when an S-enantiomer (i.e. compounds with the S configuration at the stereocentre with R² and R³ attached) is synthesised, the intermediates III, IV and VII can have the relevant S configuration to ensure the configuration is maintained in the final product.

Processes (a) to (d) may conveniently be carried out in a solvent, e.g. an organic solvent such as an alcohol (e.g. methanol or ethanol), a hydrocarbon (e.g. toluene), THF or acetonitrile at a temperature of, for example, 15° C. or above such as a temperature in the range from 20 to 120° C.

Process (b) typically requires the use of a base such as sodium hydride. Other suitable bases may be used, for example lithium diisopropylamine or lithium hexamethyldisilazide.

Process (c) typically requires the use of a suitable reducing agent such as sodium borohydride or similar mild reducing reagents (e.g. NaBH₃CN, NaBH(OAc)₃), any of which would be routine for a person skill in the art.

In process (d), the choice of a suitable leaving group LG would be routine for a person skilled in the art. A suitable leaving group may, for example, be formed by the reaction of the compound.

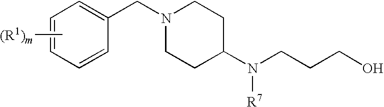

with DEAD (diethyl azodicarboxylate) and Ph₃P. However, the use of other leaving groups (e.g. Cl, Br, tosylate (4-toluenesulfonate), mesylate (methanesulfonate) are possible.

Process (d) may typically require the use of a base such as potassum carbonate or cesium carbonate, or any other appropriate base such as tertiary amines N-ethyldiisopropylamine or 1,4-diazabicyclo[2.2.2]octane (DABCO).

It will be appreciated by those skilled in the art that in the processes of the present invention certain functional groups such as carboxyl, hydroxyl, or amino groups in the starting reagents or intermediate compounds may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

For example, in the processes of the invention, R⁴ may be protected by a suitable protecting group. As an example, R⁶ maybe —C₁-C₄ alkyl-COOP′, where P′ is a suitable protecting group (e.g. methyl or ethyl). After the reaction, the ester can be hydrolysed to afford the required acid functionality (or salt thereof). However, a person skilled in the art would recognise that R⁴ may be protected by other functional groups (other than esters) which upon their removal, affords the required acid functionality (or salt thereof).

A person skilled in the art would recognise that R⁷ may take the form of any protecting group suitable for protecting secondary amines. An example of a suitable protecting group R⁷ is tert-butoxycarbonyl which can be removed using, for example, TFA.

Compounds of formulae (II), (III), (Ma), (IV), (IVa), (V), (VI), (VII), (VIII), and (VIII) are either commercially available, are well known in the literature or may be prepared easily using known techniques, for example as shown in the accompanying Examples.

Intermediates of formula (II), (V) and (VII) and salts thereof are believed to be novel and comprise an independent aspect of the invention.

Compounds of formula (I) above may be converted to a pharmaceutically acceptable salt or solvate thereof, preferably an acid addition salt such as a hydrochloride, hydrobromide, phosphate, sulfphate, acetate, ascorbate, benzoate, fumarate, hemifumarate, furoate, succinate, maleate, tartrate, citrate, oxalate, xinafoate, methanesulphonate or p-toluenesulphonate. A pharmaceutically acceptable salt also includes internal salt (zwitterionic) forms.

The compounds of formula (I) and pharmaceutically acceptable salts thereof may exist in solvated, for example hydrated, as well as unsolvated forms, and the present invention encompasses all such solvated forms.

The compounds of formula (I) are capable of existing in stereoisomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC. Alternatively, the optical isomers may be obtained by asymmetric synthesis, or by synthesis from optically active starting materials. Preferred optical isomers are the (S)-enantiomers (i.e. compounds with the S configuration at the stereocentre with R² and R³ attached).

It will be appreciated that the compounds of formula (I) and salts thereof may exist as zwitterions (internal salts). Accordingly, the representation of formula (I) and the examples of the present invention covers zwitterionic forms.

The compounds of formula (I) have activity as pharmaceuticals, and are modulators of chemokine receptor (especially CCR1 receptor) activity, and may be used in the treatment of autoimmune, inflammatory, proliferative and hyperproliferative diseases and immunologically-mediated diseases. The compounds of the present invention may also have beneficial potency, selectivity and/or pharmacokinetic properties. For example, example compounds of the present invention may have low activity against the human ether-a-go-go-related-gene (hERG)-encoded potassium channel. In this regard, drugs blocking hERG and consequently restoration of the negative cell potential by K⁺ efflux, can cause a prolongation of the QT interval, leading to an acquired long QT syndrome (LQT) [M. C. Sanguinetti, C Jiang, M. E. Curran, M. T. Keating, Cell 1995, 81, 299-307; and K. Finlayson et al., Eur. J. Pharm. 2004, 500, 129-142]. This, however, may induce a potentially fatal arrythmia, known as torsade de points (TdP) [W. Haferkamp et al., Eur. Heart J. 2000, 21, 1216-1331]. New chemical entities, if not intended for cardiovascular use, which are lacking effect on cardiac channels, and the hERG channel in particular, will therefore provide an improved safety profile and so gain a therapeutical and regulatory advantage over drugs with QT prolonging effects. Kiss et al (Assay Drug Dev. Technol. 2003, 1,127-135) describe a method of assaying compounds for their ability to inhibit ion channel activity such as hERG.

A compound of the invention, or a pharmaceutically acceptable salt thereof; can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all seventies, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and to iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral , fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis , inflammatory and auto-immune cardiomyopathies including myocardial sarcoid; ischaemic reperfusion injuries; endocarditis, valvulitis, and aortitis -including infective (for example syphilitic); vasculitides; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; and,

15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Thus, in a further aspect, the present invention provides a compound of formula (I) or a pharmaceutically-acceptable salt or solvate thereof, as hereinbefore defined for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In a further aspect, the present invention provides a method of treating a respiratory disease in a patient suffering from, or at risk of, said disease, which comprises administering to the patient a therapeutically effective amount of a compound of formula (I) or a pharmaceutically acceptable salt thereof, as hereinbefore defined.

In a further aspect, the present invention provides the use of a compound of formula (I) or a pharmaceutically-acceptable salt thereof, as hereinbefore defined in the manufacture of a medicament for use in treating a respiratory disease.

In a further aspect, the present invention provides a method of treating an airways disease in a patient suffering from, or at risk of, said disease, which comprises administering to the patient a therapeutically effective amount of a compound of formula (I) or a pharmaceutically acceptable salt thereof, as hereinbefore defined.

In a further aspect, the present invention provides the use of a compound of formula (I) or a pharmaceutically-acceptable salt thereof, as hereinbefore defined in the manufacture of a medicament for use in treating an airways disease.

In a further aspect, the present invention provides a method of treating an inflammatory disease in a patient suffering from, or at risk of, said disease, which comprises administering to the patient a therapeutically effective amount of a compound of formula (I), or a pharmaceutically acceptable salt thereof, as hereinbefore defined.

In a further aspect, present invention provides the use of a compound of formula (I) or a pharmaceutically-acceptable salt thereof, as hereinbefore defined in the manufacture of a medicament for use in treating an inflammatory disease.

In a further aspect, the present invention provides a method of treating asthma or chronic is obstructive pulmonary disease in a patient suffering from, or at risk of, said disease, which comprises administering to the patient a therapeutically effective amount of a compound of formula (I) or a pharmaceutically acceptable salt thereof, as hereinbefore defined.

In a further aspect, the present invention provides the use of a compound of formula (I) or a pharmaceutically-acceptable salt thereof, as hereinbefore defined in the manufacture of a medicament for use in treating asthma or chronic obstructive pulmonary disease.

In a further aspect, the present invention provides the use of a compound of formula (I) or a pharmaceutically-acceptable salt thereof, as hereinbefore defined in the manufacture of a medicament for the treatment of human diseases or conditions in which modulation of CCR1 activity is beneficial.

In a further aspect, the present invention provides for an agent for the treatment of an inflammatory disease, an airways disease, asthma or chronic obstructive pulmonary disease, which comprises as active ingredient a compound of formula I as hereinbefore defined.

For the above-mentioned therapeutic uses the dosage administered will, of course, vary with the compound employed, the mode of administration, the treatment desired and the disorder indicated. The daily dosage of the compound of formula (I) may be in the range from 0.001 mg/kg to 30 mg/kg.

The compound of formula (I) and pharmaceutically acceptable salts thereof may be used on their own but will generally be administered in the form of a pharmaceutical composition in which the formula (I) compound or salt thereof (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt thereof, as hereinbefore defined, in association with a pharmaceutically acceptable adjuvant, diluent or carrier.

The invention further provides a process for the preparation of a pharmaceutical composition of the invention, which comprises mixing a compound of formula (I), or a pharmaceutically acceptable salt thereof, as hereinbefore defined, with a pharmaceutically acceptable adjuvant, diluent or carrier.

The invention further provides a pharmaceutical composition comprising a compound of formula (I), or a pharmaceutically acceptable salt thereof, for use in treatment of an inflammatory disease, an airways disease, asthma or chronic obstructive pulmonary disease,

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax I1-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), is (xxvi) agent modulating the activity of purinergic receptors such as P2×7; or i(xxvii) inhibitor of transcription factor activation such as NFkB, API, or STATS.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), \ddot{N}-(3-ethynylphenyl)-6,7-bis(2-methoxyethoxy)quinazolin-4-amine (erlotinib, OSI-774) or 6-acrylamido-\ddot{N}-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

The following abbreviations are used:

### APCI-MS Atmospheric Pressure Chemical Ionisation Mass Spectroscopy;

### DIEA N,N-Diisopropylethylamine;

### DMSO Dimethylsulfoxide;

### HPLC High Performance Liquid Chromatography;

### LC/MS Liquid Column Chromatography/Mass Spectroscopy;

TFA Trifluoroacetic acid;

### THF Tetrahydrofuran

### General Methods

¹H NMR and ¹³C NMR. spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H}1.95 ppm) or methanol-d₄ (δ_{H} 3.31 ppm) were used as internal references. Flash chromatography was carried out using silica gel (0.040-0.063 mm, Merck). Unless stated otherwise, starting materials were commercially available. All solvents and commercial reagents were of laboratory grade and were used as received.

The following method was used for LC/MS analysis:

Instrument Agilent 1100; Column Waters Symmetry 2.1×30 mm; Mass APCI; Flow rate 0.7 ml/min; Wavelength 254 nm; Solvent A: water+0.1% TFA; Solvent B: acetonitrile+0.1% TFA; Gradient 15-95%/B 2.7 min, 95% B 0.3 min.

The following method was used for LC analysis:

Method A. Instrument Agilent 1100; Column: Kromasil C18 100×3 mm, 5μ particle size,

Solvent A: 0.1% TFA/water, Solvent B: 0.08% TFA/acetonitrile Flow: 1 ml/min, Gradient 10-100% B 20 min, 100% B1 min. Absorption was measured at 220, 254 and 280 nm.

Method B. Instrument Agilent 1100; Column: XTerra C8, 100×3 mm, 5μ a particle size,

Solvent A: 15 mM NH₃/water, Solvent B: acetonitrile Flow: 1 ml/min, Gradient 10-100% B 20 min, 100% B1 min. Absorption was measured at 220, 254 and 280 nm.

Starting material: 1-(4-Chlorobenzyl)piperidin-4-amine was prepared according to the procedure described in WO 0198273, page 34.

### EXAMPLE 1

### {2-[((2S)-3-{[1-(4-Chlorobenzyl)piperidin-4-yl]amino}-2-hydroxy-2-methylpropyl)oxy]-4-fluorophenyl}acetic acid bis(trifluoroacetate) (salt)

1-(4-Chlorobenzyl)piperidin-4-amine (39 mg, 170 μmol) and methyl {4-fluoro-2-[(2S)-oxiran-2-ylmethoxy]phenyl}acetate (1a, 44 mg, 170 μmol) were dissolved in ethanol (3.0 ml) and the mixture was stirred overnight at 80° C. Then aqueous 2 M NaOH (1 ml) was added and stirring was continued at 80° C. for 30 min. After cooling to room temperature the mixture was acidified with trifluoroacetic acid and concentrated under reduced pressure. The crude product was purified by reverse phase HPLC (Kromasil column, 100-5-C₁₈, 250×20 mm) using acetonitrile (0.1% TFA) and H₂O (0.1% TFA). After freeze-drying, 51 mg (43%) of the title compound 1 was obtained as a colourless powder.

¹H NMR. (400 MHz, CD₃OD) δ 7.56 (s, 4H), 7.22 (t, J=7.5 Hz, 1H), 6.78 (dd, J=10.8, 2.3 Hz, 1H), 6.69 (td, J=8.4, 2.4 Hz, 1H), 4.33 (s, 2H), 3.97 (d, J=9.4 Hz, 1H), 3.91 (d, J=9.5 Hz, 1H), 3.64-3.43 (m, 5H), 3.38 (d, J=12.7 Hz, 1H), 3.16 (d, J=12.8 Hz, 1H), 3.11 (t, J=13.3 Hz, 2H), 2.43 (m, 2H), 2.05 (m, 2H), 1.43 (s, 3H).

APCI-MS m/z: 465.1 [MH⁺].

LC (method A) rt=5.91 min. 96.6% UV 220 nm.

LC (method B) rt=5.04 min.>99% UV 220 nm.

### Methyl {4-fluoro-2[(2S)-oxiran-2-ylmethoxy]phenyl}acetate (1a)

A mixture of methyl (4-fluoro-2-hydroxyphenyl)acetate (1b, 37 mg, 200 μmol, [(2S)-2-methyloxiran-2-yl]methyl 3-nitrobenzenesulfonate (55 mg, 200 μmol) and cesium carbonate (99 mg, 300 μmol) in DMF (1.5 ml) was stirred overnight at room temperature and then diluted with water (25 ml), extracted twice with ethyl acetate (25 ml) and dried over sodium sulfate. Evaporation of the solvent afforded 44 mg (86%) of the subtitled compound 1a as a brownish oil.

¹H NMR (400 MHz, CDCl₃) δ 7.13 (dd, J=8.2, 6.7 Hz, 1H), 6.64 (td, J=8.3, 2.4 Hz, 1H), 6.59 (dd, J=10.6, 2.4 Hz, 1H), 4.03 (d, J=10.3 Hz, 1H), 3.90 (d, J=10.3 Hz, 1H), 3.70 (s, 3H), 3.61 (s, 2H), 2.86 (d, J=4.7 Hz, 1H), 2.73 (d, J=4.7 Hz, 1H), 1.47 (s, 3H).

### Methyl (4-fluoro-2-hydroxyphenyl)acetate (1b)

To a solution of (4-fluoro-2-hydroxybenzyl)(triphenyl)phosphonium bromide (1c, 934 mg, 2.00 mmol) and triethylamine (607 mg, 0.84 ml, 6.0 mmol) in dichloromethane (10 ml) was added methyl chloroformiate (189 mg, 2.00 mmol), dissolved in dichlomethane (2 ml). The mixture was stirred over night and then poured into an aqueous phosphate buffer (pH=7). The aqueous mixture was extracted twice with ethyl acetate and the combined organic phases were dried over sodium sulfate. Evaporation of the solvent and flash chromatography of the crude afforded 127 mg (35%) of the subtitled compound 1b as yellow oil.

¹H NMR (400 MHz, CDCl₃) δ 7.79 (s, 1H), 7.03 (dd, J=8.2, 6.5 Hz, 1H), 6.68 (dd, J=10.0, 2.6 Hz, 1H), 6.60 (td, J=8.3, 2.6 Hz, 1H), 3.77 (s, 3H), 3.65 (s, 2H).

### (4-Fluoro-2-hydroxybenzyl)(triphenyl)phosphonium bromide (1c)

The subtitled compound 1c was prepared according to the protocol described by J. Guillaumel et al., Eur. J. Med. Chem. 1983, 18 (5), 431-436, starting from 5-fluoro-2-(hydroxymethyl)phenol (1d, 284 mg, 2.00 mmol) and triphenylphosphine hydrobromide (686 mg, 2.00 mmol). 925 mg (99%) of 1c were obtained as a white solid.

¹H NMR (400 MHz, CDCl₃) δ 7.75 (m, 3H), 7.61-7.51 (m, 12H), 7.08 (dd, J=10.4, 2.0 Hz, 1H), 6.92 (ddd, J=8.6, 6.4, 2.5 Hz, 1H), 6.32 (td, J=8.2, 2.3 Hz, 1H), 4.58 (s, 1H), 4.54 (s, 1H).

### 5-Fluoro-2-(hydroxymethyl)phenol (1d)

To an ice bath-cooled solution of lithium aluminiumhydride (1 M in THF, 25 ml, 25 mmol) a solution of 4-fluoro-2-hydroxybenzoic acid (1.56 g, 10.0 mmol) in THF (10 ml) was added drop-wise. The mixture was allowed to warm up to room temperature and after stirring over night cooled with an ice bath and quenched carefully with aqueous 2M HCl (approx. 30 ml). The mixture was extracted twice with ethyl acetate, the combined organic phases were washed with aqueous 1 M sodium bicarbonate (50 ml) and dried over sodium sulfate. Evaporation of the solvent afforded 1.37 g (96%) of 1d as a brown solid.

¹H NMR (400 MHz, CDCl₃) δ 7.58 (br.s, 1H), 6.98 (dd, J=8.2, 6.5 Hz, 1H), 6.63 (dd, J=10.2, 2.5 Hz, 1H), 6.56 (td, J=8.3, 2.5 Hz, 1H), 4.85 (s, 2H), 2.05 (s, 1H).

### EXAMPLE 2

### {4-Chloro-2-[((2S)-3-{[1-(4-chlorobenzyl)piperidin-4-yl]amino}-2-hydroxy-2-methylpropyl)oxy]phenyl}acetic acid bis(trifluoroacetate) (salt)

The titled compound was prepared according to the procedure described in Example 1 starting from 1-(4-chlorobenzyl)piperidin-4-amine (39 mg, 170 μmol) and methyl {4-chloro-2-[(2S)-2-methyloxiran-2-ylmethoxy]phenyl}acetate (2a, 47 mg, 170 μmol). Yield: 52 mg (43%) as a colourless powder.

¹H NMR (400 MHz, CD₃OD) δ 7.53-7.47 (m, 4H), 7.21 (d, J=8.0 Hz, 1H), 6.99 (m, 2H), 4.31 (s, 2H), 3.98 (d, J=9.4 Hz, 1H), 3.92 (d, J=9.4 Hz, 1H), 3.64-3.40 (m, 5H), 3.37 (d, J=12.7 Hz, 1H), 3.15 (d, J=12.8 Hz, 1H), 3.08 (t, J=12.7 Hz, 2H), 2.42 (d, J=13.4 Hz, 2H), 2.03 (m, 2H), 1.43 (s, 3H).

APCI-MS m/z: 481.1, 483.1 [MH⁺].

### Methyl (4-chloro-2-{[(2S)-2-methyloxiran-2-yl]methoxy}phenyl)acetate (2a)

The subtitled compound was prepared according to the protocol described for 1a starting from methyl (4-chloro-2-hydroxyphenyl)acetate (2b, 40 mg, 200 μmol) and (25)-2-methyloxiran-2-ylmethyl 3-nitrobenzenesulfonate (55 mg, 200 mol). Yield: 47 mg (87%) as a brown oil.

¹H NMR (400 MHz, CDCl₃) δ 7.11 (s, 1H), 6.93 (dd, J=8.0, 1.9 Hz, 1H), 6.84 (m, 1H), 4.05 (d, J=10.3 Hz, 1H), 3.91 (d, J=10.3 Hz, 1H), 3.70 (s, 3H), 3.62 (m, 2H), 2.86 (d, J=4.7 Hz, 1H), 2.73 (d, J=4.8 Hz, 1H), 1.47 (s, 3H).

### Methyl (4-chloro-2-hydroxyphenyl)acetate (2b)

The subtitled compound was prepared according to the protocol described for 1b from (4-chloro-2-hydroxybenzyl)(triphenyl)phosphonium bromide (2c, 968 mg, 2.00 mmol). 138 mg (34%) of 2b was obtained as yellow oil.

¹H NMR (400 MHz, CDCl₃) δ 7.69 (br. s, 1H), 7.01 (d, J=8.1 Hz, 1H), 6.95 (d, J=2.0 Hz, 1H), 6.87 (dd, J=8.1, 2.0 Hz, 1H), 3.77 (s, 3H), 3.65 (s, 2H).

### (4-Chloro-2-hydroxybenzyl)(triphenyl)phosphonium bromide (2c)

The subtitled compound 1c was prepared according to the protocol described by J. Guillaumel et al., Eur. J. Med. Chem. 1983, 18 (5), 431-436, from 5-chloro-2-(hydroxymethyl)phenol (1d, 317 mg, 2.00 mmol) and triphenylphosphine hydrobromide (686 mg, 2.00 mmol). 998 mg (quanitative) of 2c was obtained as brown oil.

¹H NMR (400 MHz, CDCl₃) δ 7.80-7.70 (m, 3H), 7.62-7.49 (m, 12H), 7.28 (m, 2H), 6.91 (dd, J=8.2, 2.7 Hz, 1H), 6.58 (dd, J=8.1, 1.2 Hz, 1H), 4.57 (d, J=13.6 Hz, 2H).

### 5-Chloro-2-(hydroxymethyl)phenol (2d)

The subtitled compound was prepared according to the protocol described for 1d from 4-chloro-2-hydroxybenzoic acid (1.72 g, 10.0 mmol). 1.28 g (81%) of 2d was obtained as brown solid.

¹H NMR (400 MHz, CDCl₃) δ 7.53 (br. s, 1H), 6.95 (d, J=8.0 Hz, 2H), 6.84 (dd, J=8.0, 2.0 Hz, 1H).

### EXAMPLE 3

### {2-[((2S)-3-{[1-(4-Chlorobenzyl)piperidin-4-yl]amino}-2-hydroxy-2-methylpropyl)oxy]phenyl}acetic acid bis(trifluoroacetate) (salt)

To a solution of methyl {2-[((28)-3-{[1-(4-chlorobenzyl)piperidin-4-yl]amino}-2-hydroxy-2-methylpropyl)oxy]phenyl}acetate (3a, 125 mg, 0.27 mmol) in THF (3 ml) was added aqueous NaOH (360 mg in 1.5 ml of H₂O). The reaction mixture was stirred at 75° C. for 3 h, cooled to 0° C. and the pH was adjusted to 2 by addition of aqueous CF₃CO₂H. The volatiles were removed in vacuo and the residue was purified by reverse phase HPLC (10-75% CH₃CN in H₂O containing 0.1% CF₃CO₂H) to give the titled compound (120 mg, 66%). ¹H-NMR (CDCl₃, 400 MHz): δ 7.50 (s, 4H); 7.30-7.21 (m, 2H); 6.98-6.92 (m, 2H); 4.31 (s, 2H); 3.98 (d, J=9.5 Hz, 1H); 3.93 (d, J=9.5 Hz, 1H); 3.65-3.56 (m, 4H); 3.44 (m, 1H); 3.38 (d, J=12.7 Hz, 1H); 3.17 (d, J=12.7 Hz, 1H); 3.08 (t, J=12.9 Hz, 2H); 2.42 (d, J=13.8 Hz, 2H); 2.05 (m, 2H); 1.42 (s, 3H).

APCI-MS: m/z 447 [MH¹]

### Methyl {2-[((2S)-3-{[1-(4-chlorobenzyl)piperidin-4-yl]amino}-2-hydroxy-2-methylpropyl)oxy]phenyl}acetate (3a)

Methyl (2-{[(2S)-2-methyloxiran-2-yl]methoxy}phenyl)acetate (3b, 88 mg, 0.37 mmol) and 1-(4-chlorobenzyl)piperidin-4-amine (83 mg, 0.37 mmol) were taken into methanol (3 ml) and stirred at 75° C. over night. The volatiles were removed in vacuo and the residue was purified by flash chromatography (0-2% methanol in dichloromethane containing 0.2% NH₄OH) to give the subtitled compound 3a (134 mg, 79%).

¹H-NMR (CDCl₃, 400 MHz): δ 7.30-7.18 (m, 6H); 6.94 (t, J=7.3 Hz, 1H); 6.87 (d, J=8.2 Hz, 1H); 3.86 (d, J=8.9 Hz, 1H); 3.79 (d, J=8.9 Hz, 1H); 3.68 (s, 3H); 3.63 (s, 2H); 3.45 (s, 2H); 2.94 (d, J=12.2 Hz, 1H); 2.80 (d, J=11.4 Hz, 2H); 2.56 (d, J=12.2 Hz, 1H); 2.43 (m, 1H); 1.99 (t, J=11.4 Hz, 2H); 1.85 (br. d, J=10.7 Hz, 2H); 1.36 (m, 2H); 1.29 (s, 3H).

APCI-MS: m/z 461 (MH⁺).

Methyl (2-{[(2S)-2-methyloxiran-2-yl]methoxy}phenyl)acetate (3b)

A mixture of [(28)-2-methyloxiran-2-yl]methyl 3-nitrobenzenesulfonate (137 mg, 0.5 mmol), methyl (2-hydroxyphenyl)acetate (3c) (83 mg, 0.5 mmol) and Cs₂CO₃ (212 mg, 0.65 mmol) in DMF (5 ml) was stirred at room temperature overnight. The reaction mixture was partitioned between ethyl acetate and H₂O. The organic layer was dried over Na₂SO₄, filtered and concentrated in vacuo. The residue was purified by flash chromatography (0-30% ethyl acetate in petroleum ether 40-60° C.) to give the subtitled compound 3b (92 mg, 78%).

¹H-NMR. (CDCl₃, 400 MHz): δ 7.28-7.19 (m, 2H); 6.95 (t, J=7.4 Hz, 1H); 6.84 (d, J=8.1 Hz, 1H); 4.03 (d, J=10.3 Hz, 1H); 3.95 (d, J=10.3 Hz, 1H); 3.75 (s, 3H); 3.68 (s, 2H); 2.87 (d, J=4.7 Hz, 1H); 2.73 (d, J=4.7 Hz, 1H); 1.24 (s, 3H).

### EXAMPLE 4

### 3-{2-[((2S)-3-{[1-(4-Chlorobenzyl)piperidin-4-yl]amino}-2-hydroxypropyl)oxy]-4-fluorophenyl}propanoic acid bis(trifluoroacetate) (salt)

Methyl 3-{2-[((2S)-3-{[1-(4-chlorobenzyl)piperidin-4-yl]amino}-2-hydroxypropyl)oxy]-4-fluorophenyl}propanoate (4a) (85 mg, 0.177 mmol) was dissolved in THF (3 ml), aqueous NaOH (165 mg NaOH in 1.5 ml H₂O) was added and the reaction mixture was stirred at 80° C. for 2.5 h, cooled to 0° C. and the pH was adjusted to 2 by the addition of aqueous CF₃CO₂H. The volatiles were removed in vacuo and the residue was purified by reverse is phase HPLC (10-75% CH₃CN in H₂O containing 0.1% CF₃CO₂H) to give the titled compound (90 mg, 73%).

¹H-NMR (CD₃OD, 400 MHz): δ 7.50 (s, 4H); 7.16 (dd, J=6.9, 8.2 Hz, 1H); 6.75 (dd, J=2.3, 10.9 Hz, 0.1H); 6.67-6.60 (m, 1H); 4.30 (m, 3H); 4.08 (dd, J=4.5, 9.9 Hz, 1H); 4.01 (dd; J=5.5, 9.9 Hz, 1H); 3.60-3.45 (m, 3H); 3.40 (dd, J=2.8, 12.6 Hz, 1H); 3.26 (m, 1H); 3.02 (br.t, 2H); 2.90 (t, J=7.7 Hz, 2H); 2.61-2.48 (m, 2H); 2.40 (br,t, 2H); 2.07-2.92 (m, 2H).

APCI-MS: m/z 465 (MH⁺).

### Methyl 3-{2-[((2S)-3-{[1-(4-chlorobenzyl)piperidin-4-yl]amino}-2-hydroxypropyl)oxy]-4-fluorophenyl}propanoate (4a)

Methyl 3-{4-fluoro-2-[(2S)-oxiran-2-ylmethoxy]phenyl}propanoate (4b) (100 mg, 0.393 mmol) and 1-(4-chlorobenzyl)piperidin-4-amine (88 mg, 0.393 mmol) were taken into methanol (3 ml) and the solution stirred at 80° C. overnight. The volatiles were removed in vacuo and the residue was purified by flash chromatography (0-2.5% methanol in dichloromethane containing 0.2% NH₄OH) to give the subtitled compound 4a (100 mg, 53%).

¹H-NMR (DMSO-d₆, 400 MHz): δ 7.35 (m, 2H); 7.29 (m, 2H); 7.13 (t, J=7.6 Hz, 1H); 6.84 (dd, J=2.3, 11.4 Hz, 1H); 6.68-6.60 (m, 1H); 4.99 (br.s, 1H); 3.99-3.80 (m, 3H); 3.57 (s, 3H); 3.41 (s, 2H); 2.77 (t, J=7.7 Hz, 2H); 2.74-2.51 (m, 6H); 2.38 (m, 1H); 1.95 (t, J=10.5 Hz, 2H); 1.73 (m, 2H); 1.20 (m, 2H).

APCI-MS: m/z 479 (MH⁺).

### Methyl 3-{4-fluoro-2-[(2S)-oxiran-2-ylmethoxy]-phenyl}propanoate (4b)

A mixture of (2S)-oxiran-2-ylmethyl 3-nitrobenzenesulfonate (130 mg, 0.5 mmol), methyl 3-(4-fluoro-2-hydroxyphenyl)propanoate (99 mg, 0.5 mmol) and Cs₂CO₃ (196 mg, 0.6 to mmol) in DMF (3 ml) was stirred at room temperature over night. The reaction mixture was partitioned between ethyl acetate and water. The organic layer was dried over Na₂SO₄, filtered and concentrated in vacuo. The residue was purified by flash chromatography (0.20% ethyl acetate in petroleum ether 40-60° C.) to give the subtitled compound (105 mg, 83%).

¹H-NMR (CDCl₃, 400 MHz): δ 7.10 (t, J=7.5 Hz, 1H); 6.64-6.55 (m, 2H); 4.26 (dd, J=2.8, 11.1 Hz, 1H); 3.93 (dd, J=5.6, 11.1 Hz, 1H); 3.69 (s, 3H); 3.39 (m, 1H); 2.96-2.90 (m, 3H); 2.78 (dd, J=2.7, 4.9 Hz, 1H); 2.60 (t, J=7.7 Hz, 2H).

### EXAMPLE 5

### {5-Chloro-2-[((2S)-3-{[1-(4-chlorobenzyl)piperidin-4-yl]amino}-2-hydroxy-2-methylpropyl)oxy]phenyl}acetic acid bis(trifluoroacetate) (salt)

The titled compound was prepared according to the procedure described in Example 1 starting from 1-(4-chlorobenzyl)piperidin-4-amine (103 mg, 460 μmol) and methyl {5-chloro-2[(2S)-2-methyloxiran-2-ylmethoxy]phenyl}acetate (5a, 123 mg, 460 μmol.

Yield: 220 mg (68%) as a colourless powder.

¹H NMR (400 MHz, CD₃OD) δ 7.50 (m, 4H), 7.26 (m, 2H), 6.95 (m, 1H), 4.33 (s, 2H), 3.97 (d, J=9.4 Hz, 1H), 3.91 (d, J=9.5 Hz, 1H), 3.65-3.41 (m, 5H), 3.37 (d, J=12.7 Hz, 1H), 3.16 (d, J=12.8 Hz, 1H), 3.11 (t, J=13.8 Hz, 2H), 2.43 (d, J=13.4 Hz, 2H), 2.04 (m, 2H), 1.42 (s, 3H).

APCI-MS m/z: 481.1, 483.1 [MH⁺].

### Methyl (5-chloro-2-{[(2S)-2-methyloxiran-2-yl]methoxy}phenyl)acetate (5a)

The subtitled compound was prepared according to the protocol described for 1a starting from methyl (4-chloro-2-hydroxyphenyl)acetate (111 mg, 550 μmol) and (2S)-2-methyloxiran-2-ylmethyl 3-nitrobenzenesulfonate (151 mg, 550 mol). Yield: 148 mg (83%) as brown oil.

¹H NMR (400 MHz, CDCl₃) δ 7.21 (d, J=2.6 Hz, 1H), 7.18 (m, 2H), 4.03 (d, J=10.4 Hz, 1H), 3.90 (d, J=10.3 Hz, 1H), 3.70 (s, 3H), 3.62 (s, 2H), 2.85 (d, J=4.7 Hz, 1H), 2.72 (d, J=4.8 Hz, 1H), 1.46 (s, 3H).

### Methyl (5-chloro-2-hydroxyphenyl)acetate (5b)

The subtitled compound was prepared according to the protocol described for 1b from (5-chloro-2-hydroxybenzyl)(triphenyl)phosphonium bromide (5c, 483 mg, 1.00 mmol).

Yield: 125 mg (63%) as yellow oil.

¹H NMR (400 MHz, CDCl₃) δ 7.32 (br. s, 1H), 7.16 (dd, J=8.6, 2.6 Hz, 1H), 7.09 (d, J=2.5 Hz, 1H), 6.88 (d, J=8.6 Hz, 1H), 3.77 (s, 3H), 3.65 (s, 2H).

### (5-Chloro-2-hydroxybenzyl)(triphenyl)phosphonium bromide (5c)

The subtitled compound was prepared according to the protocol described by J. Guillaumel et al., Eur. J. Med. Chem. 1983, 18 (5), 431-436, from 5-chloro-2-(hydroxymethyl)phenol (5d, 1.59 g, 10 mmol) and triphenylphosphine hydrobromide (3.43 g, 10 mmol). 4.95 g (quanitative) of 5c was obtained as a colourless powder.

¹H NMR (400 MHz, CDCl₃) δ 7.79-7.70 (m, 3H), 7.62-7.51 (m, 12H), 7.24 (d, J=8.8 Hz, 1H), 6.88 (m, 1H), 6.75 (t, J=2.5 Hz, 1H), 4.68 (d, J=13.7 Hz, 2H).

### EXAMPLE 6

### 3-{2-[((2S)-3-{[1-(4-chlorobenzyl)piperidin-4-yl]amino}-2-hydroxy-2-methylpropyl)oxy]-4-fluorophenyl}propanoic acid bis(trifluoroacetate) (salt)

Methyl 3-{2-[((2S)-3-{[1-(4-chlorobenzyl)piperidin-4-yl]amino}-2-hydroxy-2-methylpropyl)oxy]-4-fluorophenyl}propanoate (6a) (110 mg, 0.223 mmol) was dissolved in THF (3 ml), aqueous NaOH (165 mg NaOH in 1.5 ml H₂O) was added and the reaction mixture stirred at 80° C. for 1.5 h, cooled to 0° C., and pH adjusted to 2 by addition of aqueous CF₃CO₂H. The volatiles were removed in vacuo and the residue was purified by reverse phase HPLC (10-75% CH₃CN in H₂O containing 0.1% CF₃CO₂H) to give the titled compound (130 mg, 82%).

¹H-NMR. (CD₃OD, 400 MHz): S 7.51 (s, 4H); 7.17 (dd, J=7.0, 8.3 Hz, 1H); 6.75 (dd, J=2.3, 10.9 Hz, 1H); 6.68-6.61 (m, 1H); 4.37 (s, 2H); 3.97 (d, J=9.4 Hz, 1H); 3.92 (d, J=9.4 Hz, 1H); 3.61 (br.d, J=12.3 Hz, 2H); 3.50 (m, 1H); 3.41 (d, J=12.7 Hz, 1H); 3.22 (d, J=12.7 Hz, 1H); 3.12 (t, J=12.5 Hz, 2H); 2.90 (t, J=7.7 Hz, 2H); 2.85 (m, 2H); 2.43 (m, 2H); 2.06 (m, 2H); 1.45 (s, 3H).

APCI-MS: m/z 493 (MH⁺).

### Methyl 3-{2-[((2S)-3-{[1-(4-chlorobenzyl)piperidin-4-yl]amino}-2-hydroxy-2-methylpropyl)oxy]-4-fluorophenyl}propanoate (6a)

Methyl 3-(4-fluoro-2-{[(2S)-2-methyloxiran-2-yl]-methoxy}phenyl) propanoate (6b) (80 mg, 0.298 mmol) and 1-(4-chlorobenzyl)piperidin-4-amine (67 mg, 0.298 mmol) were taken into methanol (3 ml) and stirred at 80° C. overnight. The volatiles were removed in vacuo and the residue was purified by silica gel flash chromatography (0-2% methanol in dichloromethane containing 0.2% NH₄OH) to give the subtitled compound 6a (125 mg, 85%).

¹H-NMR (DMSO-d₆, 400 MHz): δ 7.35 (m, 2H); 7.28 (m, 2H); 7.13 (t, J=7.6 Hz, 1H); 6.81 (dd, J=2.4, 11.3 Hz, 1H); 6.65 (m, 1); 4.68 (br.s, 1H); 3.86 (d, J=9.0 Hz, 1H); 3.70 (d, J=9.0 Hz, 1H); 3.58 (s, 3H); 3.40 (s, 2H); 2.78 (t, J=7.5 Hz, 2H); 2.72-2.52 (m, 6H); 2.31 (m, 1H); 1.92 (m, 2H); 1.73 (m, 2H); 1.27 (m, 2H); 1.19 (s, 3H).

APCI-MS: m/z 493 (MH⁺).

### Methyl 3-(4-fluoro-2-{[(2S)-2-methyloxiran-2-yl]methoxy}phenyl)propanoate (6b)

A mixture of methyl 3-(4-fluoro-2-hydroxyphenyl)propanoate (6c) (99 mg, 0.5 mmol), [(2S)-2-methyloxiran-2-yl]methyl 3-nitrobenzenesulfonate (137 mg, 0.5 mmol) and Cs₂CO₃ (196 mg, 0.6 mmol) in DMF (3 ml) was stirred at room temperature over night. The reaction mixture was partitioned between ethyl acetate and H₂O, and the organic layer was dried over Na₂SO₄, filtered and concentrated in vacuo. The residue was purified by silica gel flash chromatography (0-20% ethyl acetate in petroleum spirit 40-60° C.) to give the subtitled compound (6b) (90 mg, 67%).

¹H-NMR (CDCl₃, 300 MHz): δ 7.10 (dd, J=6.9, 8.1 Hz, 1H); 6.64-6.53 (m, 2H); 4.05 (d, J=10.4 Hz, 1H); 3.90 (d, J=10.4 Hz, 1H); 3.67 (s, 3H); 2.96-2.86 (m, 3H); 2.76 (d, J=4.8 Hz, 1H); 2,60 (t, J=7.7 Hz, 2H); 1.48 (s, 3H).

### Methyl 3-(4-fluoro-2-hydroxyphenyl)propanoate (6c)

Methyl (2E)-3-(4-fluoro-2-hydroxyphenyl)acrylate (350 mg, 2.5 mmol) was taken into ethyl acetate (12 ml), Pt/C (5%, 70 mg) was added and the resulting mixture was hydrogenated at atmospheric pressure and at room temperature for 8 h. The catalyst was filtered off and the filtrate was concentrated in vacuo to give the subtitled compound 6c (327 mg, 93%).

¹H-NMR (DMSO-d₆, 400 MHz): δ 9.99 (br.s, 1H); 7.02 (t, J=7.7 Hz, 1H); 6.54 (dd, J=2.6, 10.8 Hz, 1H); 6.51-6.45 (m, 1H); 3.59 (s, 3H); 2.70 (t, J=7.6 Hz, 2H); 2.50 (m, 2H).

### Methyl (2E)-3-(4-fluoro-2-hydroxyphenyl)acrylate (6d)

A solution of 4-fluoro-2-hydroxybenzaldehyde (420 mg, 3 mmol) in THF (9 ml) was cooled in an ice-water bath. To this cold solution, a solution of methyl (triphenylphosphoranylidene)acetate (1 g, 3 mmol) in dichloromethane (6 ml) was added dropwise. After addition was completed, the ice bath was removed and the reaction mixture was stirred at room temperature for 24 h. The volatiles were removed in vacuo and the residue was purified by flash chromatography (0-30% ethyl acetate in petroleum spirit 40-60° C.) to give the subtitled compound (6d) (545 mg, 93%). ¹H-NMR (DMSO-d₆, 400 MHz): δ 11.00 (br.s, 1H); 7.81 (d, J=16.1 Hz, 1H); 7.67 (t, J=7.9 Hz, 1H); 6.71-6.54 (m, 2H); 6.59 (d, J=16.1 Hz, 1H); 3.68 (s, 3H).

### EXAMPLE 7

### 3-[2-(3-{[1-(4-Chlorobenzyl)piperidin-4-yl]amino}propoxy)phenyl]propanoic acid bis(trifluoroacetate) (salt)

To a solution of tert-butyl[1-(4-chlorobenzyl)piperidin-4-yl](3-hydroxypropyl)carbamate (7a) (243 mg, 0.63 mmol), methyl 3-(2-hydroxyphenyl)propanoate (148 mg, 0.819 mmol) and Ph₃P (215 mg, 0.819 mmol) in THF (2 ml) was added a solution of DEAD (143 mg, 0.819 mmol) in THF (1.5 ml) slowly at room temperature. After addition was completed the reaction mixture was stirred at room temperature for 90 min. The volatiles were removed in vacuo and the residue was subjected to flash chromatography (0-1% methanol in dichloromethane containing 0.2% NH₄OH) to give the corresponding N-Boc protected ester derivative, which was dissolved in THF (3 ml). Aqueous NaOH (330 mg NaOH in 1.5 ml of H₂O) was added and the reaction mixture was then stirred at 75° C. for 2 h, cooled to 0° C. and the pH then adjusted to 2 by addition of aqueous 50% TFA. Additional TFA (6 ml) was added and the reaction mixture was stirred at room temperature for 3 h. The volatiles were removed in vacuo and the residue was purified by reverse phase HPLC (10-75% CH₃CN in H₂O containing 0.1% CF₃CO₂H) to give the titled compound (210 mg, 51%).

¹H-NMR (CD₃OD, 400 MHz): δ 7.50 (s, 4H); 7.22-7.15 (m, 2H); 6.93 (d, J=7.9 Hz, 1H); 6.89 (t, J=7.44 Hz, 1H); 4.34 (s, 2H); 4.14 (t, J=5.6 Hz, 2H); 3.62 (d, J=12.9 Hz, 2H); 3.555-3.45 (m, 1H); 3.34 (t, J=7.7 Hz, 2H); 3.11 (t, J=12.9 Hz, 2H); 2.81 (t, J=7.8 Hz, 2H); 2.56 (t, J=8.2 Hz, 2H); 2.40 (d, J=13.3 Hz, 2H); 2.26-2.18 (m, 2H); 2.06-1.92 (m, 2H).

APCI-MS: m/z 431 (MH⁺).

### tert-Butyl [1-(4-chlorobenzyl)piperidin-4-yl](3-hydroxypropyl)carbamate (7a)

To a solution of 3-{[1-(4-chlorobenzyl)piperidin-4-yl]amino}propan-1-ol (7b) (1.50 g, 5.3 mmol) in dichloromethane (20 ml) was added Et₃N (1.48 ml, 10.6 mmol) followed by slow addition of di-tert-butyl dicarbonate (1.16 g, 5.3 mmol) at room temperature and the reaction mixture was stirred at room temperature for 48 h. The volatiles were removed in vacuo and the residue was purified by silica gel flash chromatography (0-2.5% methanol in dichloromethane containing 0.2% NH₄OH) to give the subtitled compound 7a (1.99 g, 98%).

¹H-NMR (CD₃OD, 400 MHz): δ 7.36 (s, 4H); 3.80 (br. S, 1H); 3.53 (t, J=6.3 Hz, 2H); 3.50 (s, 2H); 3.20 (br, s, 2H); 2.92 (d, J=11.7 Hz, 2H); 2.06 (t, J=11.5 Hz, 2H); 1.88-1.70 (m, 4H); 1.64 (d, J=12.2 Hz, 2H); 1.43 (s, 9H).

APCI-MS: m/z 383 (MH⁺) and 283 (M-Boc).

### 3-{[1-(4-Chlorobenzyl)piperidin-4-yl]amino}propan-1-ol (7b)

To a solution of 1-(4-chlorobenzyl)piperidin-4-amine (2.24 g, 10 mmol) in DMF (6 ml) was added Et₃N (4.18 ml, 30 mmol) followed by 3-bromopropan-1-ol (0.9 ml, 10 mmol). After addition was completed the reaction mixture was stirred at room temperature for 36 h. The reaction mixture was partitioned between ethyl acetate and H₂O. The organic layer was dried over Na₂SO₄, filtered and concentrated. The residue was purified by flash chromatography (0-15% methanol in dichloromethane containing 0.2% NH₄OH) to give the subtitle compound (7b) (1.52 g, 54%).

¹H-NMR (CD₃OD, 300 MHz): δ 7.38 (s, 4H); 3.67 (br. t, 2H); 3.50 (s, 2H); 2.88 (br. d, J=11.9 Hz, 2H); 2.73 (t, J=7.3 Hz, 2H); 2.58 (m, 1H); 2.01 (t, J=11.9 Hz, 2H); 1.90 (d, J=12.5 Hz, 2H); 1.70 (m, 2H); 1.50-1.34 (m, 2H).

APCI-MS: m/z 283 (MH⁺).

### EXAMPLE 8

### 3-{2-[((2S)-3-{[1-(4-chlorobenzyl)piperidin-4-yl]amino}-2-hydroxy-2-methylpropyl)oxy]phenyl}propanoic acid bis(trifluoroacetate) (salt)

To a solution of methyl 3-{2-[((2S)-3-{[1-(4-chlorobenzyl)piperidin-4-yl]amino}-2-hydroxy-2-methylpropyl)oxy]phenyl}propanoate (8a) (96 mg, 0.202 mmol) in THF (3 ml) was added aqueous NaOH (330 mg NaOH in 1.5 ml of H₂O) and the reaction mixture was stirred at 75° C. for 2.5 h, cooled to 0° C. and the pH was adjusted to 2 by addition of aqueous 50% TFA. The volatiles were removed in vacuo and the residue was purified by reverse phase HPLC (10-75% CH₃CN in H₂O containing 0.1% TFA) to give the titled compound (115 mg, 83%).

¹H-NMR (CDCl₃, 400 MHz): δ 7.52 (s, 4H); 7.22-7.16 (m, 2H); 6.96-6.89 (m, 2H); 4.30 (s, 2H); 3.98 (d, J=9.5 Hz, 1H); 3.93 (d, J=9.5 Hz, 1H); 3.59 (br. d, J=13.4 Hz, 2H); 3.48 (m, 1H); 3.43 (d, J=13.0 Hz, 1H); 3.23 (d, J=12.6 Hz, 1H); 3.08 (t, J=12.1 Hz, 2H); 2.84 (t, J=7.9 Hz, 2H); 2.65-2.51 (m, 2H); 2.42 (d, J=13.2 Hz, 2H); 2.12 (m, 2H); 1.49 (s, 3H).

APCI-MS: m/z 461 (MH⁺).

### Methyl 3-{2-[((2S)-3-{[1-(4-chlorobenzyl)piperidin-4-yl]amino}-2-hydroxy-2-methylpropyl)oxy]phenyl}propanoate (8a)

Compound I-(4-chlorobenzyl)piperidin-4-amine (8b) (70 mg, 0.31 mmol) and methyl 3-(2-{[(2S)-2-methyloxiran-2-yl]methoxy}phenyl)propanoate (78 mg, 0.31 mmol) in methanol (3 ml) was stirred at 75° C. overnight. The volatiles were removed in vacuo and the residue was purified by flash chromatgraphy (0-2% methanol in dichloromethane containing 0.2% NH₄OH) to give the subtitled compound 8a (100 mg, 70%).

¹H-NMR (CDCl₃, 400 MHz): δ 7.32-7.12 (m, 6H); 6.91 (t, J=7.33 Hz, 1H); 6.85 (d, J=8.7 Hz, 1H); 3.81 (m, 2H); 3.68 (s, 3H); 3.45 (s, 2H); 3.03-2.91 (m, 3H); 2.80 (d, J=11.4 Hz, 2H); 2.66-2.53 (m, 3H); 2.49-2.37 (m, 1H); 2.06-1.80 (m, 4H); 1.40-1.33 (m, 2H); 1.28 (s, 3H).

APCI-MS: m/z 475 (MH⁺).

### Methyl 3-(2-{[(2S)-2-methyloxiran-2-yl]methoxy}phenyl)pronanoate (8b)

A mixture of [(2S)-2-methyloxiran-2-yl]methyl 3-nitrobenzenesulfonate (273 mg, 1 mmol), methyl 3-(2-hydroxyphenyl)propanoate (180 mg, 1 mmol) and Cs₂CO₃ (391 mg, 1.2 mmol) in DMF (5 ml) was stirred at room temperature overnight. The reaction mixture was partitioned between ethyl acetate and H₂O. The organic layer was dried over Na₂SO₄, filtered and the filtrate was concentrated in vacuo. The residue was purified by silica gel flash chromatography (0-20% ethyl acetate in petroleum spirit 40-60° C.) to give subtitled compound (200 mg, 80%).

¹H-NMR (CDCl₃, 400 MHz): δ 7.18 (m, 2H); 6.90 (t, J=7.3 Hz, 1H); 6.80 (d, J=8.0 Hz, 1H); 4.06 (d, J=10.5 Hz, 1H); 3.96 (d, J=10.5 Hz, 1H); 3.68 (s, 3H); 2.98 (t, J=7.8 Hz, 2H); 2.89 (d, J=4.8 Hz, 1H); 2.75 (d, J=4.8 Hz, 1H); 2.64 (t, J=7.8 Hz, 2H) 1.50 (s, 3H).

### EXAMPLE 9

### {5-Chloro-2-[((2S)-3-{[1-(4-chlorobenzyl)piperidin-4-yl]amino}-2-hydroxypropyl)oxy]phenyl}acetic acid bis(trifluoroacetate) (salt)

The titled compound was prepared according to the procedure described in Example 1 from 1-(4-chlorobenzyl)piperidin-4-amine (88 mg, 390 μmol) and methyl {5-chloro-2-[(2S)-oxiran-2-ylmethoxy]phenyl}acetate (9a) (100 mg, 390 μmol). Yield: 148 mg (55%) as a colourless powder.

¹H NMR (400 MHz, CD₃OD) δ 7.51 (m, 4H), 7.26-7.24 (m, 2H), 6.95 (m, 1H), 4.33 (s, 2H), 4.25 (m, 1H), 4.10 (dd, J=9.8, 4.3 Hz, 1H), 4.00 (dd, J=9.8, 6.1 Hz, 1H), 3.61 (m, 4H), 3.50 (m, 1H), 3.36 (dd, J=12.6, 2.9 Hz, 1H), 3.22 (dd, J=12.6, 9.7 Hz, 1H), 3.11 (t, J=12.4 Hz, 2H), 2.42 (t, J=13.5 Hz, 2H), 2.01 (m, 2H)

APCI-MS m/z: 467.1, 469.1 [MH⁺].

### Methyl {5-chloro-2-[(2S)-oxiran-2-ylmethoxy]phenyl}acetate (9a)

The subtitled compound was prepared according to the protocol described for 1a starting from methyl (5-chloro-2-hydroxyphenyl)acetate (5b, 200 mg, 1.00 mmol) and (2S)-oxiran-2-ylmethyl 3-nitrobenzenesulfonate (259 mg, 1.00 mmol). Yield: 200 mg (78%) as brown oil.

¹H NMR (400 MHz, CDCl₃) δ 7.22 (d, J=2.5 Hz, 1H), 7.19 (dd, J=3.9, 2.5 Hz, 1H), 6.80 (d, J=8.5 Hz, 1H), 4.25 (dd, J=11.1, 2.8 Hz, 1H), 3.95 (dd, J=11.1, 5.4 Hz, 1H), 3.72 (s, 3H), 3.63 (m, 2H), 3.32 (dquintet, J=4.1, 2.7 Hz, 1H), 2.89 (t, J=4.5 Hz, 1H), 2.76 (dd, J=4.9, 2.6 Hz, 1H).

APCI-MS m/z: 298.1, 300.1 [MH⁺+CH₃CN].

### EXAMPLE 10

### [2-(3-{[1-(4-Chlorobenzyl)piperidin-4-yl]amino}propoxy)phenyl]acetic acid bis (trifluoroacetate) (salt)

To a solution of tert-butyl [1-(4-chlorobenzyl)piperidin-4-yl](3-hydroxypropyl)carbamate (7a) (192 mg, 0.5 mmol), Ph₃P (157 mg, 0.6 mmol) and methyl (2-hydroxyphenyl)acetate (83 mg, 0.5 mmol) in THF (2 ml) was added a solution of DEAD (105 mg, 0.6 mmol) in THF (1.5 ml) slowly at room temperature. After addition was completed the reaction mixture was stirred at room temperature overnight. The volatiles were removed in vacuo and the residue was purified by silica gel flash chromatography (0-1.5% methanol in dichloromethane containing 0.2% NH₄OH) to give the corresponding N-Boc protected ester derivative containing impurities. The residue was dissolved in THF (3 ml) and aqueous NaOH (360 mg NaOH in 1.5 ml H₂O) was added. The reaction mixture was stirred at 78° C. for 4 h, cooled to 0° C. and the pH adjusted to 2 by addition of aqueous 50% TFA. An additional 6 ml of TFA was added and the reaction mixture stirred at room temperature over night. The volatiles were removed in vacuo and the residue was purified by reverse phase HPLC (10-70% CH₃CN in H₂O containing 0.1% TFA) to give the titled compound (146 mg, 35%).

¹H-NMR (CDCl₃, 400 MHz): δ 7.50 (s, 4H); 7.26 (m, 1H); 7.20 (d, J=7.4 Hz, 1H); 6.99-6.90 (m, 2H); 4.33 (s, 2H); 4.14 (t, J=5.5 Hz, 2H); 3.60 (m, 4H); 3.45 (m, 1H); 3.28 (m, 2H); 3.09 (t, J=12.7 Hz, 2H); 2.39 (d, J=13.7 Hz, 2H); 2.18 (m, 2H); 1.98 (m, 2H).

APCI-MS: m/z 417 (MH⁺).

### EXAMPLE 11

### 3-{5-Chloro-2-[((2S)-3-{[1-(4-chlorobenzyl)piperidin-4-yl]amino}-2-hydroxy-2-methylpropyl)oxy]phenyl}propanoic acid bis(trifluoroacetate) (salt)

Methyl 3-{5-chloro-2-[((2S)-3-{[1-(4-chlorobenzyl)piperidin-4-yl]amino}-2-hydroxy-2-methylpropyl)oxy]phenyl}propanoate (11a) (95 mg, 0.186 mmol) was dissolved in THF (3 ml), aqueous NaOH (165 mg NaOH in 1.5 ml H₂O) was added and the reaction mixture stirred at 78° C. for 3 h, cooled to 0° C. and pH adjusted to 2 by addition of aqueous CF₃CO₂H. The volatiles were removed in vacuo and the residue was purified by reverse phase HPLC (10-75% CH₃CN in H₂O containing 0.1% TFA) to give the titled compound (105 mg, 75%).

¹H-NMR (CD₃OD, 400 MHz): δ 7.50 (m, 4H); 7.22-7.17 (m, 2H); 6.92 (d, J=8.5 Hz, 1H); 4.30 (s, 2H); 3.97 (d, J=9.5 Hz, 1H); 3.92 (d, J=9.5 Hz, 1H); 3.58 (d, J=12.8 Hz, 2H); 3.46 (m, 1H); 3.40 (d, J=12.7 Hz, 1H); 3.20 (d, J=12.7 Hz, 1H); 3.04 (t, J=12.8 Hz, 2H); 2.92 (t, J=7.6 Hz, 2H); 2.58 (m 2H); 2.40 br. d, 2H); 2.00 (m, 2H); 1.43 (s, 3H).

APCI-MS: m/z 495 (MH⁺).

### Methyl 3-{5-chloro-2-[((2S)-3-{[1-(4-chlorobenzyl)piperidin-4-yl]amino}-2-hydroxy-2-methylpropyl)oxy]phenyl}propanoate (11a)

A suspension of [(2S)-2-methyloxiran-2-yl]methyl 3-nitrobenzenesulfonate (95 mg, 0.349 mmol), methyl 3-(5-chloro-2-hydroxyphenyl)propanoate (75 mg, 0.349 mmol) and Cs₂CO₃ (136 mg, 0.419 mmol) in DMF (2 ml) was stirred at room temperature overnight. The reaction mixture was partitioned between ethyl acetate and H₂O. The organic layer was dried over Na₂SO₄, filtered and concentrated in vacuo. The residue was dissolved in methanol (3 ml), 1-(4-chlorobenzyl)piperidin-4-amine (78 mg, 0.349 mmol) was added and the resulting solution stirred at 78° C. overnight. The volatiles were removed in vacuo and the resulting residue was purified by silica gel flash chromatography (0-2% methanol in dichloromethane containing 0.2% NH₄OH) to give the subtitled compound (11a) (100 mg, 56%). ¹H-NMR (CD₃OD, 400 MHz): δ 7.34-7.26 (m, 4H); 7.18-7.13 (m, 2H); 6.90 (m, 1H); 3.91 (d, J=9.0 Hz, 1H); 3.80 (d, J=9.0 Hz, 1H); 3.62 (s, 3H); 3.38 (s, 2H); 2.95-2.77 (m, 5H); 2.71 (d, J=11.9 Hz, 1H); 2.60 (t, J=7.7 Hz, 2H); 2.48 (m, 1H); 2.06 (m, 2H); 1.88 (m, 2H); 1.40 (m, 2H); 1.30 (s, 3H).

APCI-MS: m/z 509 (MH⁺).

### EXAMPLE 12

### [5-Chloro-2-(3-{[1-(4-chlorobenzyl)piperidin-4-yl]amino}propoxy)phenyl]acetic acid bis(trifluoroacetate) (salt)

The titled compound was prepared according to the protocol described in Example 7, starting from tert-butyl[1-(4-chlorobenzyl)piperidin-4-yl](3-hydroxypropyl)carbamate (7a, 153 mg, 400 μmol) and methyl (5-chloro-2-hydroxyphenyl)acetate (80 mg, 400 μmol).

Yield: 131 mg (48%).

¹H NMR (400 MHz, CD₃OD) δ 7.52-7.45 (m, 4H), 7.27-7.21 (m, 2H), 6.95 (d, J=9.2 Hz, 1H), 4.23 (s, 2H), 4.13 (t, J=5.5 Hz, 2H), 3.59 (m, 2H), 3.53 (dd, J=15.6, 8.1 Hz, 2H), 3.41 (m, 1H), 3.27 (t, J=7.6 Hz, 2H), 2.99 (t, J=12.4 Hz, 2H), 2.35 (d, J=13.4 Hz, 2H), 2.18 (m, 2H), 1.94 (m, 2H).

### EXAMPLE 13

### 3-{2-[((2S)-3-{[1-(4-Chlorobenzyl)piperidin-4-yl]amino}-2-hydroxy-2-methylpropyl)oxy]-5-fluorophenyl}propanoic acid bis(trifluoroacetate) (salt)

Methyl 3-{2-[((2S)-3-{[1-(4-chlorobenzyl)piperidin-4-yl]amino}-2-hydroxy-2-methylpropyl)oxy]-5-fluorophenyl}propanoate (13a) (68 mg, 0.094 mmol) was dissolved in THF (3 ml), aqueous NaOH (165 mg NaOH in 1.5 ml H₂O) was added and the reaction mixture was stirred at 80° C. for 3 h, cooled to 0° C. and the pH adjusted to 2 by addition of aqueous 50% TFA. The volatiles were removed in vacuo and the residue was purified by reverse phase HPLC (10-75% CH₃CN in H₂O containing 0.1% CF₃CO₂H) to give the titled compound (47 mg, 71%).

¹H-NMR. (CD₃OD, 400 MHz): δ 7.52 (m 4H); 6.98-6.90 (m, 3H); 4.38 (s, 2H); 3.95 (d, J=9.4 Hz, 1H); 3.91 (d, J=9.4 Hz, 1H); 3.61 (br.d, J=12.9 Hz, 2H); 3.50 (m, 1H); 3.41 (d, J=12.8 Hz, 1H); 3.22 (d, J=12.8 Hz, 1H); 3.12 (m, 2H); 2.93 (t, J=5.9 Hz, 2H); 2.66-2.52 (m, 2H); 2.42 (m, 2H); 2.01 (m, 2H); 1.44 (s, 31-1).

APCI-MS: m/z 479 (MH⁺).

### Methyl 3-{2-[((2S)-3-{[1-(4-chlorobenzyl)piperidin-4-yl]amino}-2-hydroxy-2-methylpropyl)oxy]-5-fluorophenyl}propanoate (13a)

Methyl (2E)-3-{2-[((2S)-3-{[1-(4-chlorobenzyl)piperidin-4-yl]amino}-2-hydroxy-2-methylpropyl)oxy]-5-fluorophenyl}acrylate (150 mg, 0.305 mmol) was taken into ethyl acetate (9 ml), Pt/C (5%, 45 mg) was added and the solution hydrogeneted at atmospheric pressure and at room temperature for 24 h. The catalyst was filtered off and the filtrate was concentrated in vacuo to give the subtitled compound 13a (83 mg, 38%). ¹H-NMR (CD₃OD, 400 MHz): δ 7.51 (s, 4H); 6.97-6.89 (m, 3H); 4.38 (s, 2H); 3.95 (d, J=9.4, 1H); 3.91 (d, J=9.4 Hz, 1H); 3.68-3.60 (m, 5H); 3.50 (m, 1H); 3.41 (d, J=12.7 Hz, 1H); 3.22 (d, J=12.7 Hz, 1H); 3.12 (t, J=12.6 Hz, 2H); 2.94 (t, J=7.7 Hz, 2H); 2.62 (m, 2H); 2.44 (m, 2H); 2.01 (m, 2H); 1.45 (s, 3H).

APCI-MS: m/z 493 (MH⁺).

### Methyl (2E)-3-{2-[((2S)-3-{[1-(4-chlorobenzyl)piperidin-4-yl]amino}-2-hydroxy-2-methylpropyl)oxy]-5-fluorophenyl}acrylate (13b)

1-(4-Chlorobenzyl)piperidin-4-amine (126 mg, 0.563 mmol) and methyl(2E)-3-(5-fluoro-2-{[(2S)-2-methyloxiran-2-yl]methoxy}phenyl)acrylate (13c) (150 mg, 0.563 mmol) in methanol (3 ml) was stirred at 80° C. over night. The volatiles were removed in vacuo and the residue was purified by flash chromatography (0-2% methanol in dichloromethane containing 0.2% NH₄OH) to give the subtitled compound (13b) (220 mg, 80%).

¹H-NMR (CD₃OD, 400 MHz): δ 7.93 (dd, J=1.0, 16.1 Hz, 1H); 7.32-7.20 (m, 5H); 7.07-7.00 (m, 1H); 6.89 (dd, J=4.4, 9.1 Hz, 1H); 6.47 (d, J=16.1 Hz, 1H); 3.84 (s, 2H); 3.79 (s, 3H); 3.44 (s, 2H); 3.00 (d, J=12.5 Hz, 1H); 2.79 (m, 2H); 2.58 (d, 12.5 Hz, 1H); 2.42 (m, 1H); 1.99 (m, 2H); 1.84 (m, 2H); 1.34 (m, 2H); 1.28 (s, 3H).

APCI-MS: m/z 491 (MH⁺).

### Methyl (2E)-3-(5-fluoro-2-{[(2S)-2-methyloxiran-2yl]methoxy}phenyl)acrylate (13c)

A mixture of methyl (2E)-3-(5-fluoro-2-hydroxyphenyl)acrylate (13d) (196 mg, 1 mmol), [(2S)-2-methyloxiran-2-yl]methyl 3-nitrobenzenesulfonate (273 mg, 1 mmol) and Cs₂CO₃ (391 mg, 1.2 mmol) in DMF (5 ml) was stirred at room temperature overnight. The reaction mixture was partitioned between ethyl acetate and H₂O. The organic layer was dried over Na₂SO₄, filtered and concentrated in vacuo. The residue was purified by silica gel flash chromatography (0-20% ethyl acetate in petroleum spirit 40-60° C.) to give the subtitled compound 13c (215 mg, 81%).

¹H-NMR (CDCl₃, 400 MHz): δ 7.98 (dd, J=1.0, 16.1 Hz, 1H); 7.23 (dd, J=3.1, 9.1 Hz, 1H); 7.07-7.00 (m, 1H); 6.88 (dd, J=4.4, 9.1 Hz, 1H); 6.48 (d, J=16.1 Hz, 1H); 4.10 (d, J=10.6 Hz, 1H); 3.98 (d, J=10.6 Hz, 1H); 3.83 (s, 3H); 2.88 (d, J=4.6 Hz, 1H); 2.78 (d, J=4.6 Hz, 1H); 1.52 (s, 3H).

### Methyl (2E)-3-(5-fluoro-2-hydroxyphenyl)acrylate^{l} (13d)

To a solution of 5-fluoro-2-hydroxybenzaldehyde (280 mg, 2 mmol) in THF (6 ml) was added a solution of methyl (triphenylphosphoranylidene)acetate (669 mg, 2 mmol) in dichloromethane (4 ml) dropwise at 3° C. After addition was completed, the ice-bath was removed and the reaction mixture was stirred at room temperature for 24 h. The volatiles were removed in vacuo and the residue was purified by flash chromatography (0-20% ethyl acetate in petroleum ether 40-60° C.) to give the subtitled compound 13d (335 mg, 85%). (Anwar, H. F; Skattebol, L; Skramstad, J; Hansen, T. V. Tetrahedron Lett. 2005, 46, 5285-5287)

¹H-NMR (DMSO-d₆, 400 MHz): δ 10.20 (br.s, 1H); 7.83 (dd, J=1.7, 16.1 Hz, 1H); 7.51 (dd, J=3.1, 9.7 Hz, 1H); 7.13-7.04 (m, 1H); 6.89 (dd, J=4.9, 9.0 Hz, 1H); 6.67 (d, J=16.1 Hz, 1H); 3.68 (s, 3H).

### EXAMPLE 14

### (2-{[(2S)-3-{[1-(4-chlorobenzyl)piperidin-4-yl]amino}-2-hydroxypropyl]oxy}-4-fluorophenyl)acetic acid bis(trifluoroacetate) (salt)

The title compound was prepared according to the procedure described in Example 1 starting from 1-(4-chlorobenzyl)piperidin-4-amine (49 mg, 217 μmol) and crude methyl {4-fluoro-2-[(2S)-oxiran-2-ylmethoxy]phenyl}acetate (14a, 98 mg, approx. 200 μmol). 36 mg (24%, two steps) of the title compound were obtained as a colourless powder.

¹H NMR (300 MHz, DMSO-d₆): δ 12.49 (s, 1H), 9.78 (s, 1H), 8.83 (s, 2H), 7.55 (d, J=8.6 Hz, 4H), 7.23 (dd, J=8.3, 7.0 Hz, 1H), 6.91 (dd, J=11.2, 2.4 Hz, 1H), 6.75 (td, J=8.4, 2.4 Hz, 1H), 5.89 (s, 1H), 4.31 (s, 2H), 4.13 (s, 1H), 4.08-3.92 (m, 2H), 3.55-2.84 (m, 9H), 2.32-2.12 (m, 2H), 1.75 (m, 2H).

LC (method A) rt=5.76 min. 97% LTV 220 nm.

LC (method B) rt=4.63 min.>99% UV 220 nm.

### Methyl {4-fluoro-2-[(2S)-oxiran-2-ylmethoxy]phenyl}acetate (14a)

The subtitle compound 14a was prepared according to the procedure described in example 1a starting from methyl (4-fluoro-2-hydroxyphenyl)acetate (1b, 40 mg, 217 μmol) and [(2S)-oxiran-2-yl]methyl 3-nitrobenzenesulfonate (56 mg, 217 μmol). 98 mg of the crude was obtained and used in the next step without further purification or analysis.

### EXAMPLE 15

### 3-[2-(3-{[1-(4-Chlorobenzyl)piperidin-4-yl]amino}propoxy)-4-fluorophenyl]propionic acid bis(trifluoroacetate) (salt)

To a solution of tert-butyl [1-(4-chlorobenzyl)piperidin-4-yl](3-hydroxypropyl)carbamate (Example 7) (154 mg, 0.4 mmol), methyl 3-(4-fluoro-2-hydroxyphenyl)propanoate (prepared in Example 6) (80 mg, 0.4 mmol) and Ph₃P (126 mg, 0.48 mmol) in THF (1 ml) was added a solution of DEAD (84 mg, 0.48 mmol) in THF (1 ml) slowly at room temperature. After addition was completed the reaction mixture was stirred at room temperature over night. The volatiles were removed in vacuo and the residue was dissolved in THF (3 ml) and aqueous NaOH (165 mg NaOH in 1.5 ml water) was added. The reaction mixture was stirred at 80° C. for 6 h, cooled to 0° C. and pH was adjusted to 2 by addition of aquous TFA, additional TFA (6 ml) was added and the reaction mixture was stirred at room temperature over night. The volatiles were removed in vacuo and the residue was purified by HPLC (10-75% acetonitrile in water, 0.1% TFA) to give title compound (42 mg).

¹H-NMR (CD₃OD, 400 MHz): δ 7.50 (m, 4H); 7.16 (dd, J=6.7, 8.3 Hz, 1H); 6.74 (dd, J=2.5, 11.0 Hz, 1H); 6.62 (m, 1H); 4.28 (s, 2H); 4.12 (t, J=5.7 Hz, 2H); 3.56 (br.d, J=12.9 Hz, 2H); 3.45 (m, 1H); 3.33 (m, under MeOH peak, 2H); 3.03 (t, J=12.0 Hz, 2H); 2.87 (t, J=7.6 Hz, 2H); 2.53 (t, J=7.8 Hz, 2H); 2.37 (br.d, J=3.5 Hz, 2H); 2.22 (m, 2H); 1.95 (m, 2H).

APCI-MS: m/z 449 (MH⁺).

### EXAMPLE 16

### [2-{[(2S)-3-{[1-(4-Chlorobenzyl)piperidin-4-yl]amino}-2-hydroxy-2-methylpropyl]oxy}-4-(trifluoromethyl)phenyl]acetic acid bis(trifluoroacetate) (salt)

The title compound was prepared according to the procedure described in Example 1 starting from 1-(4-chlorobenzyl)piperidin-4-amine (45 mg, 200 μmol) and methyl {4-trifluoromethyl-2-[(2S)-2-methyloxiran-2-ylmethoxy]phenyl}acetate (16a, 61 mg, 200 μmol). Yield: 48 mg (33%) as a colourless powder.

¹H NMR (400 MHz, CD₃OD): δ 7.50 (m, 4H), 7.43 (d, J=7.8 Hz, 1H), 7.28 (d, J=8.0 Hz, 1H), 7.22 (s, 1H), 4.32 (s, 2H), 4.02 (dd, J=21.7, 9.5 Hz, 2H), 3.71 (m, 2H), 3.61 (d, J=12.2 Hz, 2H), 3.47 (m, 1H), 3.39 (d, J=12.7 Hz, 1H), 3.17 (d, J=12.7 Hz, 2H), 3.09 (t, J=13.0 Hz, 2H), 2.43 (d, J=13.3 Hz, 2H), 2.03 (m, 2H), 1.45 (s, 3H).

APCI-MS m/z: 515 [MH⁺].

### Methyl (4-trifluoromethyl-2-{[12S)-2-methyloxiran-2-yl]methoxy}phenyl)acetate (16a)

The subtitled compound was prepared according to the protocol described for 1a starting from methyl (4-trifluoromethyl-2-hydroxyphenyl)acetate (16b, 89 mg, 380 μmol) and (2S)-2-methyloxiran-2-ylmethyl 3-nitrobenzenesulfonate (103 mg, 380 μmol). Yield: 103 mg (89%) as red oil.

¹H NMR (400 MHz, CDCl₃): δ 7.31 (d, J=7.8 Hz, 2H), 7.22 (d, J=7.8 Hz, 2H), 7.06 (s, 1H), 4.12 (d, J=10.3 Hz, 1H), 3.96 (d, J=10.3 Hz, 1H), 3.71 (s, 3H), 2.96 (s, 2H), 2.88 (d, J=4.8 Hz, 1H), 2.75 (d, J=4.6 Hz, 1H), 1.48 (s, 3H).

### Methyl [2-hydroxy-4-(trifluoromethyl)phenyl]acetate (16b)

The subtitled compound was prepared according to the protocol described for 1b from [2-hydroxy-4-(trifluoromethyl)benzyl](triphenyl)phosphonium bromide (16c, 425 mg, 1.24 mmol) and methyl chloroformate (117 mg, 1.24 mmol). 89 mg (31%) of 16b was obtained as red oil.

### [2-hydroxy-4-(trifluoromethyl)benzyl](triphenyl)phosphonium bromide (16c)

The subtitled compound 16c was prepared according to the protocol described by J. Guillaumel et al., Eur. J. Med. Chem. 1983, 18 (5), 431-436, 2-(hydroxymethyl)-5-(trifluoromethyl)phenol (16d, 239 mg, 1.24 mmol) and triphenylphosphine hydrobromide (425 mg, 1.24 mmol). 645 mg (quanitative) of 16c was obtained as brown oil.

### 2-(Hydroxymethyl)-5-(trifluoromethyl)phenol (16d)

The subtitled compound was prepared according to the protocol described for 1d from methyl 2-hydroxy-4-(trifluoromethyl)benzoate (300 mg, 1.36 mmol). 239 mg (91%) of 16d was obtained.

### EXAMPLE 17

### (2-{[(2S)-3-{[1-(4-fluorobenzyl)piperidin-4-yl]amino}-2-hydroxypropyl]oxy}phenyl)acetic acid bis(trifluoroacetate) (salt)

LiClO₄ (95 mg, 0.9 mmol) and a solution of methyl {2-[(2S)-2-methyloxiran-2-ylmethoxy]phenyl}acetate (17a, 33 mg, 0.15 mmol) in CH₃CN (1 ml) were added to a solution of 1-(4-fluorobenzyl)piperidin-4-amine (17b, 46 mg, 0.22 mmol) in EtOH and the mixture was stirred at 75° C. for 3 h. The resulting mixture was concentrated to remove the EtOH and then diluted with CH₃CN (1.0 ml). An aqueous solution of LiOH (1.0 ml, 1.0 mmol) was added and the reaction mixture was stirred at room temperature for 16 h and then acidified by addition of AcOH. The crude product was purified by reversed phase HPLC (Kromasil column C₁₈, 100×30 mm, 5-50% CH₃CN in H₂O containing 0.1% CF₃CO₂H) to give the title compound as a white solid in 12 mg (11%) after freeze-drying.

¹H NMR (CD₃OD, 400 MHz): δ 7.53 (m, 2H), 7.24 (m, 4H), 6.96 (m, 2H), 4.25 (br s, 3H), 4.13 (dd, J=10.0, 4.3 Hz, 1H), 4.00 (dd, J=10.1, 6.6 Hz, 1H), 3.62 (d, J=4.6 Hz, 2H), 3.60-3.52 (m, 2H), 3.51-3.41 (m, 1H), 3.38 (dd, J=12.6, 3.1 Hz, 1H), 3.23 (dd, J=12.6, 9.1 Hz, 1H), 3.07-2.94 (m, 2H), 2.45-2.35 (m, 2H), 2.05-1.89 (m, 2H).

APCI-MS: m/z 417 [MH⁺].

LC (method A) R_{T}=4.88 min. 93.5% UV 220 nm.

LC (method B) R_{T}=3.52 min. 97.7% UV 220 nm.

### {2-[(2S)-2-methyloxiran-2-ylmethoxy]phenyl}acetate(17a)

The subtitle compound was prepared according to the protocol described for 1a starting from methyl 2-(2-hydroxyphenyl)acetate (3.14 g, 12.1 mmol) and (2S)-oxiran-2-ylmethyl 3-nitrobenzenesulfonate (1.83 g, 11.0 mmol). The crude subtitle compound was obtained in 2.617 g (107%) as oil.

APCI-MS: m/z 264 [MH⁺+CH₃CN].

### 1-(4-Fluorobenzyl)piperidin-4-amine (17b)

4-Fluorobenzyl chloride (433 mg, 3 mmol) was dissolved in CH₂Cl₂ (7 ml) and added to tert-butyl piperidin-4-ylcarbamate (400 mg, 2.0 mmol) at room temperature. DIEA (525 3.0 mmol) was added and the resulting mixture was stirred for 64 h. TFA (2 ml) was added and the mixture was stirred at room temperature for 1 h. The volatiles were then removed in vacuo and the resulting residue dissolved in AcOH (40 ml, 2 M in EtOH). Purification with ion exchange resin (Varian Bond Elut SCX 10g, washing with EtOH and eluating with NH₃ (8% in EtOH)) gave the subtitle compound 17a in 345 mg (83%).

APCI-MS: m/z 209 [MH⁺].

The following 1-arylpiperidin-4-amines were prepared according to the procedure described for 17b.

| | APCI-MS, | |
|---|---|---|
| 1-Arylpiperidin-4-amines | m/z ([MH⁺]) | Compound |
| 1-(4-chloro-3- | 293 | 18a |
| (trifluoromethyl)benzyl)piperidin-4-amine | | |
| 1-(3,4-difluorobenzyl)piperidin-4-amine | 227 | 19a |
| 1-(2,5-dimethylbenzyl)piperidin-4-amine | 219 | 20a |
| 1-(2-chloro-5-fluorobenzyl)piperidin-4-amine | 243 | 21a |
| 1-(3-chloro-4-fluorobenzyl)piperidin-4-amine | 243 | 22a |
| 1-(3,5-dimethylbenzyl)piperidin-4-amine | 219 | 23a |
| 1-(4-chloro-2-fluorobenzyl)piperidin-4-amine | 243 | 24a |
| 1-(4-bromobenzyl)piperidin-4-amine | 269, 271 | 25a |

### EXAMPLE 18

### (2-{[(2S)-3-({1-[4-Chloro-3-(trifluoromethyl)benzyl]piperidin-4-yl}amino)-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(4-chloro-3-(trifluoromethyl)benzyl)piperidin-4-amine (18a, 64 mg, 0.22 mmol) and obtained as a solid in 22 mg (20%).

¹H NMR (400 MHz, CD₃OD): δ 7.97 (s, 1H), 7.75 (s, 2H), 7.26 (t, J=7.6 Hz, 1H), 7.22 (d, J=7.6 Hz, 1H), 6.95 (m, 2H), 4.37 (s, 2H), 4.25 (m, 1H), 4.13 (dd, J=9.8, 4.5 Hz, 1H), 4.00 (dd, J=9.8, 6.0 Hz, 1H), 3.62 (d, J=4.2 Hz, 2H), 3.58 (m, 2H), 3.54-3.44 (m, 1H), 3.38 (dd, J=13.1, 2.8 Hz, 1H), 3.23 (dd, J=12.6, 9.3 Hz, 1H), 3.08 (br t, J=12.6 Hz, 2H), 2.42 (br t, J=13.4 Hz, 2H), 2.08-1.92 (m, 2H).

APCI-MS: m/z 501 [MH⁺].

LC (method A) R_{T}=6.79 min. 97.3% LTV 220 nm.

LC (method B) R_{T}=5.49 min. 97.7% UV 220 nm.

### EXAMPLE 19

### (2-{[(2S)-3-{[1-(3,4-Difluorobenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(3,4-difluorobenzyl)piperidin-4-amine (19a, 50 mg, 0.22 mmol) and obtained as a solid in 3 mg (3%).

¹H-NMR (CD₃OD, 400 MHz): δ 7.47 (br t, J=9.6 Hz, 1H), 7.39 (br q, J=9.0 Hz, 1H), 7.31 (m, 1H), 7.26 (br t, J=8.1 Hz, 1H), 7.22 (br d, J=7.1 Hz, 1H), 6.97 (d, J=8.1 Hz, 1H), 6.94 (t, J=7.2 Hz, 1H), 4.25 (m, 1H), 4.21 (s, 2H), 4.13 (dd, J=9.6, 4.3 Hz, 1H), 4.01 (dd, J=10.1, 6.3 Hz, 1H), 3.62 (d, J=4.8 Hz, 2H), 3.59-3.41 (m, 3H), 3.38 (dd, J=12.4, 2.8 Hz, 1H), 3.23 (dd, J=12.4, 9.1 Hz, 1H), 2.97 (br t, J=12.2 Hz, 2H), 2.39 (br t, J=13.4 Hz, 2H), 2.04-1.88 (m, 2H).

APCI-MS: m/z 435 [MH⁺].

LC (method A) R_{T}=5.28 min. 94.3% UV 220 nm.

LC (method B) R_{T}=4.12 min. 99.2% LTV 220 nm.

### EXAMPLE 20

### (2-{[(2S)-3-{[1-(2,5-Dimethylbenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(2,5-dimethylbenzyl)piperidin-4-amine (20a, 48 mg, 0.22 mmol) and obtained as a solid in 22 mg (20%).

¹H-NMR (CD₃OD, 400 MHz): S 7.29-7.17 (m, 5H), 6.97 (d, J=8.1 Hz, 1H), 6.95 (t, J=7.6 Hz, 1H), 4.30 (s, 2H), 4.26 (m, 1H), 4.13 (dd, J=10.1, 4.9 Hz, 1H), 4.00 (dd, J=10.0, 6.2 Hz, 1H), 3.62 (m, 2H), 3.62 (d, J=4.3 Hz, 2H), 3.55-3.44 (m, 1H), 3.39 (dd, J=12.5, 3.3 Hz, 1H), 3.24 (dd, J=12.5, 9.6 Hz, 1H), 3.20-3.09 (m, 2H), 2.47-2.35 (m, 2H), 2.40 (s, 3H), 2.34 (s, 3H), 2.09-1.91 (m, 2H).

APCI-MS: m/z 427 [MH⁺].

LC (method A) R_{T}=5.66 min. 95.5% UV 220 nm.

LC (method B) R_{T}=4.70 min. 98.2% UV 220 nm.

### EXAMPLE 21

### (2-{[(2S)-3-{[1-(2-Chloro-4-fluorobenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(2-chloro-5-fluorobenzyl)piperidin-4-amine (21a, 53 mg, 0.22 mmol) and obtained as a solid in 17 mg (17%).

¹H-NMR (CD₃OD, 400 MHz): δ 7.68 (dd, J=8.7, 6.0 Hz, 1H), 7.43 (dd, J=8.6, 2.6 Hz, 1H), 7.30-7.19 (m, 3H), 6.95 (m, 2H), 4.40 (s, 2H), 4.25 (m, 1H), 4.13 (dd, J=9.7, 4.6 Hz, 1H), 4.01 (dd, J=10.3, 6.6 Hz, 1H), 3.62 (d, J=4.2 Hz, 2H), 3.60 (m, 2H), 3.53-3.43 (m, 1H), 3.39 (dd, J=12.8, 3.1 Hz, 1H), 3.24 (dd, J=12.8, 9.2 Hz, 1H), 3.13 (br t, J=12.6 Hz, 2H), 2.40 (br t, J=13.1 Hz, 2H), 2.08-1.92 (m, 2H).

APCI-MS: m/z 451 [MH⁺].

LC (method A) R_{T}=5.18 min. 96.5% UV 220 nm.

LC (method B) R_{T}=4.63 min. 99.4% UV 220 nm.

### EXAMPLE 22

### (2-{[(2S)-3-{[1-(3-Chloro-4-fluorobenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(3-chloro-4-fluorobenzyl)piperidin-4-amine (22a, 53 mg, 0.22 mmol) and obtained as a solid in 17 mg (17%).

¹H-NMR (CD₃OD, 400 MHz): δ 7.66 (dd, J=7.2, 2.2 Hz, 1H), 7.45 (m, 1H), 7.35 (t, J=8.9 Hz, 1H), 7.27 (td, J=7.9, 1.8 Hz, 1H), 7.22 (dd, J=7.5, 1.8 Hz, 1H), 6.95 (m, 2H), 4.25 (m, 1H), 4.16 (s, 2H), 4.13 (dd, J=10.1, 4.7 Hz, 1H), 4.00 (dd, J=10.4, 6.4 Hz, 1H), 3.62 (d, J=3.9 Hz, 2H), 3.55-3.40 (m, 3H), 3.37 (dd, J=12.8, 2.8 Hz, 1H), 3.23 (dd, J=12.5, 9.3 Hz, 1H), 2.98-2.84 (m, 2H), 2.37 (br t, J=12.5 Hz, 2H), 1.94 (br t, J=12.3 Hz, 2H).

APCI-MS: m/z 451 [MH⁺].

LC (method A) R_{T}=5.68 min. 99.0% UV 220 nm.

LC (method B) R_{T}=4.51 min. 96.0% UV 220 nm.

### EXAMPLE 23

### (2-{[(2S)-3-{[1-(3,5-Dimethylbenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(3,5-dimethylbenzyl)piperidin-4-amine (23a, 48 mg, 0.22 mmol) and obtained as a solid in 12 mg (12%).

¹H-NMR (CDCl₃, 400 MHz): δ 7.26 (td, J=7.8, 1.8 Hz, 1H), 7.21 (br d, J=17.9 Hz, 1H), 7.18 (s, 1H), 7.13 (s, 2H), 6.95 (m, 1H), 4.25 (m, 1H), 4.25 (s, 2H), 4.13 (dd, J=10.3, 4.4 Hz, 1H), 4.00 (dd, J=10.2, 6.6 Hz, 1H), 3.68-3.56 (m, 3H), 3.55-3.44 (m, 1H), 3.38 (dd, J=12.8, 2.6 Hz, 1H), 3.23 (dd, J=12.4, 9.5 Hz, 1H), 3.17-3.05 (m, 2H), 2.43 (br t, J=13.0 Hz, 2H), 2.35 (s, 6H), 2.09-1.91 (m, 2H).

APCI-MS: m/z 427 [MH⁺].

LC (method A) R_{T}=5.89 min. 97.6% UV 220 nm.

LC (method B) R_{T}=4.71 min. 100% UV 220 nm.

### EXAMPLE 24

### (2-{[(2S)-3-{[1-(4-Chloro-2-fluorobenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(4-chloro-2-fluorobenzyl)piperidin-4-amine (24a, 53 mg, 0.22 mmol) and obtained as a solid in 18 mg (18%).

¹H-NMR (CD₃OD, 400 MHz): δ 7.57 (t, J=8.0 Hz, 1H), 7.42 (dd, J=10.1, 2.4 Hz, 1H), 7.37 (dd, J=8.1, 2.0 Hz, 1H), 7.27 (td, J=7.9, 1.6 Hz, 1H), 7.21 (br d, J=18.3 Hz, 1H), 6.95 (m, 2H), 4.37 (s, 2H), 4.25 (m, 1H), 4.13 (dd, J=9.8, 4.9 Hz, 1H), 4.00 (dd, J=9.3, 6.1 Hz, 1H), 3.65 (m, 4H), 3.48 (m, 1H), 3.38 (dd, J=12.6, 3.6 Hz, 1H), 3.23 (dd, J=12.6, 9.3 Hz, 1H), 3.14 (br t, J=12.6 Hz, 2H), 2.43 (br t, J=13.4 Hz, 2H), 2.01 (br quintet, J=12.8 Hz, 2H).

APCI-MS: m/z 451 [MH⁺].

LC (method A) R_{T}=5.39 min. 99.2% UV 220 nm.

LC (method B) R_{T}=4.58 min. 98.6% UV 220 nm.

### EXAMPLE 25

### (2-{[(2S)-3-{[1-(4-Bromobenzyl)piperidin-4-yl]amino}-2-hydroxypropyl]oxy}phenyl)acetic acid bis(trifluoroacetate) (salt)

The title compound was prepared according to the procedure described in Example 17 starting from {2-[(2S)-2-methyloxiran-2-ylmethoxy]phenyl}acetate (17a, 33 mg, 0.15 mmol) and 1-(4-bromobenzyl)piperidin-4-amine (25a, 59 mg, 0.22 mmol) and obtained as a solid in 29 mg (27%).

¹H-NMR (CD₃OD, 400 MHz): δ 7.66 (d, J=8.3 Hz, 2H), 7.42 (d, J=8.3 Hz, 2H), 7.27 (td, J=7.7, 1.9 Hz, 1H), 7.21 (dd, J=7.6, 1.5 Hz, 1H), 6.95 (m, 2H), 4.25 (m, 1H), 4.23 (s, 2H), 4.13 (dd, J=9.5, 4.1 Hz, 1H), 4.00 (dd, J=9.8, 6.4 Hz, 1H), 3.62 (d, J=4.6 Hz, 2H), 3.56 (m, 2H), 3.51-3.40 (m, 1H), 3.37 (dd, J=12.9, 3.1 Hz, 1H), 3.23 (dd, J=12.5, 9.5 Hz, 1H), 3.01 (br t, J=12.7 Hz, 2H), 2.39 (br t, J=13.1 Hz, 2H), 2.05-1.88 (m, 2H).

APCI-MS: m/z 477 and 479 [MH⁺].

LC (method A) R_{T}=5.67 min. 98.7% UV 220 nm.

LC (method B) R_{T}.=4.38 min. 99.4% UV 220 nm.

### EXAMPLE 26

### (2-{[(2S)-3-{[1-(4-chlorobenzyl)piperidin-4-yl]amino}-2-hydroxy-2-methylpropyl]oxy}-3,4-difluorophenyl)acetic acid bis(trifluoroacetate)

A mixture of [(2S)-2-methyloxiran-2-yl]methyl 3-nitrobenzenesulfonate (137 mg, 0.5 mmol), methyl (3,4-difluoro-2-hydroxyphenyl)acetate (26a, 101 mg, 0.5 mmol) and Cs₂CO₃ (196 mg, 0.6 mmol) in DMF (3 ml) was stirred at room temperature for 20 h. The reaction mixture was partitioned between ethyl acetate and water. The organic layer was dried over Na₂SO₄, filtered and the filtrate was concentrated in vacuo to give crude methyl (3,4-difluoro-2-{[(2S)-2-methyloxiran-2-yl]methoxy}phenyl)acetate which was taken into methanol (2 ml) and to this solution, 1-(4-chlorobenzyl)piperidin-4-amine (112 mg, 0.5 mmol) was added and it was stirred at 80° C. over night. The volatiles were removed in vacuo and the residue was dissolved in THF (3 ml), aqeuous NaOH (165 mg NaOH in 1.5 ml water) was added and stirred at 80° C. for 3 h, cooled to 0° C. and the pH was adjusted to 2 by addition of aqueous TFA. The volatiles were removed in vacuo and the residue was purified by HPLC (10-75% acetonitrile in water, 0.1% TFA) to give the title compound (170 mg).

(CD₃OD, 400 MHz): δ 7.50 (s, 4H); 7.06 (m, 1H); 6.99 (m, 1H); 4.31 (s, 2H); 4.17 (d, J=9.4 Hz, 1H); 4.07 (d, J=9.7 Hz, 1H); 3.74 (d, J=15.7 Hz, 1H); 3.61 (m, 3H); 3.49 (m, 1H); 3.41 (d, J=12.7 Hz, 1H); 3.22 (d, J=12.7 Hz, 1H); 3.10 (t, J=12.9 Hz, 2H); 2.43 (m, 2H); 2.05 (m, 2H); 1.40 (s, 3H).

APCI-MS: m/z 483 (MH⁺).

### Methyl (3,4-difluoro-2-hydroxyphenyl)acetate (26a)

To a cold (ice-water bath) solution of LiAlH₄ in THF (7.5 ml, 7.5 mmol) was added a solution of 3,4-difluoro-hydroxybenzoic acid (522 mg, 3 mmol) in THF (5 ml) dropwise. After addition was completed, ice-bath was removed and the reaction mixture was stirred at room temperature over night. The reaction mixture was cooled in an ice-bath and dilute aqueous HCl (10 ml) was added carefully. The ice-bath was removed and after 10 min at room temperature, extracted with ethyl acetate. The organic layer was washed with aqueous NaHCO₃ and water respectively. The organic layer was dried over Na₂SO₄, filtered and the filtrate was concentrated in vacuo to give crude 2,3 difluoro-6(hydroxymethyl)phenol (390 mg) which was taken into acetonitrile (10 ml) and triphenylphosphine hydrogen bromide (827 mg, 2.41 mmol) was added, stirred at 80° C. over night, cooled to room temperature and the volatiles were removed in vacuo to give crude (3,4-difluoro-2-hydroxybenzyl)(triphenyl)phosphonium bromide (1.1 g) which was taken into CH₂Cl₂ (10 ml) and to this suspension Et₃N (1 ml) was added. To this clear solution, methyl chlorocarbonate (227 mg, 2.4 mmol) in CH₂Cl₂ (2 ml) was added slowly at room temperature. After addition was completed the reaction mixture was stirred at room temperature over night, partitioned between ethyl acetate and water. The organic layer was dried over Na₂SO₄, filtered and the filtrate was concentrated in vacuo. The residue was purified by silica gel flash chromatography (0-1.5% methanol in CH₂Cl₂) to give the subtitle compound (267 mg).

¹H-NMR (DMSO-d₆, 400 MHz): δ 10.40 (br.s, 1H); 6.96 (m, 1H); 6.79 (m, 1H); 3.61 (s, 2H); 3.59 (s, 3H).

### EXAMPLE 27

### 2-(2-{[(2S)-3-{[1-(4-Chlorobenzyl)piperidin-4-yl]amino}-2-hydroxypropyl]oxy}phenyl)propanoic acid bis(trifluoroacetate) (salt)

The title compound was prepared according to the procedure described in Example 1 starting from 1-(4-chlorobenzyl)piperidin-4-amine (29 mg, 130 μmol) and methyl 2-{2-[(2S)-oxiran-2-ylmethoxy]phenyl}propanoate (27a, 31 mg, 130 μmol). Yield: 33 mg (37%) as a colourless powder.

¹H NMR (400 MHz, CD₃OD) δ 7.51 (s, 4H), 7.24 (m, 2H), 6.96 (m, 2H), 4.34 (s, 2H), 4.26 (m, 1H), 4.10 (ddd, J=11.3, 9.8, 4.3 Hz, 1H), 4.00 (m, 1H), 3.91 (sextet, J=7.1 Hz, 1H), 3.63 (br.d, J=12.7 Hz, 2H), 3.52 (m, 1H), 3.40 (dt, J=13.0, 3.5 Hz, 1H), 3.29-3.20 (m, 1H), 3.14 (br.t, J=12.4 Hz, 2H), 2.46 (m, 2H), 2.04 (br. quintet, J=12.8 Hz, 2H), 1.44 (dd, J=7.1, 2.3 Hz, 3H).

APCI-MS m/z: 447 [MH⁺].

### Methyl 2-{2-[(2S)-oxiran-2-ylmethoxy]phenyl}propanoate (27a)

The subtitled compound was prepared according to the protocol described for 1a starting from methyl 2-(2-hydroxyphenyl)propanoate (33 mg, 180 μmol) (as described by A. Padwa, et al., J. Am. Chem. Soc. 1976, 98, 3555-3564) and (2S)-oxiran-2-ylmethyl 3-nitrobenzenesulfonate (47 mg, 180 μmol. Yield: 31 mg (74%) as red oil.

### EXAMPLE 28

### {2-[2-({[1-(4-chlorobenzyl)piperidin-4-yl]amino}methyl)-2-hydroxybutoxy]phenyl}acetic acid

The title compound was prepared according to the procedure described in Example 1 starting from 1-(4-chlorobenzyl)piperidin-4-amine (40 mg, 180 μmol) and methyl {4-chloro-2-[2-ethyloxiran-2-ylmethoxy]phenyl}acetate (28a, 47 mg, 170 μmol). Yield: 21 mg (17%) as a colourless powder.

¹H NMR (400 MHz, CD₃OD): δ 7.48 (m, 4H), 7.30-7.22 (m, 2H), 7.00-6.94 (m, 2H), 4.25-4.13 (m, 2H), 4.02 (d, J=9.9 Hz, 1H), 3.95 (d, J=9.7 Hz, 1H), 3.62 (d, J=1.9 Hz, 2H), 3.57-3.43 (m, 3H), 3.39 (d, J=12.7 Hz, 1H), 3.12 (d, J=12.6 Hz, 1H), 2.93 (s, 2H), 2.36 (s, 2H), 1.96 (d, J=26.7 Hz, 2H), 1.76 (dq, J=25.6, 7.3 Hz, 2H), 1.01 (t, J=7.5 Hz, is 3H).

LC (method A) rt=5.96 min. 96% UV 220 nm.

LC (method B) rt=4.95 min. 97% LTV 220 nm.

### Methyl {2-[(2-ethyloxiran-2-yl)methoxy]phenyl}acetate (28a)

To an ice cold solution of methyl {2-[(2-ethylprop-2-enyl)oxy]phenyl}acetate (408 mg, 1.74 mmol) in dichloromethane (5 ml) m-chloro-perbenzoicacid (515 mg, 2.09 mmol) were added. Stirring was continued while the mixture was allowed to slowly warm up to ambient temperature. Dilution with ethylacetate, consecutive washing with aquous solutions of saturated sodium sulfite, 5 proc. sodium hydroxide, saturated sodium bicarbonate and evaporation of the organic solvent afforded a crude subtitle compound which was purified by flash chromatography (heptane/ethyl acetate=1:1). Yield: 318 mg (1.27 mmol).

¹H NMR (400 MHz, CDCl₃) δ 7.23 (m, 2H), 6.95 (td, J=7.4, 0.9 Hz, 1H), 6.86 (d, J=8.1 Hz, 1H), 4.03 (q, J=10.4 Hz, 2H), 3.70 (s, 3H), 2.83 (d, J=4.6 Hz, 1H), 2.76 (d, J=4.6 Hz, 1H), 1.89 (quintet, J=7.4 Hz, 1H), 1.75 (dq, J=14.6, 7.4 Hz, 1H), 1.00 (t, J=8.5 Hz, 3H).

### Methyl {2-[(2-ethylprop-2-enyl)oxy]phenyl}acetate (28b)

2-Ethyl-2-propen-1-ol (183 mg, 2.18 mmol), diethyl azodicarboxylate (454 mg, 2.61 mmol) and triphenylphosphine (685 mg, 2.61 mmol) were dissolved in THF (5 ml). Methyl 2-hydroxyphenylacetate (362 mg, 218 mmol) was added at 0° C. and the mixture was stirred over night and allowed to slowly warm up to ambient temperature. After dilution with brine (30 ml) and extraction with ethyl acetate 2×30 ml) the organic phase was dried over sodium sulfate. Evaporation of the solvent and flash chromatography on silica gel (heptane/ethyl acetate=2:1) 415 mg (81%) of the subtitle compound were obtained as a colourless oil.

¹H NMR (300 MHz, CDCl₃) δ 7.21 (ddd, J=9.6, 7.8, 1.7 Hz, 2H), 6.93 (td, J=7.5, 1.1 Hz, 1H), 6.87 (d, J=8.2 Hz, 1H), 5.11 (quintet, J=1.2 Hz, 1H), 4.98 (quintet, J=1.3 Hz, 1H), 4.48 (s, 2H), 3.68 (s, 3H), 2.16 (q, J=9.7 Hz, 2H), 1.11 (t, J=7.4 Hz, 3H).

### EXAMPLE 29

### (2-{[(2S)-3-{[(2R*,5S*)-1-(4-chlorobenzyl)-2,5-dimethylpiperidin-4-yl]amino}-2-hydroxy-2-methylpropyl]oxy}phenyl)acetic acid bis(trifluoroacetate)

A mixture of methyl (2-{[(2S)-2-methyloxiran-2-yl]methoxy}phenyl)acetate (3b, 9.5 mg, 0.04 mmol) and (2R*,5S*)-1-(4-chlorobenzyl)-2,5-dimethylpiperidin-4-amine (29a, 10 mg, 0.04 mmol) in methanol (1 ml) was stirred at 80° C. over night. The volatiles were removed in vacuo and the residue was dissolved in THF (1.5 ml), aqueous NaOH (165 mg NaOH in 0.6 ml water) was added, stirred at 80° C. for 3 h, cooled in an ice-bath and the pH was adjusted to 2 by addition of aqueous TFA. The volatiles were removed in vacuo and the residue was purified by HPLC (10-75% acetonitrile in water, 0.1% TFA) to give the title compound (12 mg).

APCI-MS: m/z 475 (MH⁺)

### (2R*,5S*)-1-(4-Chlorobenzyl)-2,5-dimethylpiperidin-4-amine (29a)

To a suspension of (2R*,5S*)-1-(4-chlorobenzyl)-2,5-dimethylpiperidin-4-one oxime (29b, 100 mg, 0.374 mmol) in diethyl ether (2 ml) was added a solution of LiAlH₄ in diethyl ether (2 ml, 2 mmol) slowly at 0° C. After addition was completed, ice-bath was removed and the reaction mixture was stirred at room temperature over night, cooled to 0° C., water (2 ml) was added slowly, pH was adjusted to 2 by addition of aqueous TFA and after 20 min at room temperature, extracted with ethyl acetate. The organic layer was dried over Na₂SO₄, filtered and the filtrate was concentrated in vacuo. The residue was purified by flash chromatography on silica gel (0-5% methanol in dichloromethane, 0.2% NH₄OH) to give the subtitle compound (10 mg).

APCI-MS: m/z 253 (MH⁺).

### (2R*,5S*)-1-(4-Chlorobenzyl)-2,5-dimethylpiperidin-4-one oxime (29b)

(2R*,5S*)-2,5-Dimethylpiperidin-4-one oxime (171 mg, 1.2 mmol) and 4-chlorobenzyl chloride (193 mg, 1.2 mmol) in DMF (1 ml) was stirred at 80° C. for 70 min, cooled to room temperature, partitioned between ethyl acetate and saturated aqueous NaHCO₃. The organic layer was washed with water, dried over Na₂SO₄, filtered and the filtrate was concentrated in vacuo. The residue was purified by flash chromatography on silica gel (0-2.5% methanol in dichloromethane, 0.2% NH₄OH) to give subtitle compound (207 mg).

¹H-NMR (CDCl₃, 400 MHz): δ 7.30 (s, 4H); 3.94 (d, J=13.6 Hz, 1H); 3.18 (d, J=13.6 is Hz, 1H); 3.14 (dd, J=3.7, 14.1 Hz, 1H); 2.80 (dd, J=4.6, 11.3 Hz, 1H); 2.5 (m, 2H); 1.97 (dd, J=9.9, 14.1 Hz, 1H); 1.89 (dd, J=10.1, 11.3 Hz, 1H); 1.19 (d, J=6.2 Hz, 3H); 1.00 (d, J=6.7 Hz, 3H).

APCI-MS: m/z 267 (MH⁺).

### EXAMPLE 30

### (2-{[(2S)-3-{[1-(4-chloro-2-methylbenzyl)piperidin-4-yl]amino}-2-hydroxypropyl]oxy}phenyl)acetic acid bis(trifluoroacetate) (salt)

The title compound was prepared according to the procedure described in Example 1 starting from 1-(4-chloro-2-methylbenzyl)piperidin-4-amine (24 mg, 100 μmol) and methyl {2-[(2S)-2-methyloxiran-2-ylmethoxy]phenyl}acetate (17a, 22 mg, 100 μmol). Yield: 25 mg (37%) as a colourless powder.

¹H NMR. (400 MHz, CD₃OD) δ 7.46 (d, J=8.3 Hz, 1H), 7.39 (s, 1H), 7.32 (dd, J=8.2, 1.9 Hz, 1H), 7.24 (m, 2H), 6.95 (m, 2H), 4.36 (s, 2H), 4.26 (m, 2H), 4.13 (dd, J=9.9, 4.4 Hz, 1H), 4.01 (dd, J=9.8, 6.3 Hz, 1H), 3.67 (s, 1H), 3.62 (d, J=4.4 Hz, 3H), 3.51 (m, 2H), 3.39 (dd, J=12.7, 3.0 Hz, 1H), 3.28-3.13 (m, 3H), 2.45 (s, 3H), 2.41 (br.d, J=12.9 Hz, 2H), 2.03 (quintet, J=17.7 Hz, 2H).

APCI-MS m/z: 447 [MH⁺].

### EXAMPLE 31

### 2-(2-{[(2S)-3-{[1-(4-chlorobenzyl)piperidin-4-yl]amino}-2-hydroxy-2-methylpropyl]oxy}phenyl)propanoic acid bis(trifluoroacetate) (salt)

The titled compound was prepared according to the procedure described in Example 1 starting from 1-(4-chlorobenzyl)piperidin-4-amine (65 mg, 290 μmol) and methyl 2-(2-{[(2S)-2-methyloxiran-2-yl]methoxy}phenyl)propanoate (31a, 72 mg, 290 μmol). Yield: 5 mg (3%) as a colourless powder.

¹H NMR (400 MHz, CD₃OD): δ 7.51 (s, 4H), 7.26 (m, 2H), 6.97 (m, 2H), 4.33 (s, 2H), 4.01-3.86 (m, 3H), 3.61 (br.d, J=12.4 Hz, 2H), 3.48 (m, 1H), 3.40 (d, J=12.7 Hz, 1H), 3.20 (d, J=12.7 Hz, 1H), 3.10 (br.t, J=12.3 Hz, 2H), 2.45 (br.d, J=12.6 Hz, 2H), 2.06 (m, 2H), 1.47 (dd, J=7.3, 3.7 Hz, 2H), 1.44 (d, J=2.5 Hz, 3H).

APCI-MS m/z: 461 [MH⁺].

### Methyl 2-(2-{[(2S)-2-methyloxiran-2-yl]methoxy}phenyl)propanoate (31a)

The subtitled compound was prepared according to the protocol described for 1a starting from methyl 2-(2-hydroxyphenyl)propanoate (53 mg, 290 μmol) (as described by A. Padwa, et al., J. Am. Chem. Soc. 1976, 98, 3555-3564) and [(2S)-2-methyloxiran-2-yl]methyl 3-nitrobenzenesulfonate (81 mg, 290 μmop. Yield: 72 mg (quant.) as brown oil.

### EXAMPLE 32

### 1-(2-{[(2S)-3-{[1-(4-Chlorobenzyl)piperidin-4-yl]amino}-2-hydroxypropyl]oxy}phenyl)cyclopropanecarboxylic acid bis(trifluoroacetate) (salt)

The title compound was prepared according to the procedure described in Example 1 starting from 1-(4-chloro-2-methylbenzyl)piperidin-4-amine (48 mg, 210 μmol) and methyl 1-{2-[(2S)-oxiran-2-ylmethoxy]phenyl}cyclopropanecarboxylate (32a, 53 mg, 210 mmol). Yield: 81 mg (56%) as a colourless powder.

¹H NMR (400 MHz, CD₃OD): δ 7.51 (s, 4H), 7.25 (m, 2H), 6.94 (m, 2H), 4.33 (s, 2H), 4.26 (m, 1H), 4.16 (dd, J=9.7, 4.1 Hz, 1H), 3.99 (dd, J=9.7, 6.7 Hz, 1H), 3.62 (br.d, 12.2 Hz, 2H), 3.48 (m, 1H), 3.35 (dd, J=12.6, 2.8 Hz, 1H, partially covered with the signal of solvent), 3.20 (dd, J=12.5, 10.0 Hz, 1H), 3.11 (br.t, J=12.1 Hz, 2H), 2.41 (br.t, J=13.1 Hz, 2H), 2.01 (br. quintet, J=18.2 Hz, 2H), 1.55 (m, 2H), 1.14 (m, 2H).

APCI-MS m/z: 459 [MH⁺].

### Methyl 1-{2-[(2S)-oxiran-2-ylmethoxy]-phenyl}cyclonropanecarboxylate (32a)

The subtitle compound was prepared according to the protocol described for 1a (stirring for 36 h) starting from methyl 1-(2-hydroxyphenyl)cyclopropanecarboxylate (32b, 52 mg, 300 μmol) and (25)-oxiran-2-ylmethyl 3-nitrobenzenesulfonate (78 mg, 300 μmol). Yield: 53 mg (72%) as brownish oil.

¹H NMR (400 MHz, CDCl₃): δ 7.29-7.19 (m, 2H), 6.94 (td, J=7.4, 1.1 Hz, 2H), 6.87 (d, J=8.3 Hz, 1H), 4.27 (dd, J=11.1, 2.8 Hz, 1H), 4.06 (dd, J=11.1, 5.0 Hz, 1H), 3.62 (s, 3H), 3.32 (m, 1H), 2.87 (m, 1H), 2.79 (dd, J=5.1, 2.7 Hz, 1H), 1.62 (q, J=3.6 Hz, 3H), 1.13 (dd, J=6.9, 3.9 Hz, 2H).

### Methyl 1-(2-hydroxyphenyl)cyclopropanecarboxylate (32b)

A stirred solution of methyl 1-(2-methoxyphenyl)cyclopropanecarboxylate (32c, 478 mg, 2.3 mmol) in dichloromethane (10 ml) was cooled to 0° C. Boron tribromide (1 M in solution in dichloromethane, 6.9 ml, 6.9 mmol) was added dropwise. The stirring was continued at 0° C. for 1 h. Then the mixture was quenched with several drops of methanol, and washed with aqueous NaHCO₃ (1 M, 10 ml). The organic layer was dried with sodium sulfate, and the solvent was removed in vacuo. The residue was purified by flash chromatography on silica gel (eluent: heptane/ethyl acetate, 2:1). Yield: 361 mg (82%).

¹H NMR. (400 MHz, CDCl₃): δ 7.27 (m, 2H), 6.95 (m, 2H), 5.76 (br.s, 1H), 3.73 (s, 3H), 1.74 (dd, J=7.0, 4.0 Hz, 2H), 1.27 (dd, J=7.0, 4.0 Hz, 2H).

### Methyl 1-(2-methoxyphenyl)cyclopropanecarboxylate (32c)

To a stirred solution of 1-(2-methoxyphenyl)cyclopropanecarboxylic acid (472 mg, 2.7 mmol) (as described by Sh. L. Mndzhoyan et al., Khim. Pharm. Zh. 1980, 14 (2), 40-45)) in methanol (10 ml) at 0° C. was added dropwise thionyl chloride (1 ml). Then the mixture was heated to reflux for 5 h. The solvent was removed in vacuo, the residue taken up with dichloromethane, washed with water and dried over sodium sulfate. Eveporation of solvent afforded brown oil, which was used without further purification. Yield: 478 mg (85%).

¹H NMR (400 MHz, CDCl₃): δ 7.28 (m, 2H), 7.21 (dd, J=7.4, 1.6 Hz, 2H), 6.90 (m, 2H), 3.84 (s, 3H), 3.61 (s, 3H), 1.61 (dd, J=7.1, 4.1 Hz, 3H), 1.12 (dd, J=7.1, 4.1 Hz, 21-1).

### EXAMPLE 33

### 1-(2-{[(2S)-3-{[1-(4-chlorobenzyl)piperidin-4-yl]amino}-2-hydroxy-2-methylpropyl]oxy}phenyl)cyclopropanecarboxylic acid bis(trifluoroacetate) (salt)

The title compound was prepared according to the procedure described in Example 1 starting from 1-(4-chloro-2-methylbenzyl)piperidin-4-amine (56 mg, 250 mop and methyl 1-(2-{[(2S)-2-methyloxiran-2-yl]methoxy}phenyl)cyclopropanecarboxylate (33a, 65 mg, 250 μmol). Yield: 100 mg (57%) as a colourless powder.

¹H NMR (400 MHz, CD₃OD): δ 7.50 (dd, J=16.9, 2.9 Hz, 4H), 7.26 (m, 2H), 6.94 (m, 2H), 4.32 (s, 2H), 4.00 (d, J=9.4 Hz, 1H), 3.88 (d, J=9.4 Hz, 1H), 3.61 (m, 2H), 3.43 (m, 1H), 3.29 (d, J=12.6 Hz, 1H, partially covered with the signal of solvent), 3.14 (d, J=12.6 Hz, 2H), 3.07 (d, J=12.4 Hz, 1H), 2.41 (d, J=13.6 Hz, 2H), 2.00 (quintet, J=11.8 Hz, 2H), 1.58 (m, 2H), 1.45 (s, 3H), 1.20 (m, 2H), 1.10 (m, 2H).

APCI-MS m/z: 473 [MH⁺].

### Methyl 1-(2-{[(2S)-2-methyloxiran-2-yl]methoxy}phenyl)cyclopropanecarboxylate (33a)

The subtitle compound was prepared according to the protocol described for 1a (stirring for 36 h) starting from methyl 1-(2-hydroxyphenyl)cyclopropanecarboxylate (32b, 52 mg, 300 pimp and [(2S)-2-methyloxiran-2-yl]methyl 3-nitrobenzenesulfonate (82 mg, 300 μmol). Yield: 65 mg (82%) as brownish oil.

¹H NMR (400 MHz, CDCl₃): δ 7.24 (m, 2H), 6.93 (td, J=7.5, 1.1 Hz, 1H), 6.83 (dd, J=8.2, 0.8 Hz, 1H), 4.04 (d, J=10.3 Hz, 1H), 3.97 (d, J=10.3 Hz, 1H), 3.61 (s, 3H), 2.85 (d, J=4.8 Hz, 1H), 2.72 (d, J=4.8 Hz, 1H), 1.63 (m, 2H), 1.47 (s, 3H), 1.14 (m, 3H).

### EXAMPLE 34

### (2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid bis(trifluoroacetate)

To a solution of (2R,5S)-1-(4-chlorobenzyl)-2,5-dimethylpiperidin-4-one oxime (34a, 60 mg, 0.22 mmol) in diethyl ether (3 ml) was added LiAlH₄ in diethyl ether (1.5 ml, 1.5 mmol) slowly at 0° C. After addition was completed the reaction mixture was kept in the refrezarator for 24 h. Methanol (0.5 ml) was added and after 15 min at room temperature, the volatiles were removed in vacuo. The residue was taken into a mixture of acetonitrile (3 ml) and water (1.5 ml) and the pH was adjusted to 2 by addition of TFA and the solution was subjected to HPLC (10-55% acetonitrile in water, 0.1% TWA) to give intermediate (2R,5S)-1-(4-chlorobenzyl)-2,5-dimethylpiperidin-4-amine (50 mg) which was taken into methanol (1.5 ml), Et₃N (0.056 ml) was added followed by methyl {4-fluoro-2-[(2S)-oxiran-2-ylmethoxy]phenyl}acetate (prepared in example 1) (25.4 mg, 0.1 mmol) and the mixture was stirred at 65° C. over night. The volatiles were removed in vacuo and the residue was subjected to HPLC (10-75% acetonitrile in water, 0.1% TFA) to give methyl (2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetate (26 mg) which was dissolve in THF (3 ml), aqueous NaOH (165 mg NaOH in 1.5 ml water) was added and the reaction mixture was stirred at 77° C. for 3 h, cooled to 0° C. and the pH was adjusted to 2 by addition of aqueous TFA. The volatiles were removed in vacuo and the residue was purified by HPLC (10-70% acetonitrile in water, 0.1% TFA) to give the title compound 34 (15 mg).

APCI-MS: m/z 493 (MH⁺).

### (2R,5S)-1-(4-Chlorobenzyl)-2,5-dimethylpiperidin-4-one oxime (34a)

(2R*,5S*)-1-(4-Chlorobenzyl)-2,5-dimethylpiperidin-4-one oxime (29b) (165 mg) was subjected to HPLC (Chiral pak. AD; isohexane/ethanol=97:3) to yield a compound (2R,5S*)-1-(4-chlorobenzyl)-2,5-dimethylpiperidin-4-one oxime (65 mg; R_{T}=21.1 min) as the second eluated isomer.

¹H-NMR (CDCl₃, 400 MHz) δ 7.37 (s, 1H); 7.27 (s, 4H); 3.93 (d, J=13.6 Hz, 1H); 3.17 (d, J=13.8 Hz, 1H); 3.13 (dd, J=3.7, 14.3 Hz, 1H); 2.79 (dd, J=4.6, 11.5 Hz, 1H); 2.55 (m, 1H); 2.45 (m, 1H); 1.97 (dd, J=9.9, 14.3 Hz, 1H); 1.90 (dd, J=9.9, 11.5 Hz, 1H); 1.19 (d, J=6.2 Hz, 3H); 1.08 (d, J=6.5 Hz, 3H).

APCI-MS: m/z 267 (MH⁺).

### (2S,5R)-1-(4-Chlorobenzyl)-2,5-dimethylpiperidin-4-one oxime (34b)

The separation described for 34a afforded the subtitle compound 34b as first eluated isomer (63 mg; R_{T}=15.6 min). The ¹H NMR spectrum is identical with the spectrum given for 34a.

### EXAMPLE 35

### (2-{[(2S)-3-{[(3S*,4R*)-1-(4-Chlorobenzyl)-3-methylpiperidin-4-yl]amino}-2-hydroxy-2-methylpropyl]oxy}-4-fluorophenyl)acetic acid bis(trifluoroacetate)

A mixture of {4-fluoro-2-[(2S)-oxiran-2-ylmethoxy]phenyl}acetate (prepared in Example 1) (64 mg, 0.251 mmol) and (3S*,4R*)-1-(4-chlorobenzyl)-3-methylpiperidin-4-amine (35a, 60 mg, 0.251 mmol) in methanol (2 ml) was stirred at 70° C. over night. The volatiles were removed in vacuo and the residue was dissolved in THF (3 ml), aqueous NaOH (165 mg NaOH in 1.5 ml water) was added and the reaction mixture was stirred at 80° C. for 3 h, cooled to 0° C. and the pH was adjusted to 2 by addition of aqueous TFA. The volatiles were removed in vacuo and the residue was purified by HPLC (10-70% acetonitrile in water, 0.1% TFA) to give title compound 35 (69 mg).

¹H-NMR (CD₃OD, 400 MHz): δ 7.50 (s, 4H); 7.20 (m, 1H); 6.80 (m, 1H); 6.70 (m, 1H); 4.21 (m, 2H); 4.00 (d, J=9.4 Hz, 1H); 3.90 (d, J=9.4 Hz, 1H); 3.60 (s, 2H); 3.50 (m, 2H); 3.32 (m, 2H); 3.19-2.93 (m, 3H); 2.68 (m, 1H); 2.20 (m, 2H); 1.43 (s, 3H); 1.22 (d, J=7.2 Hz, 1.32H); 1.17 (d, J=7.4 Hz, 1.68H).

APCI-MS: m/z 479 (MH⁺).

### (3S*,4R*)-1-(4-Chlorobenzyl)-3-methylpiperidin-4-amine (35a)

To a suspension of 1-(4-chlorobenzyl)-3-methylpiperidin-4-one-oxime (35b, 2g, 7.91 mmol) in dietheyl ether (50 ml) was added LiAlH₄ in diethyl ether (10 ml, 10 mmol) slowly at 0° C. After addition was completed ice-bath was removed ans stirred at room temperature over night. An additional 10 ml of LiAlH₄ in diethyl ether (10 mmol) was added followed by 5 ml of THF and the reaction mixture was stirred at romm temperature for 6 h, cooled to 0° C., MeOH (3 ml) was added slowly and ice-bath was removed, partitioned between ethyl acetate and saturated aqueous NH₄Cl. The organic phase was dried over Na₂SO₄, filtered and the filtrate was concentrated in vacuo. The residue was purified by silica gel flash chromatography (0-8% methanol in dichloromethane, 0.2% NH₄OH) to give cis isomer (3S*,4R*)-1-(4-Chlorobenzyl)-3-methylpiperidin-4-amine (35a, 297 mg), trans isomer (3S*,4S*)-1-(4-Chlorobenzyl)-3-methylpiperidin-4-amine (100 mg) and a mixture of both isomers (890 mg).

¹H-NMR (CD₃OD, 400 MHz): δ7.38 (s, 4H); 3.49 (d. J=13.1 Hz, 1H); 3.42 (d, J=13.1 Hz, 1H); 2.84 (m, 1H); 2.48 (m, 1H); 2.33 (m, 3H); 1.88 (m, 1H); 1.66 (m, 2H); 0.96 (d, J=7.1 Hz, 3H).

APCI-MS: m/z 239 (MH⁺)

### 1-(4-Chlorobenzyl)-3-methylpiperidin-4-one-oxime (35b)

3-Methylpiperidin-4-one (1.34 g, 11.9 mmol) was dissolved in dichloromethane (30 ml), Et₃N (6.12 ml) was added followed by 4-chlorobenzyl bromide (2.46 g, 12 mmol) and the reaction mixture was stirred at room temperature over night, partitioned between dichloromethane and water. The organic layer was dried over Na₂SO₄, filtered and the filtrate was concentrated in vacuo. The residue was dissolved in pyridine (30 ml), NH₂OH HCl (1.53 g) was added and the mixture was stirred at room temperature over night, partitioned between ethyl acetate and water. The organic layer was washed with water, dried over Na₂SO₄, filtered and the filtrate was concentrated in vacuo to give 1-(4-chlorobenzyl)-3-methylpiperidin-4-one-oxime (2.1 g). ¹H-NMR (CDCl₃, 400 MHz): δ8.39 (br.s, 1H); 7.28 (s, 4H); 3.47 (m, 2H); 3.00 (m, 1H); 2.77 (m, 2H); 2.58 (m, 1H); 2.26 (m, 2H); 2.05 (dd, J=9.2, 11.0 Hz, 1H); 1.08 (d, J=6.6 Hz, 3H).

APCI-MS: m/z 253 (MH⁺)

### EXAMPLE 36

### (2-{[(2S)-3-{[(3S*,4S*)-1-(4-Chlorobenzyl)-3-methylpiperidin-4-yl]amino}-2-hydroxy-2-methylpropyl]oxy}-4-fluorophenyl)acetic acid bis(trifluoroacetate)

A mixture of {4-fluoro-2-[(2S)-oxiran-2-ylmethoxy]phenyl}acetate (prepared in example 1) (64 mg, 0.251 mmol) and (3S*,4S*)-1-(4-chlorobenzyl)-3-methylpiperidin-4-amine (prepared in example 35a) (60 mg, 0.251 mmol) in methanol (2 ml) was stirred at 70° C. over night. The volatiles were removed in vacuo and the residue was dissolved in THF (3 ml), aqueous NaOH (165 mg NaOH in 1.5 ml water) was added and the reaction mixture was stirred at 80° C. for 3 h, cooled to 0° C. and the pH was adjusted to 2 by addition of aqueous TFA. The volatiles were removed in vacuo and the residue was purified by HPLC (10-70% acetonitrile in water, 0.1% TFA) to give title compound (69 mg).

¹H-NMR (CD₃OD, 400 MHz): δ7.50 (s, 4H); 7.22 (dd, J=6.9, 8.1 Hz, 1H); 6.79 (dd, 2.5, 10.8 Hz, 1H); 6.69 (m, 1H); 4.30 (m, 2H); 3.97 (m, 2H); 3.67-3.44 (m, 5H); 3.36 (d, J=12.7 Hz, 1H); 3.11 (dd, J=6.0, 12.7 Hz, 1H); 3.08 (m, 1H); 2.88 (t, J=12.0 Hz, 1H); 2.35 (m, 2H); 2.11 (m, 1H); 1.42 (s, 3H); 1.16 (d, J=6.5 Hz, 1.5H); 1.13 (d, J=6.5 Hz, 1.5H).

APCI-MS: m/z 479 (MH⁺).

### EXAMPLE 37

### 2-(2-{[(2S)-3-{[1-(4-Chlorobenzyl)piperidin-4-yl]amino}-2-hydroxy-2-methylpropyl]oxy}-4-fluorophenyl)propanoic acid

Methyl 2-{2-[((25)-3-[1-(4-chlorobenzyl)piperidin-4-yl]amino-2-hydroxy-2-methylpropyl)oxy]-4-fluorophenyl}propanoate (37a, 34 mg, 69 μmol) was dissolved in THF (0.60 ml), MeOH (0.60 ml), and H₂O (0.15 ml). An aqueous solution of LiOH (0.40 ml, 0.41 mmol) was added to ester 37a and the resulting mixture was stirred at room temperature for 18 h. The volatiles were removed in vacuo and the resulting residue was dissolved in CH₃CN before acidification by addition of AcOH. The crude material was purified by filtration through a C18-plug (Varian Mega Bond Elut C18) with CH₃CN in H₂O containing 2 g/L NH₄OAc as eluent. The title compound was obtained as a white solid in 29 mg (85%) after freeze-drying.

¹H-NMR (CD₃OD, 400 MHz): δ 7.33 (s, 4H), 7.24 (dd, J=8.3, 7.0 Hz, 1H), 6.69 (m, 2H), 3.93 (s, 1H), 3.90 (d, J=6.6 Hz, 1H), 3.70 (dq, J=22.0, 7.6 Hz, 1H), 3.55 (s, 2H), 3.39 (t, J=12.6 Hz, 1H), 3.13 (m, 1H), 3.02 (m, 3H), 2.20-2.08 (m, 4H), 1.87-1.66 (m, 2H), 1.44 (ddd, J=14.9, 7.0, 5.8 Hz, 3H).

APCI-MS: m/z 479 [MH⁺].

LC (method A) R_{T}=6.47 min. 98.7% UV 220 nm.

LC (method B) R_{T}=6.10 min. 99.4% UV 220 nm.

### Methyl 2-{2-[((2S)-3-{[1-(4-chlorobenzyl)piperidin-4-yl]amino}-2-hydroxy-2-methylpropyl)oxy]-4-fluorophenyl}propanoate (37a)

The crude methyl 2-(4-fluoro-2-{[(2S)-2-methyloxiran-2-yl]methoxy}phenyl)propanoate (37b, 47 mg, 0.17 mmol) was dissolved in MeOH (1.2 ml) and then added to 1-(4-chlorobenzyl)piperidin-4-amine (41 mg, 0.18 mmol). The resulting mixture was stirred at 60° C. for 20 h. The solvent was removed in vacuo and the residue purified by flash chromatography to give 46 mg (61% from 37c) of a diastereomeric mixture of subtitle compound 37a.

¹H-NMR (CD₃OD, 400 MHz): δ 7.30 (m, 4H), 7.19 (m, 1H), 6.76 (dd, J=11.1, 2.4 Hz, 1H), 6.66 (td, J=8.4, 2.3 Hz, 1H), 3.98 (m, 1H), 3.90 (dd, J=11.6, 9.1 Hz, 1H), 3.79 (dd, J=11.7, 9.0 Hz, 1H), 3.63 (s (one diastereomer), 3H), 3.62 (s (other diastereomer), 3H), 3.49 (s, 2H), 2.85 (m, 3H), 2.73 (dd, J=12.0, 4.2 Hz, 1H), 2.57-2.47 (m, 1H), 2.06 (br t, J=11.6 Hz, 2H), 1.91 (m, 2H), 1.47 (m, 2H), 1.43 (d, J=7.3 Hz, 3H), 1.31 (d, J=2.3 Hz, 3H).

APCI-MS: m/z 493 [MH⁺].

### Methyl 2-(4-fluoro-2-{[(2S)-2-methyloxiran-2-yl]methoxy}phenyl)propanoate (37b)

Methyl 2-(4-fluoro-2-{[(4R)-2,2,4-trimethyl-1,3-dioxolan-4-yl]methoxy}phenyl)propanoate (37c, 50 mg, 0.15 mmol) was dissolved in AcOH (0.45 m ml). HBr (80 mg, 0.46 mmol, 45% in AcOH) was added to dioxolan 37c and the resulting mixture was stirred at 30° C. for 4 h. NaOMe (solid and 1 M solution) and MeOH were added in portions until a pH of 12. The reaction mixture was neutralized by addition of AcOH and then partitioned between EtOAc and H₂O. The organic phase was washed with H₂O (3×5 ml), dried over Na₂SO₄, filtered, and concentrated in vacuo to give the crude subtitle compound 37b as a solid.

APCI-MS: m/z 310 [MH⁺+CH₃CN].

### Methyl 2-(4-fluoro-2-{[(4R)-2,2,4-trimethyl-1,3-dioxolan-4-yl]methoxy}phenyl)propanoate (37c)

DEAD (190 μl, 1.2 mmol) and triphenyl phosphine (318 mg, 1.2 mmol) were added to a solution of methyl 2-(4-fluoro-2-hydroxyphenyl)propanoate (37d, 1.0 mmol, 200 mg) in THF (2.5 ml) at 0° C. This mixture was stirred for 10 min before addition of (R)-(2,2,4-trimethyl-1,3-dioxolan-4-yl)methanol as describe by Avenoza, A. Cativiela, C.; Peregrina, J. M.; Sucunza, D.; Zurbano M. M. Tetrahedron: Asymmetry, 2001, 1383-1388. (1.0 mmol, 175 mg, 84% purity). The resulting reaction mixture was stirred for 16 h while allowed to reach room temperature before heating to 50° C. for 5 h. After additional stirring at r.t the solvent was removed in vacuo and the residue dissolved in Et₂O (3 ml) and left at r.t over night. The formed crystals were removed by filtration and the filtrate was purified by flash chromatography with pentane, CH₂Cl₂, and Et₂O as eluent. Subtitle compound 37c was obtained in 59 mg (18%) as solid.

¹H-NMR (CD₃OD, 400 MHz): δ 7.18 (dt, J=0.2, 7.3 Hz, 1H), 6.76 (dd, J=11.0, 2.6 Hz, 1H), 6.66 (td, J=8.4, 2.4 Hz, 1H), 4.06 (dd, J=8.8, 1.1 Hz, 1H), 3.96 (m, 1H), 3.89 (d, J=9.0 Hz, 1H), 3.81 (m, 2H), 3.64 (s, 3H), 1.46-1.37 (m, 12H).

APCI-MS: m/z 327 [MH⁺].

### Methyl 2-(4-fluoro-2-hydroxyphenyl)propanoate (37d)

Methyl 2-(4-fluoro-2-methoxyphenyl)acetate (37e, 299 mg, 1.41 mmol) was dissolved in CH₂Cl₂ and cooled to −30° C. Boron tribromide (2.82 ml, 2.82 mmol) was added dropwise while maintaining the temperature below −20° C. The reaction was stirred for 35 min to before quenching with MeOH. The pH was adjusted to 7 by addition of NaHCO₃ (aq) and the two phases were then separated. The aqueous phase was extracted with CH₂Cl₂ (1×3 ml) and the combined organic phase dried over Na₂SO₄, filtered, and evaporated to give the subtitle compound 37d as a solid in 278 mg (99%).

¹H-NMR (CD₃OD, 400 MHz): δ 7.09 (m, 1H), 6.55-6.47 (m, 2H), 3.97 (q, J=7.2 Hz, 1H), 3.64 (s, 3H), 1.40 (d, J.=7.3 Hz, 3H).

### Methyl 2-(4-fluoro-2-methoxyphenyl)propanoate (37e)

Methyl 2-(4-fluoro-2-methoxyphenyl)acetate (prepared as described in EP 1340761) (500 mg, 2.52 mmol) was dissolved in THF (2 ml) and cooled to 0° C. LDA (1.89 ml, 3.78 mmol) was added dropwise and the resulting mixture was stirred for 30 min and then cooled to −78° C. Methyl iodide (251 μl, 4.04 mmol) dissolved in DMPU (260 μl) and THF (0.5 ml) was added and the reaction mixture was stirred for 5 h at −10 to −30° C. The reaction was quenched by addition of MeOH, neutralized by addition of HCl (5 M, aq), and diluted with EtOAc. The aqueous phase was extracted with EtOAc (3×3 ml) and the combined organic phase was then washed with H₂O and brine, dried over Na₂SO₄, filtered and evaporated. The crude material was purified by reversed phase HPLC (Kromasil column, 100-5-C₁₈, 250×20 mm, 15-75% CH₃CN in H₂O containing 0.1% TFA). The subtitle compound 37e was obtained in 308 mg (58%).

¹H-NMR (CD₃OD, 400 MHz): δ 7.16 (dd, J=8.4, 6.6 Hz, 1H), 6.74 (dd, J=11.0, 2.5 Hz, 1H), 6.63 (td, J=8.4, 2.5 Hz, 1H), 3.96 (q, J=7.2 Hz, 1H), 3.80 (s, 3H), 3.80 (s, 3H), 1.39 (d, J=7.3 Hz, 3H).

### EXAMPLE 38

### 2-[2-(3-{[1-(4-chlorobenzyl)piperidin-4-yl]amino}propoxy)phenoxy]acetic acid bis(trifluoroacetate)

To a solution of tert-butyl[1-(4-chlorobenzyl)piperidin-4-yl](3-hydroxypropyl)carbamate (prepared in example 7) (422 mg, 1.1 mmol), pyrocatechol (145 mg, 1.32 mmol) and Ph₃P (346 mg, 1.32 mmol) in THF (3 ml) was added a solution of DEAD (230 mg, 1.32 mmol) in THF (2 ml) slowly at room temperature. After addition was completed the reaction mixture was stirred at room temperature for 24 h. The volatiles were removed in vacuo and the residue was purified by silica gel flash chromatography (0-1.5% methanol in dichloromethane, 0.2% NH₄OH) to give tert-butyl[1-(4-chlorobenzyl)piperidin-4-yl][3-(2-hydroxyphenoxy)propyl]carbamate (800 mg, contaminated with large quantity of triphenylphosphine oxide). One portion (135 mg) of this material was dissolved in DMF (2 ml), Cs₂CO₃ (326 mg, 1 mmol) and methyl 2-bromopropanoate (0.112 ml) were added. The reaction mixture was stirred at room temperature for 7 h, partitioned between ethyl acetate and water. The organic layer was dried over Na₂SO₄, filtered and the filtrate was concentrated in vacuo. The residue was dissolved in THF (3 ml), aqueous NaOH (165 mg, NaOH in 1.5 ml water) was added and stirred at 80° C. for 3 h, cooled to 0° C. and the pH was adjusted to 2 by addition of TFA. The volatiles were removed in vacuo and the residue was purified by HPLC (10-80% acetonitrile in water, 0.1% TFA) to give the title compound (27 mg).

¹H-NMR (CD₃OD, 400 MHz): δ7.49 (s, 4H); 7.05-6.92 (m, 4H); 4.79 (q, 1H); 4.28 (s, 2H); 4.20 (m, 2H); 3.56 (br.d, J=12.7 Hz, 2H); 3.49 (m, 1H); 3.36 (t, J=6.7 Hz, 2H); 3.01 (br.t, J=12.0 Hz, 2H); 2.38 (m, 2H); 2.20 (m, 2H); 1.98 (m, 2H); 1.58 (d, J=6.9 Hz, 3H).

APCI-MS: m/z 447 (MH⁺).

### EXAMPLE 39

### [2-(3-{[1-(4-chlorobenzyl)piperidin-4-yl]amino}propoxy)phenoxy]acetic acid bis(trifluoroacetate)

To a solution of tert-butyl[1-(4-chlorobenzyl)piperidin-4-yl][3-(2-hydroxyphenoxy)propyl]carbamate (Example 38) (254 mg) in DMF (2.5 ml) Cs₂CO₃ (326 mg, 1 mmol) and methyl bromoacetate (0.096 ml) were added. The reaction mixture was stirred at room temperature for over night, partitioned between ethyl acetate and water. The organic layer was dried over Na₂SO₄, filtered and the filtrate was concentrated in vacuo. The residue was dissolved in THF (3 ml), aqueous NaOH (165 mg, NaOH in 1.5 ml water) was added and stirred at 80° C. for 3 h, cooled to 0° C. and the pH was adjusted to 2 by addition of TFA. The volatiles were removed in vacuo and the residue was purified by HPLC (10-75% acetonitrile in water, 0.1% TFA) to give the title compound (30 mg).

¹H-NMR (CD₃OD, 400 MHz): δ87.50 (s, 4H); 7.06-6.94 (m, 4H); 4.70 (s, 2H); 4.32 (s, 2H); 4.21 (t, J=5.5 Hz, 2H); 3.64-3.50 (m, 3H); 3.40 (t, J=6.7 Hz, 2H); 3.10 (t, J=12.9 Hz, 2H); 2.39 (br.d, J=13.3 Hz, 2H); 2.20 (m, 2H); 2.01 (m, 2H).

APCI-MS: m/z 433 (MH⁺).

### EXAMPLE 40

### (2-{[(2S)-3-{[1-(4-chlorobenzyl)-2,2,6,6-tetramethylpiperidin-4-yl]amino}-2-hydroxy-2-methylprppyl]oxy}-4-fluorophenyl)acetic acid bis(trifluoroacetate)

{4-Fluoro-2-[(2S)-oxiran-2-ylmethoxy]phenyl}acetate (Example 1) (5.4 mg, 0.021 mmol) and 1-(4-chlorobenzyl)-2,2,6,6-tetramethylpiperidin-4-amine (40a, 6 mg, 0.021 mmol) were taken into methanol (1 ml) and stirred at 80° C. over night. The volatiles were removed in vacuo and the residue was dissolved in THF (3 ml), aqueous NaOH (165 mg NaOH in 1.5 ml water) was added and the mixture was stirred at 86° C. for 3.5 h, cooled to 0° C. and pH was adjusted to 2 by addition of aqueous TFA. The volatiles were removed in vacuo and the residue was purified by HPLC (10-85% acetonitrile in water, 0.1% TFA) to give the title compound (2 mg).

¹H-NMR (CD₃OD, 400 MHz): δ 7.48 (br.d, J=8.5 Hz, 2H); 7.32 (br.d, J=8.1 Hz, 2H); 7.23 (dd, J=6.5, 8.3 Hz, 1H); 6.81 (dd, J=2.5, 11.0 Hz, 1H); 6.70 (m, 1H); 4.12 (br, s, 2H); 4.01 (d, J=9.5 Hz, 1H); 3.94 (d, J=9.5 Hz, 1H); 3.74-3.54 (m, 3H); 3.41 (d, J=12.9 Hz, 1H); 3.20 (d, J=12.9 Hz, 1H); 2.21 (m, 2H); 1.92 (m, 2H); 1.44 (s, 3H); 1.38 (s, 6H); 1.20 (s, 6H).

APCI-MS: m/z 521 (MH⁺).

### 1-(4-Chlorobenzyl)-2,2,6,6-tetramethylpiperidin-4-amine (40a)

To a cold (ice-waterbath) solution of 1-(4-chlorobenzyl)-2,2,6,6-tetramethylpiperidin-4-one-oxime (40b, 180 mg, 0.61 mmol) was added 1 M solution of LiAlH₄ in THF (2 ml, 2 mmol) slowly. After addition was completed the reaction mixture was stirred at room temperature for 24 h, cooled to 0° C. and saturated aqueous NH₄Cl (10 ml) was added slowly. After addition was completed the reaction mixture was stirred at room temperature for 10 min, extracted with ethyl acetate. The organic layer was dried over Na₂SO₄, filtered and the filtrate was concentrated in vacuo. The residue was purified by silica gel flash chromatography (0-1.5% methanol in dichloromethane, 0.2% NH₄OH) to give subtitle compound (70 mg).

APCI-MS: m/z 281 (MH⁺).

### 1-(4-Chlorobenzyl)-2,2,6,6-tetramethylpiperidin-4-one-oxime (40b)

To a solution of 1-(4-chlorobenzyl)-2,2,6,6-tetramethylpiperidin-4-one (40c, 196 mg, 0.7 mmol) in pyridine (6 ml) was added hydroxylamine hydrochloride (138 mg, 2 mmol) and the mixture was stirred at room temperature over night, partitioned between ethyl acetate and water. The organic layer was dried over Na₂SO₄, filtered and the filtrate was concentrated and coevaporated with toluene in vacuo to give subtitle compound (185 mg).

¹H-NMR (CDCl₃, 400 MHz): δ57.41 (m, 2H); 7.28 (m, 2H); 6.98 (br.s, 1H); 3.81 (s, 2H); 2.60 (br.s, 2H); 2.29 (s, 2H); 1.08 (s, 12H).

APCI-MS: m/z 295 (MH⁺).

### 1-(4-Chlorobenzyl)-2,2,6,6-tetramethylpiperidin-4-one (40c)

4-Chlorobenzyl bromide (1.02 g, 5 mmol) was added to a suspension of 2,2,6,6-tetramethylpiperidin-4-one (1.55 g, 10 mmol) and Cs₂CO₃ (3.26 g, 10 mmol) in DMF (10 ml) and the mixture was stirred at 90° C. for 8 h, another portion of 4-chlorobenzyl bromide (1.02 g, 5 mmol) was added and stirred over night. An additional portion of Cs₂CO₃ (3.25 g, 10 mmol) was added followed by 4-chlorobenzyl bromide (2.05 g, 10 mmol) and the reaction mixture was stirred at 90° C. over night, cooled to room temperature and partitioned between ethyl acetate and water. The organic layer was dried over Na₂SO₄, filtered and the filtrate was concentrated in vacuo. The residue was purified by silica gel flash chromatography (0-0.5% methanol in dichloromethane, 0.2% NH₄OH) to give subtitle compound (200 mg). ¹H-NMR. (CDCl₃, 400 MHz): δ57.43 (br.d, J=8.3 Hz, 2H); 7.31 (m, 2H); 3.91 (s, 2H); 2.43 (s, 4H); 1.17 (s, 12H).

APCI-MS: m/z 280 (MH⁺).

### EXAMPLE 41

### (2-{[(2S)-3-{[(2R,4S,55)-1-(4-chlorobenzyl)-2,5-dimethylpiperidin-4-yl]amino}-2-hydroxy-2-methylpropyl]oxy}-4-fluorophenyl)acetic acid bis(trifluoroacetate) (salt)

(2R,5S)-1-(4-Chlorobenzyl)-2,5-dimethylpiperidin-4-amine (41a, 32 mg, 125 μmol) and methyl {4-fluoro-2-[(2S)-oxiran-2-ylmethoxy]phenyl}acetate (1a, 32 mg, 125 μmol) were dissolved in ethanol (3.0 ml) and the mixture was stirred for 36 h at 80° C. The sovlent was removed in vacuum, and a mixture was purified by reverse phase HPLC (Kromasil column, 100-5-C₁₈, 250×20 mm) using acetonitrile (0.1% TFA) and H₂O (0.1% TFA). The following separation of the diastereomers by reverse phase HPLC (Purosphere column) using acetonitrile and H₂O (0.03% NH₃) afforded (2-{[(2S)-3-{[(2R,4S,58)-1-(4-chlorobenzyl)-2,5-dimethylpiperidin-4-yl]amino}-2-hydroxy-2-methylpropyl]oxy}-4-fluorophenypacetate (8 mg) and methyl (2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetate (3 mg, used in Example 42). Then (2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenypacetate was dissolved in ethanol (1 ml), and aqueous 2M NaOH (1 ml) was added. The mixture was stirred at 80° C. for 30 min. After cooling to room temperature the mixture was acidified with trifluoroacetic acid and concentrated under reduced pressure. The crude product was purified by reverse phase HPLC (Kromasil column, 100-5-C₁₈, 250×20 mm) using acetonitrile (0.1% TFA) and H₂O (0.1% TFA). After freeze-drying, 10 mg (11%) of the title compound 41 was obtained as a colourless powder.

¹H NMR (400 MHz, CD₃OD): δ 7.51 (s, 4H), 7.24 (t, J=7.4 Hz, 1H), 6.80 (dd, J=10.7, 2.2 Hz, 1H), 6.71 (td, J=8.4, 2.3 Hz, 1H), 4.71 (br.d, J=12.9 Hz, 1H), 4.09 (br.d, J=11.5 Hz, 1H), 3.96 (m, 2H), 3.62 (dd, J=23.4, 15.7 Hz, 2H), 3.43 (s, 1H), 3.35 (d, J=12.8 Hz, 1H), 3.24 (dd, J=12.8, 3.8 Hz, 1H), 3.13 (d, J=12.7 Hz, 1H), 2.77 (t, J=12.6 Hz, 1H), 2.34 (d, J=15.0 Hz, 1H), 2.33 (br.s, 1H), 2.07 (q, J=12.9 Hz, 1H), 1.61 (d, J=6.2 Hz, 3H), 1.44 (s, 3H), 1.09 (d, J=6.5 Hz, 3H).

APCI-MS m/z: 493 [MH⁺].

### (2R,5S)-1-(4-Chlorobenzyl)-2,5-dimethylpiperidin-4-amine (41a)

A stirred solution of (2R,5S)-1-(4-chlorobenzyl)-2,5-dimethylpiperidin-4-one oxime (34a, 164 mg, 610 μmol) in THF (10 ml) was cooled to 0° C. A solution of lithium aluminium hydride in tetrahydrofurane (1 M, 3 ml, 3 mmol) was added dropwise. The mixture was kept at 4° C. for 16 h, and then stirred at room temperature for 24 h, and quenched with methanol (0.5 ml). The inorganic precipitate was removed by filtration, and the product purified by reverse phase HPLC (Kromasil column, 100-5-C₁₈, 250×20 mm) using acetonitrile (0.1% TFA) and H₂O (0.1% TFA). The fractions containing product were combined and concentrated in vacuo. An aqueous Solution of NaHCO₃ (1 M, 10 ml) was added, and the mixture was extracted with ethyl acetate (2×25 ml). Drying with sodium to sultate and evaporation of solvent afforded colourless oil. Yield 67 mg (43%).

APCI-MS m/z: 253 [MH⁺].

### EXAMPLE 42

### (2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid bis(trifluoroacetate) (salt)

Methyl (2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetate (3 mg, 6 μmol, Example 41) was dissolved in ethanol (1 ml), and aqueous 2 M NaOH (1 ml) was added. The mixture was stirred at 80° C. for 30 min. After cooling to room temperature the mixture was acidified with trifluoroacetic acid and concentrated under reduced pressure. The crude product was purified by reverse phase HPLC (Kromasil column, 100-5-C₁₈, 250×20 mm) using acetonitrile (0.1% TFA) and H₂O (0.1% TFA). After freeze-drying, 5 mg (6%) of the title compound 42 was obtained as a colourless powder.

¹H NMR (400 MHz, CD₃OD): δ 7.50 (s, 4H), 7.24 (dd, J=8.1, 8.0 Hz, 1H), 6.80 (dd, J=10.8, 2.3 Hz, 1H), 6.70 (td, J=8.4, 2.4 Hz, 1H), 4.37 (br.s, 1H), 4.19 (br.s, 1H), 4.03 (d, J=9.2 Hz, 1H), 3.87 (d, J=9.0 Hz, 1H), 3.60 (m, 3H), 3.09 (m, 2H), 2.47 (m, 2H), 2.05 (s, 1H), 1.47 (d, J=6.4 Hz, 6H), 1.43 (s, 3H), 1.13 (d, J=7.1 Hz, 3H).

APCI-MS m/z: 493 [MH⁺].

### EXAMPLE 43

### 3-[2-(3-{[(2R,4S,5S)-1-(4-Chlorobenzyl)-2,5-dimethylpiperidin-4-yl]amino}propoxy)phenyl]propanoic acid

The title compound was prepared according to the procedure described in Example 37 starting from methyl 3-[2-(3-{[(2R,4S,5S)-1-(4-chlorobenzyl)-2,5-dimethylpiperidin-4-yl]amino}propoxy)phenyl]propanoate 43a. The crude product was purified by filtration through a C18-plug (Varian Mega Bond Elut C18) with CH₃CN in H₂O containing 2g/l NH₄OAc as eluent. The title compound was obtained as a white solid in 16 mg (99%) after freeze-drying.

¹H NMR (500 MHz, CD₃OD) δ 7.33 (s, 4H), 7.15 (m, 2H), 6.90 (d, J=8.9 Hz, 1H), 6.86 (t, J=7.5 Hz, 1H), 4.11 (t, J=6.4 Hz, 2H), 3.66 (s, 1H), 3.24-3.16 (m, 2H), 3.36 (m, 2H, partially covered with the signal of solvent), 2.90 (m, 2H), 2.77 (m, 1H), 2.43 (m, 2H), 2.24 (m, 1H), 2.15 (m, 1H), 1.94 (s, 2H), 1.85 (m, 1H), 1.51 (q, J=11.6 Hz, 1H), 1.30 (d, J=6.2 Hz, 3H), 0.99 (d, J=6.2 Hz, 3H).

APCI-MS: m/z 459 [MH⁺].

LC (method A) R_{T}=6.31 min. 98.3% UV 220 nm.

LC (method B) R_{T}=6.13 min. 96.0% UV 220 nm.

### Methyl 3-[2-(3-{[(2R,4S,5S)-1-(4-chlorobenzyl)-2,5-dimethylpiperidin-4-yl]amino}propoxy)phenyl]propanoate (43a)

A mixture of (2R,4S,5S)- and (2R,4R,5S)-1-(4-chlorobenzyl)-2,5-dimethylpiperidin-4-amine (41b, 37 mg, 0.15 mmol) was dissolved in DMF (1.5 ml) and methyl 3-(2-(3-chloropropoxy)phenyl)propanoate (43b, 38 mg, 0.15 mmol), K₂CO₃ (22 mg, 0.16 mmol), and KI (10 mg, 58 μmol) were added. This mixture was stirred at 65° C. for 24 h and then partitioned between EtOAc and H₂O. The organic phase was washed with H₂O, dried over Na₂SO₄, filtered and evaporated. The crude mixture of diastereomers were purified and separated by reversed phase HPLC (Kromasil column, 100-5-C₁₈, 250×20 mm, 5-95% CH₃CN in H₂O containing 1 ml/l NH₄OH). The subtitle compound 43a was obtained in 16 mg (23%).

¹H NMR (400 MHz, d₃-acetonitrile): δ 7.30 (m, 4H), 7.16 (m, 2H), 6.92 (d, J=8.1 Hz, 1H), 6.85 (td, J=7.4, 0.9 Hz, 1H), 4.06 (t, J=6.4 Hz, 2H), 4.02 (d, J=14.0 Hz, 2H), 3.60 (s, 3H), 2.99 (d, J=13.8 Hz, 1H), 2.89 (m, 2H), 2.69-2.55 (m, 3H), 2.22 (m, 1H), 2.05-1.97 (m, 3H), 1.87 (quintet d, J=6.4, 1.8 Hz, 2H), 1.62 (t, J=11.3 Hz, 1H), 1.32 (m, 1H), 1.14 (d, J=6.2 Hz, 3H), 1.04 (d, J=12.2 Hz, 1H), 0.80 (d, J=6.5 Hz, 3H).

APCI-MS: m/z 473 [MH⁺].

### Methyl 3-[2-(3-chloropropoxy)phenyl]propanoate (43b)

To a solution of methyl 3(2-hydroxyphenyl)propanoate (5 g, 27.74 mmol) and 1-bromo-3-chloropropane (5.24 g, 33.3 mmol) in DMF (75 ml) was added potassium carbonate (7.7 g, 55.5 mmol) and the mixture was stirred at 80° C. for 12 h. After cooling it was diluted with EtOAc and washed with water, dried over Na₂SO₄, filtered and concentrated. Purification by flash chromatography with heptane and EtOAc as eluent. The subtitle compound 43b was obtained in 3.17 g (44%).

### EXAMPLE 44

### 3-[2-(3-{[1-(3,4-Dichlorobenzyl)piperidin-4-yl]amino}propoxy)phenyl]propanoic acid bis(trifluoroacetate) (salt)

The mixture of 1-(3,4-dichlorobenzyl)piperidin-4-amine (44a) (1.36 g, 5.25 mmol), methyl 3-[2-(3-chloropropoxy)phenyl]propanate (1.28 g, 5.0 mmol) and potassiumcarbomate (1.45 g, 10.5 mmol) in DMF was stirred at 600° C. for 16 h. Cooled and diluted with EtOAc and washed with water and brine, dried over Na₂SO₄, filterd and concentrated in vacuo to a brown oil. The oil was hydrolized with 2.0M LiOH.H₂O:MeOH. The organic solvent was removed and the crude material adjusted to pH 1 with 2.0 M HCl. Washed with EtOAC and after drying the concentration in vacuo purified on HPLC to give 164 mg of the title compound.

¹H-NMR (CD₃OD, 400 MHz): δ 7.7 (d, J=9.5 Hz, 1H), 7.62 (d, J=10.8, 2.2 Hz, 1H) 7.50 (m, 1H), 7.16 (dd, J=10.8, 2.3 Hz, 2H), 6.94 (d, J=8.4, 1H), 6.89 (m, 1H), 4.12-4.16 (t, 4H); 3.66 (s, 3H), 3.60-3.45 (m, 3H); 3.40-3.52 (m, 3H), 2.88-2.94 (m, 4H); 2.54 (t, 2H); 2.21-2.36 (m, 4H).

APCI-MS m/z: 465, 467[MH⁺].

### 1-(3,4-Dichlorobenzyl)piperidin-4-amine (44a)

To a solution of tert-butylpiperidine-4-ylcarbamate (6.2 g, 31.0 mmol) in DMF (200 ml) was added 4-(bromomethyl)-1,2-dichlorobenzene (7.49 g, 30.96 mmol) and sodiumcarbonate (3.94 g, 37.2 mmol). The mixture was stirred at 60° C. for 3 h. Diluted with EtOAc and washed with water and brine. The organic layer was dried and concentrated in vacuo. The resulting crude product was dissolved in 20% CH₂Cl₂:TFA (200 ml) and stirred at room temperature for 16 h. The solvent was removed by evaporation and the residue was dissolved in water, basified by 2.0 M NaOH and extracted to dichloromethane, dried and concentrated in vacuo to give 1.40 g of the desired compound. Used without further purification.

### EXAMPLE 45

### {2-[((2S)-3-{[1-(3,4-dichlorobenzyl)piperidin-4-yl]amino}-2-hydroxy-2-methylpropyl)oxy]-4-fluorophenyl}acetic acid bis(trifluoroacetate) (salt)

The solution of 1-(3,4-dichlorobenzyl)piperidin-4-amine (54 mg, 0.210 mmol) and methyl (4-fluoro-2-{[(2S)-2-methyloxiran-2-yl]methoxy}phenyl)acetate (50 mg, 0.197 mmol) in methanol (3 ml) was stirred at 61° C. over night. The solvent was evaporated and to the residue was added MeOH:H₂O (1:1, 3 ml) and 2.0 M aqueous LiOH*H₂O (2.0 ml) and the resulting mixture was stirred at room temperature for 3 h, then diluted with dichloromethane and washed with conc. aqueous ammonium acetate. The water layer was washed with dichloromethane. The combined organic phases were dried, filtered and concentrated. Purified on HPLC to give 40 mg of the title compound.

¹H NMR (400 MHz, CD₃OD): δ 7.84 (s, 1H), 7.7 (d, J=9.5 Hz, 1H), 7.54 (dd, J=10.8, 2.2 Hz, 1H) 7.22 (t, J=7.5 Hz, 1H), 6.78 (dd, J=10.8, 2.3 Hz, 1H), 6.69 (td, J=8.4, 2.4 Hz, 1H), 4.33 (s, 2H), 3.97 (d, J=9.4 Hz, 1H), 3.91 (d, J=9.5 Hz, 1H), 3.64-3.43 (m, 5H), 3.38 (d, J=12.7 Hz, 1H), 3.16 (d, J=12.8 Hz, 1H), 3.11 (t, J=13.3 Hz, 2H), 2.43 (m, 2H), 2.05 (m, 2H), 1.44 (s, 3H).

APCI-MS m/z: 500.1 [MH⁺].

### EXAMPLE 46

### (2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid bis(trifluoroacetate) (salt)

(2S,5R)-1-(4-chlorobenzyl)-2,5-dimethylpiperidin-4-amine (46a, 35 mg, 140 82 mol) and methyl {4-fluoro-2-[(2S)-oxiran-2-ylmethoxy]phenyl}acetate (1a, 35 mg, 140 μmol) were dissolved in ethanol (3.0 ml) and the mixture was stirred for 36 h at 80° C. The sovlent was removed i. vac., and a mixture was purified by reverse phase HPLC (Kromasil column, 100-5-C₁₈, 250×20 mm) using acetonitrile (0.1% TFA) and H₂O (0.1% TFA). The following separation of the diastereomers by reverse phase HPLC (Purosphere column) using acetonitrile and H₂O (0.03% NH₃) afforded (2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetate (12 mg) and methyl (2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetate (6 mg, used in Example 48). Then (2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetate was dissolved in ethanol (1 ml), and aqueous 2 M NaOH (1 ml) was added. The mixture was stirred at 80° C. for 30 min. After cooling to room temperature the mixture was acidified with trifluoroacetic acid and concentrated under reduced pressure. The crude product was purified by reverse phase HPLC (Kromasil column, 100-5-C₁₈, 250×20 mm) using acetonitrile (0.1% TFA) and H₂O (0.1% TFA). After freeze-drying, 10 mg (11%) of the title compound 46 was obtained as a colourless powder.

¹H NMR (400 MHz, CD₃OD): δ 7.52 (m, 4H), 7.22 (dd, J=8.1, 6.7 Hz, 1H), 6.79 (dd, J=10.8, 2.3 Hz, 1H), 6.70 (td, J=8.4, 2.4 Hz, 1H), 4.74 (br.d, J=12.7 Hz, 1H), 4.13 (br.d, J=12.7 Hz, 1H), 3.96 (dd, J=20.4, 9.6 Hz, 2H), 3.60 (dd, J=20.5, 15.7 Hz, 2H), 3.49 (br.s, 1H), 3.37 (d, J=12.6 Hz, 1H), 3.35 (m, 1H), 3.23 (dd, J=12.7, 3.9 Hz, 1H), 3.14 (d, J=12.6 Hz, 1H), 2.80 (t, J=12.5 Hz, 1H), 2.37 (m, 2H), 2.11 (dd, J=25.7, 12.5 Hz, 1H), 1.63 (d, J=6.4 Hz, 3H), 1.44 (s, 3H), 1.06 (d, J=6.5 Hz, 3H).

APCI-MS m/z: 493 [MH⁺].

### (2S,5R)-1-(4-Chlorobenzyl)-2,5-dimethylpiperidin-4-amine (47a)

Prepared as described for 41a from (2S,5R)-1-(4-chlorobenzyl)-2,5-dimethylpiperidin-4-one oxime (34b, 167 mg, 630 μmol). Yield 71 mg (45%).

APCI-MS m/z: 253 [MH⁺].

### EXAMPLE 47

### (2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid bis(trifluoroacetate) (salt)

Methyl (2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetate (6 mg, 11 μmol, Example 46) was dissolved in ethanol (1 ml), and aqueous 2 M NaOH (1 ml) was added. The mixture was stirred at 80° C. for 30 min. After cooling to room temperature the mixture was acidified with trifluoroacetic acid and concentrated under reduced pressure. The crude product was purified by reverse phase HPLC (Kromasil column, 100-5-C₁₈, 250×20 mm) using acetonitrile (0.1% TFA) and H₂O (0.1% TFA). After freeze-drying, 8 mg (6%) of the title compound 47 was obtained as a colourless powder.

¹H NMR (400 MHz, CD₃OD) δ 7.52 (dd, J=12.4, 8.8 Hz, 4H), 7.22 (dd, J=8.2, 6.8 Hz, 1H), 6.78 (dd, J=10.8, 2.3 Hz, 1H), 6.69 (td, J=8.4, 2.4 Hz, 1H), 4.28 (br.s, 1H), 4.00 (d, J=9.6 Hz, 1H), 3.91 (d, J=9.9 Hz, 1H), 3.90 (br.s, 1H), 3.75 (br.s, 1H), 3.60 (dd, J=21.1, 15.9 Hz, 2H), 3.41 (d, J=12.7 Hz, 1H), 3.19 (m, 2H), 2.60 (br.s, 1H), 2.54 (m, 1H), 2.14 (m, 1H), 1.56 (d, J=6.7 Hz, 3H), 1.44 (s, 3H), 1.13 (d, J=7.3 Hz, 3H).

APCI-MS m/z: 493 [MH⁺].

### EXAMPLE 48

### (2-{[(2S)-3-{[1-(4-chlorobenzyl)-3,3-dimethylpiperidin-4-yl]amino}-2-hydroxy-2-methylpropyl]oxy}-4-fluorophenyl)acetic acid bis(trifluoroacetate) (salt)

To a solution of 1-(4-chlorobenzyl)-3,3-dimethylpiperidine-4-one (48a, 30 mg, 0.117 mmol) in methanol (3 ml) was added methyl (4-fluoro-2-{[(2S)-2-methyloxiran-2-yl]methoxy}phenyl)acetate (30 mg, 0.117 mmol). After the solution was stirred at 60° C. overnight 2.0 M LiOH.H₂O (2 ml) and MeOH:H₂O (1:1, 4 ml) was added and stirring was continued for additional 4 h. The solvent was evaporated and the resulting crude material was purified on RP-HPLC [Kromasil column, 100-5-C₁₈, acetonitrile (0.1% TFA) and H₂O (0.1% TFA)]. 7 mg of the title compound was obtained.

¹H NMR (400 MHz, CD₃OD) δ 7.48 (s, 4H), 7.22 (t, J=7.5 Hz, 1H), 6.78 (m, 1H), 6.69 (td, J=8.4, 2.4 Hz, 1H), 4.22 (m, 2H), 4.0 (t, 1H), 3.97 (t, 1H), 3.60 (s, 2H), 3.52 (m, 2H), 3.36 (d, J=12.8 Hz, 1H), 3.15 (m, 2H), 2.94 (m, 1H), 2.81 (d, 7.95, 1H), 2.12 (m, 1H), 1.43 (s, 3H), 1.25 (s, 6H).

APCI-MS m/z: 494.02 [MH⁺].

### 1-(4-Chlorobenzyl)-3,3-dimethylpiperidine-4-one (48a)

To a stirred solution of 1-(4-chlorobenzyl)-3,3-dimethylpiperidine-4-one in methanol (4 ml) was added ammonium acetate (214 mg, 2.78 mmol) and allowed to stand at 30° C. for 18h. The resulting mixture was cooled to 0° C. and sodium cyanoborohydride (175 mg, 2.78 mmol) was added. After 15 min the cooling was removed and the mixtured stirred for 18 h at room temperature. The mixture was then concentrated to dryness, and triturated with water and the water was acidified with conc. HCl(˜pH 3) and extracted to EtOAc. The organic phase was dried and concentrated to dryness to give 35 mg of the subtitle compound, which was used in Example 48 without further purification.

### 1-(4-Chlorobenzyl)-3,3-dimethylpiperidine-4-one (48b)

The mixture of 3,3-dimethyl-4-oxopiperidine-4-one (4c) (95 mg, 0.746 mmol) and potassium carbonate (516 mg) in DMF was stirred at room temperature for 1 h. 1-(bromomethyl)-4-chlorobenzene (154 mg, 0.746 mmol) was added and the mixture stirred for further 4 h at room temperature, diluted with EtOAc and washed with water. The organic phase was dried and concentrated to give 150 mg crude product, which was used without further purification.

### 3,3-Dimethyl-4-oxopiperidine-4-one (48c)

To an ice-bath cooled solution of tert-butyl 4-oxopiperidine-1-carboxylate (315 mg, 1.58 mmol) in THF (5 ml) was added iodomethane (144 μl) and sodium tert-butoxide (196 mg, 2.04 mmol). The mixture was allowed to attain room temperature and stirring was continued at room temperature over night. The solvent was then removed by evaporation in vacuo, the residue was taken up in dichloromethane and washed with water, brine, dried and the solvent was evaporated. To the residue TFA (20% in CH₂Cl₂) was added and the solution was stirred at room temperature for 6 h, thereafter concentrated in vacuo and the crude was used without further purification.

### EXAMPLE 49

### (2S)-1{[1-(4-chlorobenzyl)piperidin-4-yl]amino}-3-[2-(1H-tetrazol-5-yl)phenoxy]propan-2-ol bis(trifluoroacetate) (salt)

To the solution of 2-[((2S)-3-{[1-(4-chlorobenzyl)piperidin-4-yl]amino}-2-hydroxypropyl)oxy]benzonitrile (49a) (1.8 g, 4.5 mmol) in NMP (50 ml) under argon was added sodiumazide (877 mg, 13.5 mmol) and triethylamine hydrochloride (947 mg, 6.88 mmol). The mixture was warmed to 150° C., stirred for 4 h. After cooling to ambient temperature the mixture was diluted with water (150 ml) and extracted with EtOAc (3*200 ml) after pH was adjusted to 4 by 2 M HCl. The organic layer was extracted with 10% (w/w) aqueous NaOH. The alkaline extract was washed with diethylether, then pH was adjusted to 4, extracted with EtOAc, dried over Na₂SO₄, filtered and the solvent evaporated. Purification by RP-HPLC [Kromasil column, 100-5-C₁₈, acetonitrile (0.1% TFA) and H₂O (0.1% TFA)] gave the title compound (120 mg, 30%).

### 2-[((2S)-3-{[1-(4-chlorobenzyl)piperidin-4-yl]amino}-2-hydroxypropyl)oxy]benzonitrile (49a)

The solution of 2-[(2S)-oxiran-2-ylmethoxy]benzonitile (49b) (1.42 g, 8.0 mmol) and 1-(4-chlorobenzyl)piperidine-4-amine (2.0 g, 8.9 mmol) in ethanol was stirred at 80° C. for to 4 hrs. After cooling the solvent was evaporated in vacuo. The crude material was taken up with dichloromethane, washed with water, dried over Na₂SO₄ and concentrated in vacuo to give the crude product. Flash chromatography on silica gel (4:1 EtOAc:MeOH) afforded 1.9 g of the title compound 49a (59%).

### 2-[(2S)-oxirane-2-ylmethoxy]benzonitrile (49b)

A mixture of (2S)-2-oxiran-2-ylmethyl 3-nitrobenzenesulfonate (90.8 g, 41.97 mmol), 2-hydroxybenzonitrile (5 g, 41.97 mmol) and Cs₂CO₃ (27.35 g, 83.94 mmol) in DMF (300 ml) was stirred at room temperature for 3 h. The reaction mixture was partitioned between EtOAc and H₂O. The organic layer was dried over Na₂SO₄, filtered and concentrated in vacuo. The resulting crude product (6g) was used without further purification.

### EXAMPLE 50

### (2S)-1-{[1-(4-chlorobenzyl)piperidin-4-yl]amino}-2-methyl-3-[2-(1H-tetrazol-5-yl)phenoxy]propan-2-ol

The title compoud was prepared according to the protocol described for Example 49 from 2-[((2S)-3-{[1-(4-chlorobenzyl)piperidin-4-yl]amino}-2-hydroxy-2-methylpropyl)oxy]benzonitrile (50a) and sodium azide (197 mg, 3.02 mmol) and triethylammonium hydrochloride (211 mg, 1.82 mmol) in NMP (4 ml) and 160 mg (35%) were obtained as HCl salt.

### 2-[((2S)-3-{[1-(4-chlorobenzyl)piperidin-4-yl]amino}-2-hydroxy-2-methylpropyl)oxy]benzonitrile (50a)

The subtitle compound was prepared according to the protocol described for (49a) starting from 2-{[(2S)-2-methoxyoxiran-2-yl]methoxy}benzonitrile (50b) and 1-(4-chlorobenzyl)piperidine-4-amine (227 mg, 1.01 mmol). The resulting residue was used to the next step without further purification.

### 2-[(2S)-Methyloxirane-2-ylmethoxy]benzonitrile (50b)

The subtitle compound was prepared according to the protocol described for (49b) starting from [(2S)-2-methyloxiran-2-yl]methyl 3-nitrobenzenesulfonate (276 mg, 1.06 mmol), 2-hydroxybenzonitrile (120 mg, 1.01 mmol) and Cs₂CO₃ (700 mg, 2.14 mmol) in DMF (4 ml). The material was used without further purification.

### EXAMPLE 51

### (2S)-1-{[1-(4-chlorobenzyl)piperidin-4-yl]amino}-3[2-(1H-tetrazol-5-ylmethyl)phenoxy]propan-2-ol bis(trifluoroacetate) (salt)

1-(4-Chlorobenzyl)piperidin-4-amine (24 mg, 105 mmol) and 5-{2-[(2S)-oxiran-2-ylmethoxy]benzyl}-2-trityl-2H-tetrazole (51a, 50 mg, 105 μmol) were dissolved in ethanol (3.0 ml) and the mixture was stirred overnight at 80° C. Then the solvent was revoved i.vac, the residue dissolved in dichloromethane (10 ml). Trifluoroacetic acid (2 ml) was added, and the mixture was stirred at room temperature for 2 h. The mixture was concentrated under reduced pressure. The crude product was isolated by reversed phase HPLC (Kromasil column, 100-5-C₁₈, 250×20 mm) using acetonitrile (0.1% TFA) and H₂O (0.1% TFA). After freeze-drying, 7 mg (10%) of the title compound 1 was obtained as a colourless powder.

¹H NMR (400 MHz, CD₃OD) δ 7.54 (dd, J=20.1, 8.5 Hz, 4H), 7.36 (d, J=7.3 Hz, 2H), 7.32 (dd, J=15.8, 1.4 Hz, 2H), 6.99 (m, 2H), 4.38 (s, 2H), 4.32 (d, J=2.8 Hz, 2H), 4.20 (m, 1H), 4.06 (dd, J=9.8, 4.2 Hz, 1H), 3.97 (dd, J=9.8, 5.8 Hz, 1H), 3.66 (br.d, J=12.5 Hz, 2H), 3.55 (m, 1H), 3.22 (m, 3H), 3.10 (dd, J=12.4, 9.8 Hz, 1H), 2.46 (m, 2H), 2.10 (m, 2H).

APCI-MS m/z: 457 [MH⁺].

### 5-{2-[(2S)-Oxiran-2-ylmethoxy]benzyl}-2-trityl-2H-tetrazole (51a)

The subtitled compound was prepared according to the protocol described for 1a starting from 2-[(2-trityl-2H-tetrazol-5-yl)methyl]phenol (51b, 50 mg, 120 mmol) and (25)-oxiran-2-ylmethyl 3-nitrobenzenesulfonate (31 mg, 120 μmol). Yield: 50 mg (88%) as colourless oil.

¹H NMR (400 MHz, CDCl₃) δ 7.35-7.20 (m, 9H), 7.16 (dd, J=15.6, 1.5 Hz, 1H), 7.11-7.04 (m, 6H), 6.86 (t, J=7.5 Hz, 1H), 6.80 (d, J=8.2 Hz, 1H), 4.28 (d, J=1.4 Hz, 2H), 4.10 (dd, J=11.6, 3.4 Hz, 1H), 3.92 (dd, J=11.0, 5.2 Hz, 1H), 3.16 (m, 1H), 2.72 (t, J=4.5 Hz, 1H), 2.66 (dd, J=5.0, 2.7 Hz, 1H).

### 2-[(2-Trityl-2H-tetrazol-5-yl)methyl]phenol (51b)

A stirred solution of 5-(2-methoxybenzyl)-1H-tetrazole (51c, 478 mg, 2.3 mmol) in dichloromethane (10 ml) was cooled to 0° C. Boron tribromide (1 M in solution in dichloromethane, 5.0 ml, 5.0 mmol) was added dropwise. The stirring was continued at room temperature for 3 h. Then the mixture was quenched with methanol (2 ml), and extracted with water (2×10 ml), and 1 M NaOH (2×10 ml). The combined aqueous extracts were acidified with HCl (2 M) to pH 1, and evaporated to dryness. The solid residue was extracted with acetone. Evaporation of acetone yielded the white residue of 2-(1H-tetrazol-5-ylmethyl)phenol, which was dissolved in water (20 ml). To the solution were added aqueous NaOH (2M, 0.5 ml, 100 μmol), tetrabutylammonium brimide (16 mg, 50 μmol), and dichloromethane (20 ml). A solution of 1,1′,1″-(chloromethanetriyptribenzene (278 mg, 100 mmol) in dichloromethane (10 ml) was added dropwise with vigorous stirring. The stirring was continued for 2 h at room temperature, then the layers were separated. The organic layer was dried with sodium sulfate, and the solvent was removed in vacuo. Purification by flash chromatography on silica gel (ethyl acetate/n-heptane, 1:4) afforded white solid, 150 mg (36%).

¹H NMR (400 MHz, CDCl₃) δ 7.65 (br.s, 1H), 7.41-7.30 (m, 9H), 7.22-7.16 (m, 2H), 7.09 (m, 6H), 6.97 (d, J=8.3 Hz, 1H), 6.88 (t, J=7.4 Hz, 1H), 4.25 (s, 2H).

### 5-(2-Methoxybenzyl)-1H-tetrazole (51c)

A mixture of (2-methoxyphenyl)acetonitrile (1.47 g, 10 mmol), sodium azide (1.95 g, 30 mmol), and triethylamine hydrochloride (2.06 g, 15 mmol) in 1-methylpyrrolidine-2-one (10 ml) was stirred at 130° C. for 16 h. Then the mixture was coled to room temperature, and poured into water (100 ml). The pH was adjusted to 1 with conc. HCl, and the mixture was extracted with ethyl acetate (3×50 ml). The combined organic fractions were washed with water, and dried with sodium sulfate. Evaporation of the solvent yielded brown crystals, 1.97 g (quantitative).

¹1-1 NMR. (400 MHz, CDCl₃) δ 7.25 (m, 2H), 6.91 (m, 2H), 4.31 (s, 2H), 3.87 (s, 3H).

APCI-MS m/z: 191 [MH⁺].

### EXAMPLE 52

### (2S)-1-{[1-(4-Chlorobenzyl)piperidin-4-yl]amino}-2-methyl-3-[2-(1H-tetrazol-5-ylmethyl)phenoxy]propan-2-ol bis(trifluoroacetate) (salt)

A mixture of (2-{[(2S)-3-{[1-(4-chlorobenzyl)piperidin-4-yl]amino}-2-hydroxy-2-methylpropyl]oxy}phenyl)acetonitrile (52a, 178 mg, 0.416 mmol), NaN₃ (270 mg, 4.16 mmol) and NH₄Cl (223 mg, 4.16 mmol) in DMF (2 ml) was stirred at 80° C. for 24 h. Another portion of NaN₃ (270 mg) and NH₄Cl (223 mg) were added and stirred at 80° C. for 24 h, cooled to room temperature and this was subjected to HPLC (10-60% acetonitrile in water, 0.1% TFA) to give the title compound (55 mg).

¹H-NMR (CDCl₃, 400 MHz): δ 7.52 (s, 4H); 7.40 (m, 1H); 7.32 (m, 1H); 7.00 (m, 2H); 4.38 (s, 4H); 3.91 (s, 2H); 3.66 (m, 2H); 3.50 (m, 1H); 3.30 (d, 1H); 3.18 m, 2H); 3.09 (d, J=12.7 Hz, 1H); 2.49 (m, 2H); 2.09 (m, 2H); 1.33 (s, 3H).

APCI-MS: m/z 471 (MH⁺).

(2-{[(2S)-3-{[1-(4-Chlorobenzyl)piperidin-4-yl]amino}-2-hydroxy-2-methylpropyl]oxy}phenyl)acetonitrile (52a)

A mixture of [(2S)-2-methyloxiran-2-yl]methyl 3-nitrobenzenesulfonate (232 mg, 0.845 mmol), (2-{[(2S)-2-methyloxiran-2-yl]methoxy}phenyl)acetonitrile (52b, 113 mg, 0.848 mmol) and Cs₂CO₃ (332 mg, 1.02 mmol) in DMF (3 ml) was stirred at room temperature over night, partitioned between ethyl acetate and water. The organic layer was dried over Na₂SO₄, filtered and the filtrate was concentrated in vacuo. The residue was taken into methanol (3 ml), 1-(4-chlorobenzyl)piperidin-4-amine (190 mg, 0.848 mmol) was added and the reaction mixture was stirred at 80° C. for 7 h. The volatiles were removed in vacuo and the residue was purified by flash chromatography on silica gel (0-2% methanol in dichloromethane, 0.2% NH₄OH) to give subtitle compound (182 mg).

¹H-NMR (DMSO-d₆, 400 MHz): δ 7.37-7.26 (m, 6H); 7.01 (d, J=8.3 Hz, 1H); 6.95 (m, 1H); 4.52 (br.s, 1H); 3.90 (d, J=8.7 Hz, 1H); 3.85 (s, 2H); 3.75 (d, J=8.7 Hz, 1H); 3.40 (s, 2H); 2.62 (m, 4H); 2.35 (m, 1H); 1.91 (m, 2H); 1.78 (m, 2H); 1.10 (m, 5H).

APCI-MS: m/z 428 (MH⁺).

(2-{[(2S)-2-Methyloxiran-2-yl]methoxy}phenyl)acetonitrile (52b) 2-(Hydroxymethyl)phenol (620 mg, 5 mmol) was treated with NaCN (245 mg, 5 mmol) in DMF at 120° C. for 5.5 h, cooled to room temperature, partitioned between ethyl acetate and water. The organic layer was washed with aqueous Saturated NH₄Cl, dried over Na₂SO₄, filtered and the filtrate was concentrated in vacuo. The residue was purified by flash chromatography on silica gel (0-30% ethyl acetate in petrolium spirit 40-60) to give subtitle compound (120 mg). (Bioorg. Med. Chem. 2005, 13, 1989)

¹H-NMR (DMSO-d₆, 400 MHz): δ 10.18 (br.s, 1H); 7.24 (d, J=7.4 Hz, 1H); 7.15 (m, 1H); 6.86 (d, J=7.4 Hz, 1H); 6.80 (m, 1H); 3.78 (s, 2H). is APCI-MS: m/z 134 (MH⁺).

### EXAMPLE 53

### (2S)-1-{[1-(4-Chlorobenzyl)piperidin-4-yl]amino}-3-[5-fluoro-2-(1H-tetrazol-5-ylmethyl)phenoxy]-2-methylpropan-2-ol bis(trifluoroacetate)

A suspension of (2-{[(2S)-3-{[1-(4-chlorobenzyl)piperidin-4-yl]amino}-2-hydroxy-2-methylpropyl]oxy}-4-fluorophenyl)acetonitrile ((53a, 346 mg, 0.77 mmol), NaN₃ (650 mg, 10 mmol) and NH₄Cl (536 mg, 10 mmol) in DMF (5 ml) was stirred at 80° C. for 48 h, cooled to room temperature and subjected to HPLC (10-60% acetonitrile in water, 0.1% TFA) to give the title compound (140 mg).

¹H-NMR (CD₃OD, 400 MHz): δ 7.51 (s, 4H); 7.39 (dd, J=6.5, 8.3 Hz, 1H); 6.77 (m, 2H); 4.31 (s, 4H); 3.90 (m, 2H); 3.60 (br.d, J=12.2 Hz, 2H); 3.49 (m, 1H); 3.23 (d, J=12.7 Hz, 1H); 3.10 (m, 2H); 3.02 (d, J=12.7 Hz, 1H); 2.41 (br.d, J=13.3 Hz, 2H); 2.05 (m, 2H); 1.35 (s, 3H).

APCI-MS: m/z 489 (MH⁺).

### (2-{[(2S)-3-{[1-(4-Chlorobenzyl)piperidin-4-yl]amino}-2-hydroxy-2-methylpropyl]oxy}-4-fluorophenyl)acetonitrile (53a)

A mixture of [(2S)-2-methyloxiran-2-yl]methyl 3-nitrobenzenesulfonate (317 mg, 1.16 mmol), (4-fluoro-2-hydroxyphenyl)acetonitrile (53b, 176 mg, 1.16 mmol) and Cs₂CO₃ (453 mg, 1.39 mmol) in DMF (3 ml) was stirred at room temperature over night. The reaction mixture was partitioned between ethyl acetate and water. The organic layer was dried over Na₂SO₄, filtered and the filtrate was concentrated in vacuo. The residue was dissolved in methanol (2 ml), 1-(4-chlorobenzyl)piperidin-4-amine 260 mg, 1.16 mmol) was added and stirred at 80° C. for 7 h, cooled to room temperature and the volatiles were removed in vacuo. The residue was purified by flash chromatography on silica gel (0-2% methanol in dichloromethane, 0.2% NH₄OH) to give subtitle compound (350 mg).

¹H-NMR (CD₃OD, 400 MHz): δ 7.26 (m, 5H); 6.68 (m, 2H); 3.85 (s, 2H); 3.62 (s, 2H); 3.45 (s, 2H); 3.00 (d, J=12.6 Hz, 1H); 2,80 (br.d, J=11.1 Hz, 2H); 2.59 (d, J=12.6 Hz, 1H); 2.44 (m, 1H); 2.00 (br.d, J=11.3 Hz, 2H); 1.88 (m, 2H); 1.37 (m, 2H); 1.35 (s, 3H).

APCI-MS: m/z 446 (MH⁺).

### (4-Fluoro-2-hydroxyphenyl)acetonitrile (53b)

To a cold (ice-water bath) solution of LiAlH₄ in THF (10 ml, 10 mmol) was added a solution of 4-fluoro-2-hydroxybenzoic acid (624 mg, 4 mmol) in THF (5 ml) slowly. After addition was completed ice bath was removed and the reaction mixture was stirred at room temperature for about 2½ day, cooled to 0° C., dil. aqueous HCl (20 ml) was added. carefully. The ice bath was removed and after 40 min at room temperature the reaction mixture, was extracted with ethyl acetate. The organic layer was washed with water and aqueous NaHCO₃ respectively, dried over Na₂SO₄, filtered and the filtrate was concentrated in vacuo. The residue was dissolved in DMF (3 ml), NaCN (230 mg, 4.69 mmol) was added and the reaction mixture was stirred at 120° C. for 4.5 h, cooled to room temperature and extracted with ethyl acetate. The organic layer was washed with aqueous

NH₄Cl, dried over Na₂SO₄, filtered and the filtrate was concentrated in vacuo. The residue was purified by silica gel flash chromatography (0-20% ethyl acetate in petroleum spirit 40-60) to give subtitle compound (180 mg).

¹H-NMR. (DMSO-d₆, 400 MHz): δ 10.60 (br.s, 1H); 7.26 (t, J=8.0 Hz, 1H); 6.64 (m, 2H); 3.68 (s, 2H).

### Human CCR1 Binding Assay Membranes

HEK293 cells, from ECACC, stably expressing recombinant human CCR1 (HEK-CCR1) were used to prepare cell membranes containing CCR1. The membranes were stored at −70° C. The concentration of membranes of each batch was adjusted to 10% specific binding of 33 μM [¹²⁵I] MIP-1α.

### Binding Assay

100 μL of HEK-CCR1 membranes diluted in assay buffer pH 7.4 ((137 mM NaCl (Merck, Cat No 1.06404), 5.7 mM Glucose (Sigma, Cat No G5400), 2.7 mM KCl (Sigma, Cat No P-9333), 0.36 mM NaH₂PO₄×H₂O (Merck, Cat No 1.06346), 10 mM HEPES (Sigma, Cat No H3375), 0.1% (w/v) Gelatine (Sigma, Cat No G2625)) with the addition of 17500 units/L Bacitracin (Sigma, Cat No B 1025) were added to each well of the 96 well filter plate (0.45 μm opaque Millipore cat no MHVB N4550). 12 μl of compound in assay buffer, containing 10% DMSO, was added to give final compound concentrations of 1×10^{-5.5}−1×10^{-9.5} M. 12 μl cold human recombinant MIP-1α (270-LD-050, R&D Systems, Oxford, UK), 10 nM final concentration in assay buffer supplemented with 10% DMSO, was included in certain wells (without compound) as non-specific binding control (NSB). 12 μl assay buffer with 10% DMSO was added to certain wells (without compound) to detect maximal binding (B0).

12 μl [¹²⁵I] MIP-1α, diluted in assay buffer to a final concentration in the wells of 33 μM, was added to all wells. The plates with lid were then incubated for 1.5 hrs at room temperature. After incubation the wells were emptied by vacuum filtration (MultiScreen Resist Vacuum Manifold system, Millipore) and washed once with 200 μl assay buffer. After the wash, all wells received an addition of 50 μl of scintillation fluid (OptiPhase “Supermix”, Wallac Oy, Turko, Finland). Bound [¹²⁵I] MIP-1α was measured using a Wallac Trilux 1450 MicroBeta counter. Window settings: Low 5-High 1020, 1-minute counting/well.

Calculation of percent displacement and IC₅₀

The following equation was used to calculate percent displacement.

Percent displacement=1−((cpm test−cpm NSB)/(cpm B0−cpm NSB)) where:

cpm test=average cpm in wells with membranes and compound and [¹²⁵I] MIP-1a;

NSB=average cpm in the wells with membranes and MIP-1α and [¹²⁵I] MIP-1α (non-specific binding);

B0=average cpm in wells with membranes and assay buffer and [¹²⁵I] MIP-1a (maximum binding).

The molar concentration of compound producing 50% displacement (IC₅₀) was derived using the Excel-based program XLfit (version 2.0.9) to fit data to a 4-parameter logistics function.

An IC₅₀ value of less than 1 μM was measured for each of the example compounds.

1. A compound of formula 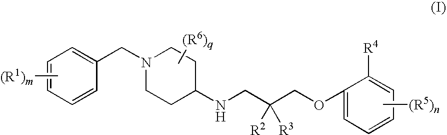
wherein
m is 0, 1 or 2;
n is 0, 1 or 2;
q is 0, 1, 2, 3 or 4;
each R^{I} independently represents halogen, C₁-C₆ alkyl, C₁-C₆ alkoxy, OCF₃ or CF₃;
R² represents a hydrogen atom, C₁-C₆ alkyl or C₃-C₇ cycloalkyl;
R³ represents a hydrogen atom or hydroxyl group;
R⁴ represents —C₁-C₆ alkyl-COOH, —C₁-C₆ alkoxy-COOH, —C₃-C₄ cycloalkyl-COOH, or —(CH₂)ₜ-tetrazole;
t is 0, 1 or 2;
each R⁵ independently represents halogen, C₁-C₆ alkyl, OCF₃ or CF₃;
each R⁶ represents C₁-C₆ alkyl;
or a pharmaceutically acceptable salt thereof.

2. A compound according to claim 1, wherein m is 1 and R¹ represents halogen.

3. A compound according to claim 2, wherein R¹ represents chlorine in the 4-position of the benzene ring relative to the carbon atom to which the CH₂ linking group is attached.

4. A compound according to any preceding claim, wherein R⁴ represents —(CH₂)ₚ—COOH, where p is 1, 2, 3, or 4.

5. A compound according to any preceding claim, wherein R⁴ represents —CH₂—COOH, —(CH₂)₂—COOH, -methoxy-COOH, -ethoxy-COOH, -cyclopropyl-COOH, -tetrazole, or -methyl-tetrazole.

6. A compound according to any preceding claim, wherein each R⁵ represents halogen.

7. A compound according to any preceding claim, wherein n is 0 or 1, and R⁵ represents halogen.

8. A compound according to any preceding claim, wherein q is 0, 1 or 2.

9. A compound according to any preceding claim, wherein R₂ is methyl.

10. A compound according to any preceding claim, wherein R₃ is hydroxyl.

11. A compound
{2-[((2S)-3-{[1-(4-chlorobenzyl)piperidin-4-yl]amino}-2-hydroxy-2-methylpropyl)oxy]-4-fluorophenyl}acetic acid,
{4-chloro-2-[((2S)-3-{[1-(4-chlorobenzyl)piperidin-4-yl]amino}-2-hydroxy-2-methylpropyl)oxy]phenyl}acetic acid,
{2-[((2S)-3-{[1-(4-chlorobenzyl)piperidin-4-yl]amino}-2-hydroxy-2-methylpropyl)oxy]phenyl}acetic acid,
3-{2-[((2S)-3-{[1-(4-chlorobenzyl)piperidin-4-yl]amino}-2-hydroxypropyl)oxy]-4-fluorophenyl}propanoic acid,
{5-chloro-2-[((2S)-3-{[1-(4-chlorobenzyl)piperidin-4-yl]amino}-2-hydroxy-2-methylpropyl)oxy]phenyl}acetic acid,
3-{2-[((2S)-3-{[1-(4-chlorobenzyl)piperidin-4-yl]amino}-2-hydroxy-2-methylpropyl)oxy]-4-fluorophenyl}propanoic acid,
3-[2-(3-{[1-(4-chlorobenzyl)piperidin-4-yl]amino}propoxy)phenyl]propanoic acid,
3-{2-[((2S)-3-{[1-(4-chlorobenzyl)piperidin-4-yl]amino}-2-hydroxy-2-methylpropyl)oxy]phenyl}propanoic acid,
{5-chloro-2-[((2S)-3-{[1-(4-chlorobenzyl)piperidin-4-yl]amino}-2-hydroxypropyl)oxy]phenyl}acetic acid,
[2-(3-{[1-(4-chlorobenzyl)piperidin-4-yl]amino}propoxy)phenyl]acetic acid,
3-{5-chloro-2-[((2S)-3-{[1-(4-chlorobenzyl)piperidin-4-yl]amino}-2-hydroxy-2methylpropyl)oxy]phenyl}propanoic acid,
[5-chloro-2-(3-{[1-(4-chlorobenzyl)piperidin-4-yl]amino}propoxy)phenyl]acetic acid,
3-{2-[((2S)-3-{[1-(4-chlorobenzyl)piperidin-4-yl]amino}-2-hydroxy-2-methylpropyl)oxy]-5-fluorophenyl}propanoic acid,
(2-{[(2S)-3-{[1-(4-chlorobenzyl)piperidin-4-yl]amino}-2-hydroxypropyl]oxy}-4-fluorophenyl)acetic acid,
3-[2-(3-{[1-(4-chlorobenzyl)piperidin-4-yl]amino}propoxy)-4-fluorophenyl]propanoic acid,
[2-{[(2S)-3-{[1-(4-chlorobenzyl)piperidin-4-yl]amino}-2-hydroxy-2-methylpropyl]oxy}-4-(trifluoromethyl)phenyl]acetic acid,
(2-{[(2S)-3-{[1-(4-fluorobenzyl)piperidin-4-yl]amino}-2-hydroxypropyl]oxy}phenyl)acetic acid,
(2-{[(2S)-3-{[1-(4-chloro-3-(trifluoromethyl)benzyl]piperidin-4-yl}amino)-2-hydroxypropyl]oxy}phenyl)acetic acid,
(2-{[(2S)-3-{[1-(3,4-difluorobenzyl)piperidin-4-yl]amino}-2-hydroxypropyl]oxy}phenyl)acetic acid,
(2-{[(2S)-3-{[1-(2,5-dimethylbenzyl)piperidin-4-yl]amino}-2-hydroxypropyl]oxy}phenyl)acetic acid,
(2-{[(2S)-3-{[1-(2-chloro-4-fluorobenzyl)piperidin-4-yl]amino}-2-hydroxypropyl]oxy}phenyl)acetic acid,
(2-{[(2S)-3-{[1-(3-chloro-4-fluorobenzyl)piperidin-4-yl]amino}-2-hydroxypropyl]oxy}phenyl)acetic acid,
(2-{[(2S)-3-{[1-(3,5-dimethylbenzyl)piperidin-4-yl]amino}-2-hydroxypropyl]oxy}phenyl)acetic acid,
(2-{[(2S)-3-{[1-(4-chloro-2-fluorobenzyl)piperidin-4-yl]amino}-2-hydroxypropyl]oxy}phenyl)acetic acid,
(2-{[(2S)-3-{[1-(4-bromobenzyl)piperidin-4-yl]amino}-2-hydroxypropyl]oxy}phenyl)acetic acid,
(2-{[(2S)-3-{[1-(4-chlorobenzyl)piperidin-4-yl]amino}-2-hydroxy-2-methylpropyl]oxy}-3,4-difluorophenyl)acetic acid,
2-(2-{[(2S)-3-{[1-(4-chlorobenzyl)piperidin-4-yl]amino}-2-hydroxypropyl]oxy}phenyl)propanoic acid,
{2-[2-({[1-(4-chlorobenzyl)piperidin-4-yl]amino}methyl)-2-hydroxybutoxy]phenyl}acetic acid,
(2-{[2S)-3-{[1-(4-chlorobenzyl)-2,5-dimethylpiperidin-4-yl]amino}-2-hydroxy-2-methylpropyl]oxy}phenyl)acetic acid,
(2-{[(2S)-3-{[1-(4-chloro-2-methylbenzyl)piperidin-4-yl]amino}-2-hydroxypropyl]oxy}phenyl)acetic acid,
2-(2-{[(2S)-3-{[1-(4-chlorobenzyl)piperidin-4-yl]amino}-2-hydroxy-2-methylpropyl]oxy}phenyl)propanoic acid,
1-(2-{[(2S)-3-{[1-(4-chlorobenzyl)piperidin-4-yl]amino}-2-hydroxypropyl]oxy}phenyl)cyclopropanecarboxylic acid,
1-(2-{[(2S)-3-{[1-(4-chlorobenzyl)piperidin-4-yl]amino}-2-hydroxy-2-methylpropyl]oxy}phenyl)cyclopropanecarboxylic acid,
(2-{[(2S)-3-{[(2R,5S)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[(3S,4R)-1-(4-chlorobenzyl)-3-methylpiperidin-4-yl]amino}-2-hydroxy-2-methylpropyl]oxy}-4-fluorophenyl)acetic acid,
(2-{[(2S)-3-{[(3R,4R)-1-(4-chlorobenzyl)-3-methylpiperidin-4-yl]amino}-2-hydroxy-2-methylpropyl]oxy}-4-fluorophenyl)acetic acid,
2-(2-{[(2S)-3-{[1-(4-chlorobenzyl)piperidin-4-yl]amino}-2-hydroxy-2-methylpropyl]oxy}-4-fluorophenyl)propanoic acid,
2-[2-(3-{[1-(4-chlorobenzyl)piperidin-4-yl]amino}propoxy)phenoxy]propanoic acid,
[2-(3-{[1-(4-chlorobenzyl)piperidin-4-yl]amino}propoxy)phenoxy]acetic acid,
(2-{[(2S)-3-{[1-(4-chlorobenzyl)-2,2,6,6-tetramethylpiperidin-4-yl]amino}-2-hydroxy-2-methylpropyl]oxy}-4-fluorophenyl)acetic acid,
(2-{[(2S)-3-{[(2R,4S,5S)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[(2R,4R,5S)-1-(4-chlorobenzyl)-2,5-dimethylpiperidin-4-yl]amino}-2-hydroxy-2-methylpropyl]oxy}-4-fluorophenyl)acetic acid,
3-[2-(3-{[(2R,4S,5S)-1-(4-chlorobenzyl)-2,5-dimethylpiperidin-4-yl]amino}propoxy)phenyl]propanoic acid,
3-[2-(3-{[1-(3,4-dichlorobenzyl)piperidin-4-yl]amino}propoxy)phenyl]propanoic acid,
(2-{[(2S)-3-{[1-(3,4-dichlorobenzyl)piperidin-4-yl]amino}-2-hydroxy-2-methylpropyl]oxy}-4-fluorophenyl)acetic acid,
(2-{[(2S)-3-{[(2S,4R,5R)-1-(4-chlorobenzyl)-2,5-dimethylpiperidin-4-yl]amino}-2-hydroxy-2-methylpropyl]oxy}-4-fluorophenyl)acetic acid,
(2-{[(2S)-3-{[(2S,4S,5R)-1-(4-chlorobenzyl)-2,5-dimethylpiperidin-4-yl]amino}-2-hydroxy-2-methylpropyl]oxy}-4-fluorophenyl)acetic acid,
{2-[(2S)-3-{[1-(4-chlorobenzyl)-3,3-dimethylpiperidin-4-yl]amino}-2-hydroxy-2-methylpropyl]oxy}-4-fluorophenyl)acetic acid,
(2S)-1-{[1-(4-chlorobenzyl)piperidin-4-yl]amino}-3-[2-(1H-tetrazol-5-yl)phenoxy]propan-2-ol,
(2R)-1-{[1-(4-chlorobenzyl)piperidin-4-yl]amino}-2-methyl-3[2-(1H-tetrazol-5-yl)phenoxy]propan-2-ol,
(2S)-1-{[1-(4-chlorobenzyl)piperidin-4-yl]amino}-3-[2-(1H-tetrazol-5-ylmethyl)phenoxy]propan-2-ol,
(2S)-1-{[1-(4-chlorobenzyl)piperidin-4-yl]amino}-2-methyl-3[2-(1H-tetrazol-5-ylmethyl)phenoxy]propan-2-ol, or
(2S)-1-{[1-(4-chlorobenzyl)piperidin-4-yl]amino}-3-[5-fluoro-2-(1H-tetrazol-5-ylmethyl)phenoxy]-2-methylpropan-2-ol,
or a pharmaceutically acceptable salt thereof.

12. A process for preparing a compound of formula (I) or a pharmaceutically acceptable salt thereof as defined in claim 1 which process comprises
(a) where R³ is a hydroxyl group, reacting a compound of formula 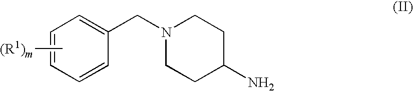
wherein m and R¹ are as defined in formula (I), with a compound of formula 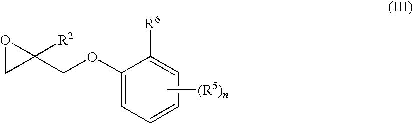
wherein R², R⁵ and n are as defined in formula (I), and R⁶ is R⁴ as defined formula (I) or a protected derivative thereof; or
(b) where R³ is a hydroxyl group, reacting a compound of formula 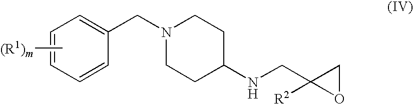
wherein m, R¹ and R² are as defined in formula (I), with a compound of formula 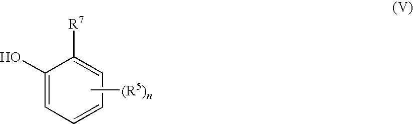
wherein R⁵ and n are as defined in formula (I), and R⁶ is R⁴ as defined formula (I) or a protected derivative thereof; or
(c) reacting a compound of formula 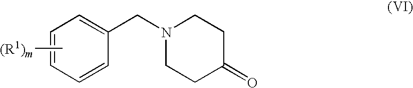
wherein m and R¹ are as defined in formula (I), with a compound of formula 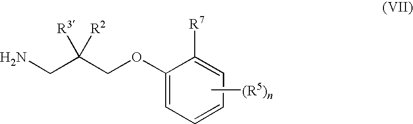
wherein R², R⁵ and n are as defined in formula (I), R³′ is Rias defined in formula (I) or —O—P where P is a suitable protecting group, and R⁶ is R⁴ as defined formula (I) or a protected derivative thereof;
(d) reacting a compound of formula 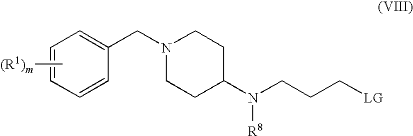
wherein m and R¹ are as defined in formula (I), R⁷ is H or a suitable protecting group, and LG is a suitable leaving group, with a compound of formula 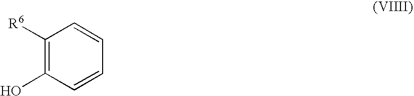
wherein R⁶ is R⁴ as defined in formula (I) or a protected derivative thereof;
to and optionally after (a), (b), (c) or (d) removing any protecting groups and/or forming a pharmaceutically acceptable salt of the compound of formula (I).

13. A pharmaceutical composition comprising a compound of formula (I), or a pharmaceutically acceptable salt thereof, as claimed in any one of claims 1 to 11 in association with a pharmaceutically acceptable adjuvant, diluent or carrier.

14. A process for the preparation of a pharmaceutical composition as claimed in claim 13 which comprises mixing a compound of formula (I), or a pharmaceutically acceptable salt thereof, as claimed in any one of claims 1 to 11 with a pharmaceutically acceptable adjuvant, diluent or carrier.

15. A compound of formula (I), or a pharmaceutically-acceptable salt thereof, as claimed in any one of claims 1 to 11 for use in therapy.

16. Use of a compound as claimed in any one of claims 1 to 11 or a pharmaceutically acceptable salt thereof, in the manufacture of a medicament for treating a respiratory disease.

17. Use of a compound as claimed in any one of claims 1 to 11 or a pharmaceutically acceptable salt thereof, in the manufacture of a medicament for treating chronic obstructive pulmonary disease.

18. Use of a compound as claimed in any one of claims 1 to 11 or a pharmaceutically acceptable salt thereof, in the manufacture of a medicament for treating asthma.

19. Use of a compound of formula (I), or a pharmaceutically acceptable salt thereof, as claimed in any one of claims 1 to 11 in the manufacture of a medicament for the treatment of human diseases or conditions in which modulation of CCR1 is beneficial.
